(19) United States
(12) Patent Application Publication
(10) Pub. No.: US 2024/0218399 A1
(43) Pub. Date: Jul. 4, 2024

(54) LIPID COMPOUNDS AND LIPID NANOPARTICLE COMPOSITIONS

(71) Applicant: SUZHOU ABOGEN BIOSCIENCES CO., LTD., Suzhou, Jiangsu (CN)

(72) Inventors: Dandan LING, Suzhou, Jiangsu (CN); Xiaobin PAN, Suzhou, Jiangsu (CN); Jerry ZHANG, Suzhou, Jiangsu (CN); Bo YING, Suzhou, Jiangsu (CN)

(21) Appl. No.: 18/556,299

(22) PCT Filed: Oct. 7, 2022
(86) PCT No.: PCT/CN2022/123721

§ 371 Date: Oct. 19, 2023

(30) Foreign Application Priority Data
Oct. 8, 2021 (WO) PCT/CN2021/122690
Sep. 9, 2022 (WO) PCT/CN2022/117968

(51) Int. Cl. C12N 15/88

(52) U.S. Cl. CPC C12N 15/88

(57) ABSTRACT

Provided herein are sphingomyelin-containing compositions, and related nanoparticle compositions, lipid nanoparticles, including lipid raft nanoparticles. Also provided herein are related methods and uses of the nanoparticle compositions for delivering nucleic acid molecules to achieve enhanced nucleic acid expression.

This application claims the benefit of priority of PCT/CN2021/122690 filed on Oct. 8, 2021, and the PCT Patent Application No. PCT/CN2022/117968 filed on Sep. 9, 2022 the content of which is herein incorporated by reference in its entirety.

### 1. FIELD

The present disclosure generally relates to lipid compounds that can be used in combination with other lipid components, such as neutral lipids, cholesterol and polymer conjugated lipids, to form lipid nanoparticles for delivery of therapeutic agents (e.g., nucleic acid molecules, including nucleic acid mimics such as locked nucleic acids (LNAs), peptide nucleic acids (PNAs), and morpholinos), both in vitro and in vivo, for therapeutic or prophylactic purposes, including vaccination.

### 2. BACKGROUND

Therapeutic nucleic acids have the potential to revolutionize vaccination, gene therapies, protein replacement therapies, and other treatments of genetic diseases. Since the commencement of the first clinical studies on therapeutic nucleic acids in the 2000s, significant progresses have been made through the design of nucleic acid molecules and delivery methods thereof. However, nucleic acid therapeutics still face several challenges, including low cell permeability and high susceptibility to degradation of certain nucleic acids molecules, including RNAs. Thus, there exists a need to develop new methods and compositions that facilitate delivery of nucleic acid molecules in vitro or in vivo for therapeutic and/or prophylactic purposes.

### 3. SUMMARY

Provided herein are lipid compounds, including pharmaceutically acceptable salts, prodrugs or stereoisomers thereof, which can be used alone or in combination with other lipid components such as neutral lipids, charged lipids, steroids (including for example, all sterols) and/or their analogs, and/or polymer conjugated lipids and/or polymers to form lipid nanoparticles for the delivery of therapeutic agents (e.g., nucleic acid molecules, including nucleic acid mimics such as locked nucleic acids (LNAs), peptide nucleic acids (PNAs), and morpholinos). In some instances, the lipid nanoparticles are used to deliver nucleic acids such as antisense and/or messenger RNA.

Provided herein are sphingomyelin-containing compositions and related methods, nanoparticle compositions, and lipid nanoparticles. In some embodiments, the sphingomyelin-containing compositions are formulated as nanoparticle compositions comprising a plurality of lipid nanoparticles. In some embodiments, the lipid nanoparticles comprise lipid raft nanoparticles (LRNP) as described herein.

In one aspect, provided herein are nanoparticle compositions. In some embodiments, the nanoparticle composition comprising a plurality of lipid nanoparticles, wherein the lipid nanoparticles comprise: (a) a sphingomyelin of about 5 to 40 mol percent of the total lipid present in the nanoparticle composition; (b) a cationic lipid; (c) a steroid; (d) a polymer conjugated lipid; and (e) a nucleic acid.

In some embodiments, the sphingomyelin is about 10 to 40 mol percent of the total lipid present in the nanoparticle composition. In some embodiments, the sphingomyelin is about 10 to 30 mol percent of the total lipid present in the nanoparticle composition. In some embodiments, the sphingomyelin is about 10 to 25 mol percent of the total lipid present in the nanoparticle composition. In some embodiments, the sphingomyelin is about 10 to 20 mol percent of the total lipid present in the nanoparticle composition. In some embodiments, the sphingomyelin is about 10 to 15 mol percent of the total lipid present in the nanoparticle composition. In some embodiments, the sphingomyelin is about 10 mol percent of the total lipid present in the nanoparticle composition. In some embodiments, the sphingomyelin is about 15 mol percent of the total lipid present in the nanoparticle composition. In some embodiments, the sphingomyelin is about 20 mol percent of the total lipid present in the nanoparticle composition.

In some embodiments, the cationic lipid is about 30-55 mol percent of the total lipid present in the nanoparticle composition. In some embodiments, the cationic lipid is about 35 to 50 mol percent of the total lipid present in the nanoparticle composition. In some embodiments, the cationic lipid is about 40 to 50 mol percent of the total lipid present in the nanoparticle composition. In some embodiments, the cationic lipid is about 45 to 50 mol percent of the total lipid present in the nanoparticle composition. In some embodiments, the cationic lipid is about 40 mol percent of the total lipid present in the nanoparticle composition. In some embodiments, the cationic lipid is about 45 mol percent of the total lipid present in the nanoparticle composition. In some embodiments, the cationic lipid is about 50 mol percent of the total lipid present in the nanoparticle composition.

In some embodiments, the sphingomyelin of about 10 to 20 mol percent of the total lipid present in the nanoparticle composition, and wherein the cationic lipid is about 40 to 50 mol percent of the total lipid present in the nanoparticle composition. In some embodiments, the sphingomyelin of about 10 to 15 mol percent of the total lipid present in the nanoparticle composition, and wherein the cationic lipid is about 45 mol percent of the total lipid present in the nanoparticle composition. In some embodiments, the sphingomyelin of about 10 to 15 mol percent of the total lipid present in the nanoparticle composition, and wherein the cationic lipid is about 40 mol percent of the total lipid present in the nanoparticle composition. In some embodiments, the sphingomyeiin of about 10 mol percent of the total lipid present in the nanoparticle composition, and wherein the cationic lipid is about 50 mol percent of the total lipid present in the nanoparticle composition. In some embodiments, the sphingomyelin of about 10 mol percent of the total lipid present in the nanoparticle composition, and wherein the cationic lipid is about 45 mol percent of the total lipid present in the nanoparticle composition. In some embodiments, the sphingomyelin of about 15 mol percent of the total lipid present in the nanoparticle composition, and wherein the cationic lipid is about 45 mol percent of the total lipid present in the nanoparticle composition.

In some embodiments, the steroid is about 20 to 50 mol percent of the total lipid present in the nanoparticle composition. In some embodiments, the steroid is about 30 to 50 mol percent of the total lipid present in the nanoparticle composition. In some embodiments, the steroid is about 35 to 45 mol percent of the total lipid present in the nanoparticle composition. In some embodiments, the steroid is about 33.5 to 43.5 mol percent of the total lipid present in the nanoparticle composition. In some embodiments, the steroid is about 33.5 mol percent of the total lipid present in the nanoparticle composition. In some embodiments, the steroid is about 38.5 mol percent of the total lipid present in the nanoparticle composition. In some embodiments, the steroid is about 43.5 mol percent of the total lipid present in the nanoparticle composition.

In some embodiments, the sphingomyelin is about 10-20 mol percent of the total lipid present in the nanoparticle composition, wherein the cationic lipid is about 40-50 mol percent of the total lipid present in the nanoparticle composition, and wherein the steroid is about 30 to 50 mol percent of the total lipid present in the nanoparticle composition. In some embodiments, the sphingomyelin of about 10 to 20 mol percent of the total lipid present in the nanoparticle composition, wherein the cationic lipid is about 45 mol percent of the total lipid present in the nanoparticle composition; and wherein the steroid is about 33.5 to 43.5 mol percent of the total lipid present in the nanoparticle composition. In some embodiments, the sphingomvelin of about 10 to 20 mol percent of the total lipid present in the nanoparticle composition, wherein the cationic lipid is about 40 mol percent of the total lipid present in the nanoparticle composition; and wherein the steroid is about 38.5 to 48.5 mol percent of the total lipid present in the nanoparticle composition. In some embodiments, the sphingomyelin of about 10 mol percent of the total lipid present in the nanoparticle composition, wherein the cationic lipid is about 50 mol percent of the total lipid present in the nanoparticle composition, and wherein the steroid is about 38.5 mol percent of the total lipid present in the nanoparticle composition. In some embodiments, the sphingomyelin of about 10 mol percent of the total lipid present in the nanoparticle composition, wherein the cationic lipid is about 45 mol percent of the total lipid present in the nanoparticle composition; and wherein the steroid is about 43.5 mol percent of the total lipid present in the nanoparticle composition. In some embodiments, the sphingomvelin of about 15 mol percent of the total lipid present in the nanoparticle composition, wherein the cationic lipid is about 45 mol percent of the total lipid present in the nanoparticle composition, and wherein the steroid is about 38.5 mol percent of the total lipid present in the nanoparticle composition. In some embodiments, the sphingomyelin of about 20 mol percent of the total lipid present in the nanoparticle composition, wherein the cationic lipid is about 45 mot percent of the total lipid present in the nanoparticle composition, and wherein the steroid is about 33.5 mol percent of the total lipid present in the nanoparticle composition. In some embodiments, the sphingomyelin of about 10 mol percent of the total lipid present in the nanoparticle composition, wherein the cationic lipid is about 40 mol percent of the total lipid present in the nanoparticle composition, and wherein the steroid is about 48.5 mol percent of the total lipid present in the nanoparticle composition. In some embodiments, the sphingomyelin of about 15 mol percent of the total lipid present in the nanoparticle composition, wherein the cationic lipid is about 40 mol percent of the total lipid present in the nanoparticle composition, and wherein the steroid is about 43.5 mol percent of the total lipid present in the nanoparticle composition. In some embodiments, the sphingomyelin of about 20 mol percent of the total lipid present in the nanoparticle composition, wherein the cationic lipid is about 40 mol percent of the total lipid present in the nanoparticle composition, and wherein the steroid is about 38.5 mol percent of the total lipid present in the nanoparticle composition.

In some embodiments, the polymer conjugated lipid is about 0.5 to 3 mol percent of the total lipid present in the nanoparticle composition. In some embodiments, the polymer conjugated lipid is about 1.5 mol percent of the total lipid present in the nanoparticle composition.

In some embodiments, the sphingomyelin is about 10 to 20 mol percent of the total lipid present in the nanoparticle composition, wherein the cationic lipid is about 40 to 50 mol percent of the total lipid present in the nanoparticle composition, wherein the steroid is about 30 to 50 mol percent of the total lipid present in the nanoparticle composition, and wherein the polymer conjugated lipid is about 0.5 to 3 mol percent of the total lipid present in the nanoparticle composition. In some embodiments, the sphingomyelin is about 10 mol percent of the total lipid present in the nanoparticle composition; wherein the cationic lipid is about 50 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition. In some embodiments, the sphingomyelin is about 10 mol percent of the total lipid present in the nanoparticle composition; wherein the cationic lipid is about 45 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition. In some embodiments, the sphingomyelin is about 10 mol percent of the total lipid present in the nanoparticle composition; wherein the cationic lipid is about 40 mol percent of the total lipid present in the nanoparticle composition; wherein the steroid is about 48.5 mol percent of the total lipid present in the nanoparticle composition; and wherein the polymer conjugated lipid is about 1.5 mol percent of the total lipid present in the nanoparticle composition. In some embodiments, the sphingomyelin is about 15 mol percent of the total lipid present in the nanoparticle composition; wherein the cationic lipid is about 45 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition. In some embodiments, the sphingomyelin is about 15 mol percent of the total lipid present in the nanoparticle composition; wherein the cationic lipid is about 40 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition. In some embodiments, the sphingomyelin is about 20 mol percent of the total lipid present in the nanoparticle composition; wherein the cationic lipid is about 45 mol percent of the total lipid present in the nanoparticle composition; wherein the steroid is about 33.5 mol percent of the total lipid present in the nanoparticle composition; and wherein the polymer conjugated lipid is about 1.5 mol percent of the total lipid present in the nanoparticle composition. In some embodiments, the sphingomyelin is about 20 mol percent of the total lipid present in the nanoparticle composition; wherein the cationic lipid is about 40 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition. In some embodiments, the sphingomyelin is about 5 mol percent of the total lipid present in the nanoparticle composition; wherein the cationic lipid is about 45 mol percent of the total lipid present in the nanoparticle composition; wherein the steroid is about 48.5 mol percent of the total lipid present in the nanoparticle composition; and wherein the polymer conjugated lipid is about 1.5 mol percent of the total lipid present in the nanoparticle composition.

In some embodiments, the sphingomyelin is about 5 mol percent of the total lipid present in the nanoparticle composition; wherein the cationic lipid is about 45 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition; and wherein the nanoparticle composition further comprises a second phospholipid of about 5 mol percent of the total lipid present in the nanoparticle composition; optionally wherein the second phospholipid is 1,2-distearoyl-sn-glycero-3-phosphocholine (DSPC).

In some embodiments, the sphingomyelin is a sphingomyelin compound. In some embodiments, the sphingomyelin is selected from SM-01, SM-02, SM-03, SM-04, SM-05, SM-06 and SM-07 in Table X.

In some embodiments, the steroid is cholesterol or a cholesterol derivative.

In some embodiments, the cationic lipid is a compound according to any one of the formula selected from 01-I, 01-II, 02-I, 02-II, 03-I, 03-II-A, 03-II-B, 03-II-C, 03-II-D, 04-I, 04-III, 04-IV, 05-I, 06-I, and sub-formulas thereof. In some embodiments, the cationic lipid is a compound selected from the compounds listed in any one of Tables 1 to 5.

In some embodiments, the polymer conjugated lipid is DMG-PEG2000 or DMPE-PEG2000.

In some embodiments, the nucleic acid encodes a RNA or protein; and wherein the amount of RNA or protein expressed from the nucleic acid in a mammalian cell or tissue of a mammal is more than the amount of RNA or protein expressed from the nucleic acid formulated in a reference nanoparticle composition that does not comprise sphingomyelin of about 10 to 40 mol percent of the total lipid present in the reference nanoparticle composition.

In some embodiments, the reference nanoparticle composition contains 1,2-distearoyl-sn-glycero-3-phosphocholine (DSPC) instead of sphingomyelin. In some embodiments, the molar percentage of sphingomyelin in the total lipid present in the nanoparticle composition is the same as the molar percentage of DSPC in the total lipid present in the reference nanoparticle composition. In some embodiments, remaining contents are the same between the nanoparticle composition and the reference nanoparticle composition.

In some embodiments, the amount of RNA or protein expressed from the nucleic acid in a mammalian cell or tissue of a mammal is increased at least 10%, 20%, 30%, 40%, 50%, 60%, 70%, 80%, or 90% compared to the amount of RNA or protein expressed from the nucleic acid formulated in the reference nanoparticle composition. In some embodiments, the nucleic acid is mRNA.

In some embodiments, at least about 50% of the lipid nanoparticles in the plurality of lipid nanoparticles has a semi-lamellar morphology. In some embodiments, at least about 55% of the lipid nanoparticles in the plurality of lipid nanoparticles have a semi-lamellar morphology.

In some embodiments, the mean size of the plurality of lipid nanoparticles is from about 40 nm to about 150 nm. In some embodiments, wherein the mean size of the plurality of lipid nanoparticles is from about 50 nm to about 100 nm. In some embodiments, wherein the mean size of the plurality of particles is about 95 nm.

In some embodiments, the encapsulation efficiency of said nucleic acid is at least about 50%. In some embodiments, the encapsulation efficiency of said nucleic acid is at least about 80%. In some embodiments, the encapsulation efficiency of said nucleic acid is at least about 90%.

In some embodiments, the polydispersity index (PDI) of said lipid nanoparticles is from about 0 to about 0.25. In some embodiments, the PDI of said lipid nanoparticles is less than 0.1. In some embodiments, wherein the PDI of said composition is less than 0.05.

In some embodiments of the nanoparticle composition provided herein, the nanoparticle composition is a sphingomyelin-containing composition as described herein.

In a related aspect, provided herein is a method of expressing a nucleic acid in a cell, and the method comprises (a) formulating the nucleic acid in any of the nanoparticle composition described herein, and (b) delivering the nanoparticle composition to the cell under a suitable condition, and wherein the nucleic acid is expressed by the cell.

In some embodiments, the nucleic acid encodes a RNA, peptide or polypeptide. In a related aspect, the nucleic acid is DNA. In a related aspect, the nucleic acid is mRNA. In some embodiments, the cell is a mammalian cell. In some embodiments, the cell is isolated and the delivering is performed by contacting the nanoparticle composition with the cell. In other embodiments, the cell is in its native environment in a subject, and the delivering is performed by administering an effective amount of the nanoparticle composition to subject. In some embodiments, the subject is human. In some embodiments, the subject is a non-human mammal.

In some embodiments, the method provided herein is a method of expressing an mRNA in a mammalian cell or tissue of a mammal comprising (a) formulating the mRNA within a plurality of lipid nanoparticles in a nanoparticle composition comprising a molar ratio of about 5-40% sphingomyelin, about 30 to 55% cationic lipid; about 20 to 50% steroid; and about 0.5 to 3% polymer conjugated lipid; (b) delivering the nanoparticle composition to the mammalian cells or the mammal; and wherein the delivered mRNA is expressed in the mammalian cell or in the mammal.

In some embodiments, the nanoparticle composition used in the method comprises a molar ratio of about 10-40% sphingomyelin, about 35 to 50% cationic lipid; about 30 to 50% steroid; and about 0.5-2 polymer conjugated lipid. In some embodiments, the nanoparticle composition used in the method comprises a molar ratio of about 10-30% sphingomyelin, about 35 to 45% cationic lipid; about 35 to 45% steroid; and about 1.5 polymer conjugated lipid. In some embodiments, the nanoparticle composition used in the method comprises a molar ratio of about 10% sphingomyelin, about 50% cationic lipid; about 38.5% steroid; and about 1.5% polymer conjugated lipid. In some embodiments, the nanoparticle composition used in the method comprises a molar ratio of about 10% sphingomyelin, about 45% cationic lipid; about 43.5% steroid; and about 1.5% polymer conjugated lipid. In some embodiments, the nanoparticle composition used in the method comprises a molar ratio of about 10% sphingomyelin, about 40% cationic lipid; about 48.5% steroid; and about 1.5% polymer conjugated lipid. In some embodiments, the nanoparticle composition used in the method comprises a molar ratio of about 15% sphingomyelin, about 45% cationic lipid; about 38.5% steroid; and about 1.5% polymer conjugated lipid. In some embodiments, the nanoparticle composition used in the method comprises a molar ratio of about 15% sphingomyelin, about 40% cationic lipid; about 43.5% steroid; and about 1.5% polymer conjugated lipid. In some embodiments, the nanoparticle composition used in the method comprises a molar ratio of about 20% sphingomyelin, about 45% cationic lipid; about 33.5% steroid; and about 1.5% polymer conjugated lipid. In some embodiments, the nanoparticle composition used in the method comprises a molar ratio of about 20% sphingomyelin, about 40% cationic lipid; about 38.5% steroid; and about 1.5% polymer conjugated lipid. In some embodiments, the nanoparticle composition used in the method comprises a molar ratio of about 5% sphingomyelin, about 45% cationic lipid; about 48.5% steroid; and about 1.5% polymer conjugated lipid. In some embodiments, the nanoparticle composition used in the method comprises a molar ratio of about 5% sphingomyelin, about 45% cationic lipid; about 43.5% steroid; about 1.5% polymer conjugated lipid, and about 5% of a second phospholipid lipid that is not sphingomyelin; wherein optionally the second phospholipid 1,2-distearoyl-sn-glycero-3-phosphocholine (DSPC).

In some embodiments of the present method, the sphingomyelin is a sphingomyelin compound. In some embodiments of the present method, the sphingomyelin is selected from SM-01, SM-02, SM-03, SM-04, SM-05, SM-06 and SM-07 in Table X. In some embodiments of the present method, the steroid is cholesterol or a cholesterol derivative. In some embodiments of the present method, the cationic lipid is a compound according to any one of the formula selected from 01-I, 01-II, 02-I, 02-II, 03-I, 03-II-A, 03-II-B, 03-II-C, 03-II-D, 04-I, 04-III, 04-IV, 05-I, 06-I and sub-formulas thereof, or wherein the cationic lipid is a compound selected from the compounds listed in any one of Tables 1 to 5. In some embodiments of the present method, the polymer conjugated lipid is DMG-PEG2000 or DMPE-PEG2000.

In some embodiments of the method provided herein, the nanoparticle composition is a sphingomyelin-containing composition as described herein.

In some embodiments of the method provided herein, the nanoparticle composition is a nanoparticle composition as described herein.

In another related aspect, provided herein is a lipid raft nanoparticle (LRNP) comprising (a) a sphingomyelin; and (b) a steroid; and at least one first lipid component that is not sphingomyelin or steroid, wherein the LRNP has a heterogeneous structure comprising at least one liquid-ordered (Lo) domain comprising the sphingomyelin and the steroid, and at least one liquid-disordered (Ld) region comprising the first lipid component.

In some embodiments, the Lo domain comprises a higher sphingomyelin concentration as compared to the Ld region. In some embodiments, the Lo domain comprises a higher steroid concentration as compared to the Ld region.

In some embodiments, under electron microscopy, the Ld region is electron dense. In some embodiments, under electron microscopy, the Lo domain is not electron dense. In some embodiments, under electron microscopy, the Lo domain assumes a uni-lamellar or multi-lamellar structure. In some embodiments, under electron microscopy, the LRNP assumes a semi-lamellar morphology.

In some embodiments, the LRNP is taken up by a cell at a higher level as compared to a reference particle; optionally wherein the LRNP is endocytosed by the cell.

In some embodiments, the LRNP further comprises a nucleic acid. In some embodiments, the nucleic acid encodes a RNA or protein.

In some embodiments, the amount of RNA or protein expressed from the nucleic acid in a mammalian cell or tissue of a mammal is more than the amount of RNA or protein expressed from the nucleic acid formulated in a nucleic acid-lipid reference particle that has the same lipid composition as the LRNP except that sphingomyelin is replaced by a second phospholipid of equal molar percentage. In some embodiments, the second phospholipid is DSPC.

In some embodiments, the LRNP is in a nanoparticle composition described herein. In some embodiments, the LRNP constitutes about 10%, 20%, 30%, 40%, 50%, 60%, 70%, 80%, 90%, or 95% of all lipid nanoparticles in the nanoparticle composition. In some embodiments, the LRNP constitutes at least 50% of lipid nanoparticles in a nanoparticle composition. In some embodiments, the LRNP constitutes at least 55% of lipid nanoparticles in a nanoparticle composition.

In some embodiments, the LRNP described herein has the same content(s), composition(s), molar ratio(s) or percentage(s) as any of sphingomyelin-containing compositions described herein. In some embodiments, the LRNP described herein has the same content(s), composition(s), molar ratio(s) or percentage(s) as any of the nanoparticle compositions described herein.

For example, in some embodiments, the sphingomyelin is of about 5-40 mol percent of the total lipid present in the LRNP. In some embodiments, the steroid is about 20 to 50 mol percent of the total lipid present in the particle. In some embodiments of the LRNP, the first lipid component comprises (c) a cationic lipid; and (d) a polymer conjugated lipid. In some embodiments, the cationic lipid is about 30 to 55 mol percent of the total lipid present in the particle. In some embodiments, the polymer conjugated lipid is about 0.5 to 3 mol percent of the total lipid present in the particle.

In another related aspect, provided herein is a nanoparticle composition comprising (a) a sphingomyelin of about 5-40 mol percent of the total lipid present in the composition; and (b) a steroid; and at least a first lipid component that is not sphingomyelin or steroid; wherein at least about 50% of the lipid nanoparticles in the composition have a semi-lamellar morphology under electron microscopy. In some embodiments, the first lipid component comprises (c) a cationic lipid; and (d) a polymer conjugated lipid. In some embodiments, the steroid is about 20 to 50 mol percent of the total lipid present in the particle. In some embodiments, the cationic lipid is about 30 to 55 mol percent of the total lipid present in the particle. In some embodiments, the polymer conjugated lipid is about 0.5 to 3 mol percent of the total lipid present in the particle. In some embodiments, the nanoparticle composition further comprises (e) a nucleic acid. In some embodiments, the nanoparticle composition is a sphingomyelin-containing composition as described herein.

In another related aspect, provided herein is a nanoparticle composition comprising (a) a sphingomyelin, (b) a cationic lipid, (c) a steroid; (d) a polymer conjugated lipid; and (e) a nucleic acid, wherein the cationic lipid is a compound selected from the compounds in Table Y. In some embodiments, the steroid is cholesterol. In some embodiments, the polymer conjugated lipid is DMG-PEG. In some embodiments, the sphingomyelin is a sphingomyelin compound. In some embodiments, the sphingomyelin compound is selected from the compounds in Table X. In some embodiments, the nucleic acid molecule encodes a RNA, a peptide or a protein. In some embodiments, the nucleic acid molecule is DNA. In some embodiments, the nucleic acid molecule is a m RNA.

### 4. BRIEF DESCRIPTION OF THE FIGURES

FIG. 1A is a schematic illustration of a biological membrane containing phospholipid-rich liquid-crystalline phase domains and sphingolipid-rich liquid-ordered phase domains (rafts) existing in equilibrium.

FIG. 1B illustrates possible interaction via hydrogen bonding between cholesterol and sphingomyelin in a biological membrane.

FIG. 1C illustrates structural similarity and differences between DSCP and sphingomyelin. Particularly, DSCP lacks the amide group of sphingomyelin that forms the hydrogen bond with cholesterol.

FIG. 2A is a schematic illustration contrasting the heterogeneous appearance of a lipid raft nanoparticles (LRNP) that contains microdomains (rafts) formed by sphingomyelin and other lipids (e.g., cholesterol) in the left with the homogenous appearance of previous described LNPs lacking the rafts in the right.

FIG. 2B shows the semi-lamellar morphology of lipid raft nanoparticles (LRNP) under Cryo-electron microscopy (Cryo-EM). Arrow A points to the lamellar structure in a LRNP and Arrow 1B points to the non-lamellar structure of the LRNP. Scale bar 200 nm.

FIG. 2C shows the electron-dense morphology of lipid nanoparticles under Cryo-electron microscopy (Cryo-EM). Scale bar 200 nm.

FIG. 3A shows the green fluorescent protein (GFP) expression levels (relative light units; RLU) in Hela cells treated with the different LNP formulations containing the mRNA encoding GFP. Untreated cells were included as a negative control

FIG. 3B shows the green fluorescent protein (GFP) expression levels (relative light units; RLU) in Hela cells treated with the different LNP formulations containing the mRNA encoding GFP. Untreated cells were included as a negative control.

FIG. 3C shows the green fluorescent protein (GFP) expression levels (relative light units; RLU) in Hela cells treated with the LNP formulations containing different cationic lipids C1 and Lipid 5, respectively. Untreated cells were included as a negative control.

FIG. 4 shows the hEPO expression levels (μg/ml) in female ICR mice injected with the different LNP formulations containing hEPO mRNA.

FIG. 5 shows the microscopic morphologies of three different LNP formulations from Table 5B under Cryo-electron microscopy (Cryo-EM). Scale bar 200 nm.

FIG. 6 shows the EPO expression levels (μg/ml) in female ICR mice injected with different LNPs in Table 6.6 containing different ratios of Sphingomyelin.

FIG. 7 shows the EPO expression levels (μg/ml) in female ICR mice injected with different LNPs in Table 6.7 containing Sphingomyelin of different chain length.

FIG. 8 shows the green fluorescent protein (GFP) expression levels (relative light units; RLU) in Hela cells treated with the LNP formulations in Table 6.8.1 containing either C2 or C3. Untreated cells were included as a negative control.

FIG. 9 shows the EPO expression levels (μg/ml) in female ICR mice injected with LNPs in Table 6.8.2 to study the effect of different cationic lipids.

FIG. 10 show the tissue biodistribution of different LNPs in Table 6.9 containing mRNA encoding luciferase in female ICR mice as measured by ex vivo tissue imaging in terms of total flux (p/s) of each tissue.

### 5. DETAILED DESCRIPTION

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein and unless otherwise specified, the term “lipid” refers to a group of organic compounds that include, but are not limited to, esters of fatty acids and are generally characterized by being poorly soluble in water, but soluble in many nonpolar organic solvents. While lipids generally have poor solubility in water, there are certain categories of lipids (e.g., lipids modified by polar groups, e.g., DMG-PEG2000) that have limited aqueous solubility and can dissolve in water under certain conditions. Known types of lipids include biological molecules such as fatty acids, waxes, sterols, fat-soluble vitamins, monoglycerides, diglycerides, triglycerides, and phospholipids. Lipids can be divided into at least three classes: (1) “simple lipids,” which include fats and oils as well as waxes; (2) “compound lipids,” which include phospholipids and glycolipids (e.g., DMPE-PEG2000); and (3) “derived lipids” such as steroids. Further, as used herein, lipids also encompass lipidoid compounds. The term “lipidoid compound,” also simply “lipidoid”, refers to a lipid-like compound (e.g. an amphiphilic compound with lipid-like physical properties).

The term “lipid nanoparticle” or “LNP” refers to a particle having at least one dimension on the order of nanometers (nm) (e.g., 1 to 1,000 nm), which contains one or more types of lipid molecules. The LNP provided herein can further contain at least one non-lipid payload molecule (e.g., one or more nucleic acid molecules). In some embodiments, the LNP comprises a non-lipid payload molecule either partially or completely encapsulated inside a lipid shell. Particularly, in some embodiments, wherein the payload is a negatively charged molecule (e.g., mRNA encoding a viral protein), and the lipid components of the LNP comprise at least one cationic lipid. Without being bound by the theory, it is contemplated that the cationic lipids can interact with the negatively charged payload molecules and facilitates incorporation and/or encapsulation of the payload into the LNP during LNP formation. Other lipids that can form part of a LNP as provided herein include but are not limited to neutral lipids and charged lipids, such as steroids, polymer conjugated lipids, and various zwitterionic lipids. In certain embodiments, a LNP according to the present disclosure comprises sphingomyelin as described herein.

The term “cationic lipid” refers to a lipid that is either positively charged at any pH value or hydrogen ion activity of its environment, or capable of being positively charged in response to the pH value or hydrogen ion activity of its environment (e.g., the environment of its intended use). Thus, the term “cationic” encompasses both “permanently cationic” and “cationisable.” In certain embodiments, the positive charge in a cationic lipid results from the presence of a quaternary nitrogen atom. In certain embodiments, the cationic lipid comprises a zwitterionic lipid that assumes a positive charge in the environment of its intended use (e.g., at physiological pH). In certain embodiments, the cationic lipid is one or more lipids of Formula 01-I, 01-II, 02-I, 02-II, 03-I, 03-II-A, 03-II-B, 03-II-C, 03-II-D, 04-I, 04-III, 04-IV, 05-I, and 06-I (and sub-formulas thereof) as described herein.

The term “polymer conjugated lipid” refers to a molecule comprising both a lipid portion and a polymer portion. An example of a polymer conjugated lipid is a pegylated lipid (PEG-lipid), in which the polymer portion comprises a polyethylene glycol.

The term “neutral lipid” encompasses any lipid molecules existing in uncharged forms or neutral zwitterionic forms at a selected pH value or within a selected pH range. In some embodiments, the selected useful pH value or range corresponds to the pH condition in an environment of the intended uses of the lipids, such as the physiological pH. As non-limiting examples, neutral lipids that can be used in connection with the present disclosure include, but are not limited to, phosphotidylcholines such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), 2-((2,3-bis(oleoyloxy)propyl)dimethylammonio)ethyl hydrogen phosphate (DOCP), sphingomyelins (SM), ceramides, steroids such as sterols and their derivatives. Neutral lipids as provided herein may be synthetic or derived (isolated or modified) from a natural source or compound.

The term “charged lipid” encompasses any lipid molecules that exist in either positively charged or negatively charged forms at a selected pH or within a selected pH range. In some embodiments, the selected pH value or range corresponds to the pH condition in an environment of the intended uses of the lipids, such as the physiological pH. As non-limiting examples, neutral lipids that can be used in connection with the present disclosure include, but are not limited to, phosphatidylserines, phosphatidic acids, phosphatidylglycerols, phosphatidylinositols, sterol hemisuccinates, dialkyl trimethylammonium-propanes, (e.g., DOTAP, DOTMA), dialkyl dimethylaminopropanes, ethyl phosphocholines, dimethylaminoethane carbamoyl sterols (e.g., DC-Chol), 1,2-dioleoyl-sn-glycero-3-phospho-L-serine sodium salt (DOPS-Na), 1,2-dioleoyl-sn-glycero-3-phospho-(1′-rac-glycerol) sodium salt (DOPG-Na), and 1,2-dioleoyl-sn-glycero-3-phosphate sodium salt (DOPA-Na). Charged lipids as provided herein may be synthetic or derived (isolated or modified) from a natural source or compound.

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to twenty-four carbon atoms (C₁-C₂₄ alkyl), four to twenty carbon atoms (C₁-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, and the like. Unless otherwise specified, an alkenyl group is optionally substituted.

As used herein, and unless otherwise specified, the term “alkynyl” refers to a straight or branched hydrocarbon chain radical consisting solely of carbon and hydrogen atoms, which contains one or more carbon-carbon triple bonds. In one embodiment, the alkynyl group has, for example, from two to twenty-four carbon atoms (C₂-C₂₄ alkynyl), four to twenty carbon atoms (C₄-C₂₀ alkynyl), six to sixteen carbon atoms (C₆-C₁₆ alkynyl), six to nine carbon atoms (C₆-C₉ alkynyl), two to fifteen carbon atoms (C₂-C₁₅ alkynyl), two to twelve carbon atoms (C₂-C₁₂alkynyl), two to eight carbon atoms (C₂-C₈ alkynyl) or two to six carbon atoms (C₂-C₆ alkynyl) and which is attached to the rest of the molecule by a single bond. Examples of alkynyl groups include, but are not limited to, ethynyl, propynyl, butynyl, pentynyl, and the like. Unless otherwise specified, an alkynyl group is optionally substituted.

As used herein, and unless otherwise specified, the term “alkylene” or “alkylene chain” refers to a straight or branched multivalent (e.g., divalent or trivalent) hydrocarbon chain linking the rest of the molecule to a radical group, consisting solely of carbon and hydrogen, which is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene). Examples of alkylene groups include, but are not limited to, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless otherwise specified, an alkylene chain is optionally substituted.

As used herein, and unless otherwise specified, the term “alkenylene” refers to a straight or branched multivalent (e.g., divalent or trivalent) hydrocarbon chain linking the rest of the molecule to a radical group, consisting solely of carbon and hydrogen, which contains one or more carbon-carbon double bonds. In one embodiment, the alkenylene has, for example, from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₈ alkenylene), two to six carbon atoms (C₂-C₈ alkenylene) or two to four carbon atoms (C₂-C₄ alkenylene). Examples of alkenylene include, but are not limited to, ethenylene, propenylene, n-butenylene, and the like. The alkenylene is attached to the rest of the molecule through a single or double bond and to the radical group through a single or double bond. The points of attachment of the alkenylene to the rest of the molecule and to the radical group can be through one carbon or any two carbons within the chain. Unless otherwise specified, an alkenylene is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused or bridged ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooetyl. Examples of polycyclic cycloalkyl radicals include, but are not limited to, adamantyl, norbornyl, decalinyl, 7,7-dimethyl-bicyclo[2.2.1]heptanyl, and the like. Unless otherwise specified, a cycloalkyl group is optionally substituted.

As used herein, and unless otherwise specified, the term “cycloalkylene” is a multivalent (e.g., divalent or trivalent) cycloalkyl group. Unless otherwise specified, a cycloalkylene group is optionally substituted.

As used herein, and unless otherwise specified, the term “cycloalkenyl” refers to a non-aromatic monocyclic or polycyclic hydrocarbon radical consisting solely of carbon and hydrogen atoms, and which includes one or more carbon-carbon double bonds. Cycloalkenyl may include fused or bridged ring systems. In one embodiment, the cycloalkenyl has, for example, from 3 to 15 ring carbon atoms (C₃-C₁₅ cycloalkenyl), from 3 to 10 ring carbon atoms (C₃-C₁₀ cycloalkenyl), or from 3 to 8 ring carbon atoms (C₃-C₈ cycloalkenyl). The cycloalkenyl is attached to the rest of the molecule by a single bond. Examples of monocyclic cycloalkenyl radicals include, but are not limited to, cyclopropenyl, cyclobutenyl, cyclopentenyl, cyclohexenyl, cycloheptenyl, cyclooctenyl, and the like. Unless otherwise specified, a cycloalkenyl group is optionally substituted.

As used herein, and unless otherwise specified, the term “cycloalkenylene” is a multivalent (e.g., divalent or trivalent) cycloalkenyl group. Unless otherwise specified, a cycloalkenylene group is optionally substituted.

As used herein, and unless otherwise specified, the term “heterocyclyl” refers to a non-aromatic radical monocyclic or polycyclic moiety that contains one or more (e.g., one, one or two, one to three, or one to four) heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other polycyclic ring system, wherein the polycyclic ring systems can be a fused, bridged or spiro ring system. Heterocyclyl polycyclic ring systems can include one or more heteroatoms in one or more rings. A heterocyclyl group can be saturated or partially unsaturated. Saturated heterocycloalkyl groups can be termed “heterocycloalkyl”. Partially unsaturated heterocycloalkyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 18 ring atoms (3- to 18-membered heterocyclyl), 4 to 8 ring atoms (4- to 8-membered heterocyclyl), or 5 to 8 ring atoms (5- to 8-membered heterocyclyl). Whenever it appears herein, a numerical range such as “3 to 18” refers to each integer in the given range; e.g., “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Examples of heterocyclyl groups include, but are not limited to, imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl. Unless otherwise specified, a heterocyclyl group is optionally substituted.

As used herein, and unless otherwise specified, the term “heterocyclylene” is a multivalent (e.g., divalent or trivalent) heterocyclyl group. Unless otherwise specified, a heterocyclylene group is optionally substituted.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

As used herein, and unless otherwise specified, the term “arylene” is a multivalent (e.g., divalent or trivalent) aryl group. Unless otherwise specified, an arylene group is optionally substituted.

As used herein, and unless otherwise specified, the term “heteroaryl” refers to a monocyclic aromatic group and/or multicyclic aromatic group that contains at least one aromatic ring, wherein at least one aromatic ring contains one or more (e.g., one, one or two, one to three, or one to four) heteroatoms independently selected from O, S, and N. The heteroaryl may be attached to the main structure at any heteroatom or carbon atom. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. Unless otherwise specified, a heteroaryl group is optionally substituted.

As used herein, and unless otherwise specified, the term “heteroarylene” is a multivalent (e.g., divalent or trivalent) heteroaryl group. Unless otherwise specified, a heteroarylene group is optionally substituted.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, CI, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(O)R′; —NR′R′; —NR′C(═O)R′; —C(═O)NR′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

As used herein, and unless otherwise specified, the term “prodrug” of a biologically active compound refers to a compound that may be converted under physiological conditions or by solvolysis to the biologically active compound. In one embodiment, the term “prodrug” refers to a metabolic precursor of the biologically active compound that is pharmaceutically acceptable. A prodrug may be inactive when administered to a subject in need thereof, but is converted in vivo to the biologically active compound. Prodrugs are typically rapidly transformed in vivo to yield the parent biologically active compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

In one embodiment, the term “prodrug” is also meant to include any covalently bonded carriers, which release the active compound in vivo when such prodrug is administered to a mammalian subject. Prodrugs of a compound may be prepared by modifying functional groups present in the compound in such a way that the modifications are cleaved, either in routine manipulation or in vivo, to the parent compound. Prodrugs include compounds wherein a hydroxyl, amino or mercapto group is bonded to any group that, when the prodrug of the compound is administered to a mammalian subject, cleaves to form a free hydroxyl, free amino or free mercapto group, respectively.

Examples of prodrugs include, but are not limited to, acetate, formate and benzoate derivatives of alcohol or amide derivatives of amine functional groups in the compounds provided herein.

As used herein, and unless otherwise specified, the term “pharmaceutically acceptable salt” includes both acid and base addition salts.

Examples of pharmaceutically acceptable acid addition salts include, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, glu conic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Examples of pharmaceutically acceptable base addition salt include, but are not limited to, salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

A compound provided herein may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Unless otherwise specified, a compound provided herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two assymetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

It should also be noted a compound described herein can contain unnatural proportions of atomic isotopes at one or more of the atoms. For example, the compounds may be radiolabeled with radioactive isotopes, such as for example tritium (³H), iodine-125 (¹²⁵I) sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound described herein, whether radioactive or not, are intended to be encompassed within the scope of the embodiments provided herein. In some embodiments, there are provided isotopologs of a compound described herein, for example, the isotopologs are deuterium, carbon-13, and/or nitrogen-15 enriched. As used herein, “deuterated”, means a compound wherein at least one hydrogen (H) has been replaced by deuterium (indicated by D or ²H), that is, the compound is enriched in deuterium in at least one position.

It should be noted that if there is a discrepancy between a depicted structure and a name for that structure, the depicted structure is to be accorded more weight.

As used herein, and unless otherwise specified, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “composition” is intended to encompass a product containing the specified ingredients (e.g., a mRNA molecule provided herein) in, optionally, the specified amounts.

As used herein, “mol percent” refers to a component's molar percentage relative to total mols of all lipid components in the nanoparticle composition (e.g., total mols of the sphingomyelin, and the steroid, and cationic lipid(s), and the neutral lipid, and the polymer conjugated lipid, etc.).

The term “polynucleotide” or “nucleic acid,” as used interchangeably herein, refers to polymers of nucleotides of any length and includes, e.g., DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. Nucleic acid can be in either single- or double-stranded forms. As used herein and unless otherwise specified, “nucleic acid” also includes nucleic acid mimics such as locked nucleic acids (LNAs), peptide nucleic acids (PNAs), and morpholinos. “Oligonucleotide,” as used herein, refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “non-naturally occurring” when used in reference to a nucleic acid molecule as described herein is intended to mean that the nucleic acid molecule is not found in nature. A non-naturally occurring nucleic acid encoding a viral peptide or protein contains at least one genetic alternation or chemical modification not normally found in a naturally occurring strain of the virus, including wild-type strains of the virus. Genetic alterations include, for example, modifications introducing expressible nucleic acid sequences encoding peptides or polypeptides heterologous to the virus, other nucleic acid additions, nucleic acid deletions, nucleic acid substitution, and/or other functional disruption of the virus' genetic material. Such modifications include, for example, modifications in the coding regions and functional fragments thereof, for heterologous, homologous or both heterologous and homologous polypeptides for the viral species. Additional modifications include, for example, modifications in non-coding regulatory regions in which the modifications alter expression of a gene or operon. Additional modifications also include, for example, incorporation of a nucleic acid sequence into a vector, such as a plasmid or an artificial chromosome. Chemical modifications include, for example, one or more functional nucleotide analog as described herein.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as an mRNA molecule, can be substantially free of other cellular material, or culture medium when produced by recombinant techniques, or substantially free of chemical precursors or other chemicals when chemically synthesized. In a specific embodiment, one or more nucleic acid molecules encoding an antigen as described herein are isolated or purified. The term embraces nucleic acid sequences that have been removed from their naturally occurring environment, and includes recombinant or cloned DNA or RNA isolates and chemically synthesized analogues or analogues biologically synthesized by heterologous systems. A substantially pure molecule may include isolated forms of the molecule.

The term “encoding nucleic acid” or grammatical equivalents thereof as it is used in reference to nucleic acid molecule encompasses (a) a nucleic acid molecule in its native state or when manipulated by methods well known to those skilled in the art that can be transcribed to produce mRNA which is then translated into a peptide and/or polypeptide, and (b) the mRNA molecule itself. The antisense strand is the complement of such a nucleic acid molecule, and the encoding sequence can be deduced therefrom. The term “coding region” refers to a portion in an encoding nucleic acid sequence that is translated into a peptide or polypeptide. The term “untranslated region” or “UTR” refers to the portion of an encoding nucleic acid that is not translated into a peptide or polypeptide. Depending on the orientation of a UTR with respect to the coding region of a nucleic acid molecule, a UTR is referred to as the 5′-UTR if located to the 5′-end of a coding region, and a UTR is referred to as the 3′-UTR if located to the 3′-end of a coding region.

The term “mRNA” as used herein refers to a message RNA molecule comprising one or more open reading frame (ORF) that can be translated by a cell or an organism provided with the mRNA to produce one or more peptide or protein product. The region containing the one or more ORFs is referred to as the coding region of the mRNA molecule. In certain embodiments, the mRNA molecule further comprises one or more untranslated regions (UTRs).

In certain embodiments, the mRNA is a monocistronic mRNA that comprises only one ORF. In certain embodiments, the monocistronic mRNA encodes a peptide or protein comprising at least one epitope of a selected antigen (e.g., a pathogenic antigen or a tumor associated antigen). In other embodiments, the mRNA is a multicistronic mRNA that comprises two or more ORFs. In certain embodiments, the multicistronic mRNA encodes two or more peptides or proteins that can be the same or different from each other. In certain embodiments, each peptide or protein encoded by a multicistronic mRNA comprises at least one epitope of a selected antigen. In certain embodiments, different peptide or protein encoded by a multicistronic mRNA each comprises at least one epitope of different antigens. In any of the embodiments described herein, the at least one epitope can be at least 2, at least 3, at least 4, at least 5, at least 6, at least 7, at least 8, at least 9, or at least 10 epitopes of an antigen.

The term “nucleobases” encompasses purines and pyrimidines, including natural compounds adenine, thymine, guanine, cytosine, uracil, inosine, and natural or synthetic analogs or derivatives thereof.

The term “functional nucleotide analog” as used herein refers to a modified version of a canonical nucleotide A, G, C, U or T that (a) retains the base-pairing properties of the corresponding canonical nucleotide, and (b) contains at least one chemical modification to (i) the nucleobase, (ii) the sugar group, (iii) the phosphate group, or (iv) any combinations of (i) to (iii), of the corresponding natural nucleotide. As used herein, base pairing encompasses not only the canonical Watson-Crick adenine-thymine, adenine-uracil, or guanine-cytosine base pairs, but also base pairs formed between canonical nucleotides and functional nucleotide analogs or between a pair of functional nucleotide analogs, wherein the arrangement of hydrogen bond donors and hydrogen bond acceptors permits hydrogen bonding between a modified nucleobase and a canonical nucleobase or between two complementary modified nucleobase structures. For example, a functional analog of guanosine (G) retains the ability to base-pair with cytosine (C) or a functional analog of cytosine. One example of such non-canonical base pairing is the base pairing between the modified nucleotide inosine and adenine, cytosine, or uracil. As described herein, a functional nucleotide analog can be either naturally occurring or non-naturally occurring. Accordingly, a nucleic acid molecule containing a functional nucleotide analog can have at least one modified nucleobase, sugar group and/or internucleoside linkage. Exemplary chemical modifications to the nucleobases, sugar groups, or internucleoside linkages of a nucleic acid molecule are provided herein.

The terms “translational enhancer element,” “TEE” and “translational enhancers” as used herein refers to an region in a nucleic acid molecule that functions to promotes translation of a coding sequence of the nucleic acid into a protein or peptide product, such as via cap-dependent or cap-independent translation. A TEE typically locates in the UTR region of a nucleic acid molecule (e.g., mRNA) and enhance the translational level of a coding sequence located either upstream or downstream. For example, a TEE in a 5′-UTR of a nucleic acid molecule can locate between the promoter and the starting codon of the nucleic acid molecule. Various TEE sequences are known in the art (Wellensiek et at. Genome-wide profiling of human cap-independent translation-enhancing elements, Nature Methods, 2013 August; 10(8): 747-750; Chappell et al. PNAS Jun. 29, 2004 101 (26) 9590-9594). Some TEEs are known to be conserved across multiple species (Pánek et al. Nucleic Acids Research, Volume 41, Issue 16, 1 September 2013, Pages 7625-7634).

As used herein, the term “stem-loop sequence” refers to a single-stranded polynucleotide sequence having at least two regions that are complementary or substantially complementary to each other when read in opposite directions, and thus capable of base-pairing with each other to form at least one double helix and an unpaired loop. The resulting structure is known as a stem-loop structure, a hairpin, or a hairpin loop, which is a secondary structure found in many RNA molecules.

The term “peptide” as used herein refers to a polymer containing between two and fifty (2-50) amino acid residues linked by one or more covalent peptide bond(s). The terms apply to naturally occurring amino acid polymers as well as amino acid polymers in which one or more amino acid residues is a non-naturally occurring amino acid (e.g., an amino acid analog or non-natural amino acid).

The terms “polypeptide” and “protein” are used interchangeably herein to refer to a polymer of greater than fifty (50) amino acid residues linked by covalent peptide bonds. That is, a description directed to a polypeptide applies equally to a description of a protein, and vice versa. The terms apply to naturally occurring amino acid polymers as well as amino acid polymers in which one or more amino acid residues is a non-naturally occurring amino acid (e.g., an amino acid analog). As used herein, the terms encompass amino acid chains of any length, including full length proteins (e.g., antigens).

In the context of a peptide or polypeptide, the term “derivative” as used herein refers to a peptide or polypeptide that comprises an amino acid sequence of the viral peptide or protein, or a fragment of a viral peptide or protein, which has been altered by the introduction of amino acid residue substitutions, deletions, or additions. The term “derivative” as used herein also refers to a viral peptide or protein, or a fragment of a viral peptide or protein, which has been chemically modified, e.g., by the covalent attachment of any type of molecule to the polypeptide. For example, but not by way of limitation, a viral peptide or protein or a fragment of the viral peptide or protein may be chemically modified, e.g., by glycosylation, acetylation, pegylation, phosphorylation, amidation, derivatization by known protecting/blocking groups, proteolytic cleavage, chemical cleavage, formulation, metabolic synthesis of tunicamycin, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the viral peptide or protein. Further, a derivative of a viral peptide or protein or a fragment of a viral peptide or protein may contain one or more non-classical amino acids. In specific embodiments, a derivative is a functional derivative of the native or unmodified peptide or polypeptide from which it was derived.

The term “functional derivative” refers to a derivative that retains one or more functions or activities of the naturally occurring or starting peptide or polypeptide from which it was derived. For example, a functional derivative of a coronavirus S protein may retain the ability to bind one or more of its receptors on a host cell. For example, a functional derivative of a coronavirus N protein may retain the ability to bind RNA or the package viral genome.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, fragments refers to polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a polypeptide. In a specific embodiment, a fragment of a polypeptide retains at least 1, at least 2, at least 3, or more functions of the polypeptide.

The term “genetic vaccine” as used herein refers to a therapeutic or prophylactic composition comprising at least one nucleic acid molecule encoding an antigen associated with a target disease (e.g., an infectious disease or a neoplastic disease). Administration of the vaccine to a subject (“vaccination”) allows for the production of the encoded peptide or protein, thereby eliciting an immune response against the target disease in the subject. In certain embodiments, the immune response comprises adaptive immune response, such as the production of antibodies against the encoded antigen, and/or activation and proliferations of immune cells capable of specifically eliminating diseased cells expressing the antigen. In certain embodiments, the immune response further comprises innate immune response. According to the present disclosure, a vaccine can be administered to a subject either before or after the onset of clinical symptoms of the target disease. In some embodiments, vaccination of a healthy or asymptomatic subject renders the vaccinated subject immune or less susceptible to the development of the target disease. In some embodiments, vaccination of a subject showing symptoms of the disease improves the condition of, or treats, the disease in the vaccinated subject.

The terms “innate immune response” and “innate immunity” are recognized in the art, and refer to non-specific defense mechanism a body's immune system initiates upon recognition of pathogen-associated molecular patterns, which involves different forms of cellular activities, including cytokine production and cell death through various pathways. As used herein, innate immune responses include, without limitation, increased production of inflammation cytokines (e.g., type I interferon or IL-10 production), activation of the NFκB pathway, increased proliferation, maturation, differentiation and/or survival of immune cells, and in some cases, induction of cell apoptosis. Activation of the innate immunity can be detected using methods known in the art, such as measuring the (NF)-κB activation.

The terms “adaptive immune response” and “adaptive immunity” are recognized in the art, and refer to antigen-specific defense mechanism a body's immune system initiates upon recognition of a specific antigen, which include both humoral response and cell-mediated responses. As used herein, adaptive immune responses include cellular responses that is triggered and/or augmented by a vaccine composition, such as a genetic composition described herein. In some embodiments, the vaccine composition comprises an antigen that is the target of the antigen-specific adaptive immune response. In other embodiments, the vaccine composition, upon administration, allows the production in an immunized subject of an antigen that is the target of the antigen-specific adaptive immune response. Activation of an adaptive immune response can be detected using methods known in the art, such as measuring the antigen-specific antibody production, or the level of antigen-specific cell-mediated cytotoxicity.

The term “administer” or “administration” refers to the act of injecting or otherwise physically delivering a substance as it exists outside the body (e.g., a lipid nanoparticle composition as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

“Chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

The term “targeted delivery” or the verb form “target” as used herein refers to the process that promotes the arrival of a delivered agent (such as a therapeutic payload molecule in a lipid nanoparticle composition as described herein) at a specific organ, tissue, cell and/or intracellular compartment (referred to as the targeted location) more than any other organ, tissue, cell or intracellular compartment (referred to as the non-target location). Targeted delivery can be detected using methods known in the art, for example, by comparing the concentration of the delivered agent in a targeted cell population with the concentration of the delivered agent at a non-target cell population after systemic administration. In certain embodiments, targeted delivery results in at least 2 fold higher concentration at a targeted location as compared to a non-target location.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including, for example, infection and neoplasia. In some embodiments, the effective amount is a therapeutically effective amount or a prophylactically effective amount.

The term “therapeutically effective amount” as used herein refers to the amount of an agent (e.g., a vaccine composition) that is sufficient to reduce and/or ameliorate the severity and/or duration of a given disease, disorder, or condition, and/or a symptom related thereto (e.g., an infectious disease such as caused by viral infection, or a neoplastic disease such as cancer). A “therapeutically effective amount” of a substance/molecule/agent of the present disclosure (e.g., the lipid nanoparticle composition as described herein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a lipid nanoparticle composition as described herein or a therapeutic or prophylactic agent contained therein (e.g., a therapeutic mRNA) effective to “treat” a disease, disorder, or condition, in a subject or mammal.

A “prophylactically effective amount” is an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s) (e.g., an infectious disease such as caused by viral infection, or a neoplastic disease such as cancer). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

The terms “treat,” “treating,” and “treatment” refer to an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

The terms “prevent,” “preventing,” and “prevention” refer to reducing the likelihood of the onset (or recurrence) of a disease, disorder, condition, or associated symptom(s) (e.g., an infectious disease such as caused by viral infection, or a neoplastic disease such as cancer).

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as a lipid nanoparticle composition as described herein) to “manage” an infectious or neoplastic disease, one or more symptoms thereof, so as to prevent the progression or worsening of the disease.

The term “prophylactic agent” refers to any agent that can totally or partially inhibit the development, recurrence, onset, or spread of disease and/or symptom related thereto in a subject.

The term “therapeutic agent” refers to any agent that can be used in treating, preventing, or alleviating a disease, disorder, or condition, including in the treatment, prevention, or alleviation of one or more symptoms of a disease, disorder, or condition and/or a symptom related thereto.

The term “therapy” refers to any protocol, method, and/or agent that can be used in the prevention, management, treatment, and/or amelioration of a disease, disorder, or condition. In certain embodiments, the terms “therapies” and “therapy” refer to a biological therapy, supportive therapy, and/or other therapies useful in the prevention, management, treatment, and/or amelioration of a disease, disorder, or condition, known to one of skill in the art such as medical personnel.

As used herein, a “prophylactically effective serum titer” is the serum titer of an antibody in a subject (e.g., a human), that totally or partially inhibits the development, recurrence, onset, or spread of a disease, disorder, or condition, and/or symptom related thereto in the subject.

In certain embodiments, a “therapeutically effective serum titer” is the serum titer of an antibody in a subject (e.g., a human), that reduces the severity, the duration, and/or the symptoms associated with a disease, disorder, or condition, in the subject.

The term “serum titer” refers to an average serum titer in a subject from multiple samples (e.g., at multiple time points) or in a population of at least 10, at least 20, at least 40 subjects, up to about 100, 1000, or more.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal (e.g., a human) having an infectious disease or neoplastic disease. In another embodiment, the subject is a mammal (e.g., a human) at risk of developing an infectious disease or neoplastic disease.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as an antigen encoded by an mRNA molecule as described herein, in a sample or subject. A detectable agent can be a substance that is capable of being visualized or a substance that is otherwise able to be determined and/or measured (e.g., by quantitation).

“Substantially all” refers to at least about 60%, at least about 65%, at least about 70%, at least about 75%, at least about 80%, at least about 85%, at least about 90%, at least about 95%, at least about 98%, at least about 99%, or about 100%.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, 0.05%, or less of a given value or range. As used herein, when “about” is used in connection with a numerical range, the term “about” is meant to apply to both ends of such modified range (e.g., “about 5 to 10” means “about 5 to about 10”).

The singular terms “a,” “an,” and “the” as used herein include the plural reference unless the context clearly indicates otherwise.

All publications, patent applications, accession numbers, and other references cited in this specification are herein incorporated by reference in their entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section and examples are intended to illustrate but not limit the scope of invention described in the claims.

### 5.3 Sphingomyelin-Containing Compositions

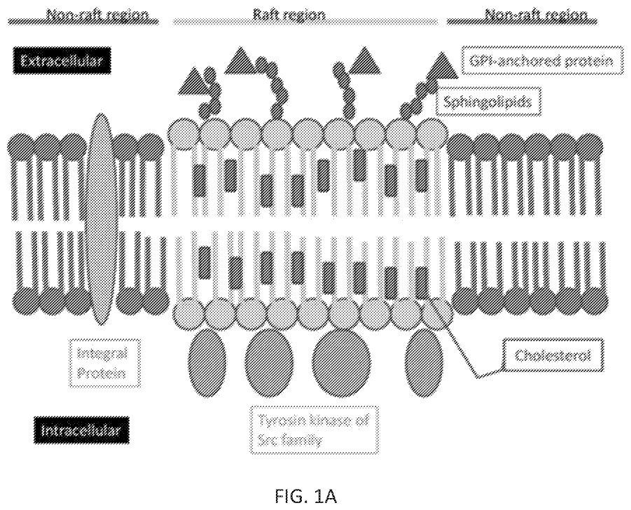

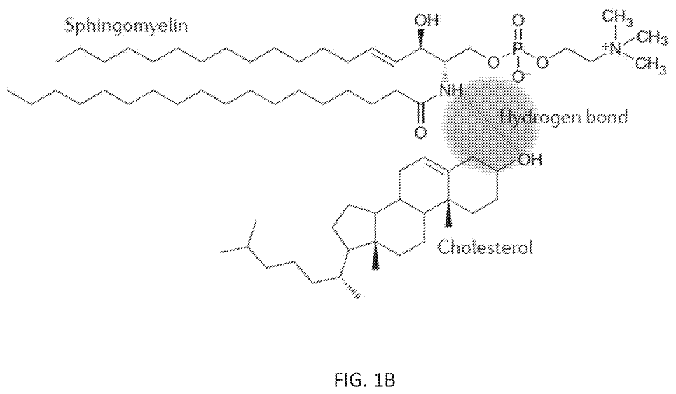

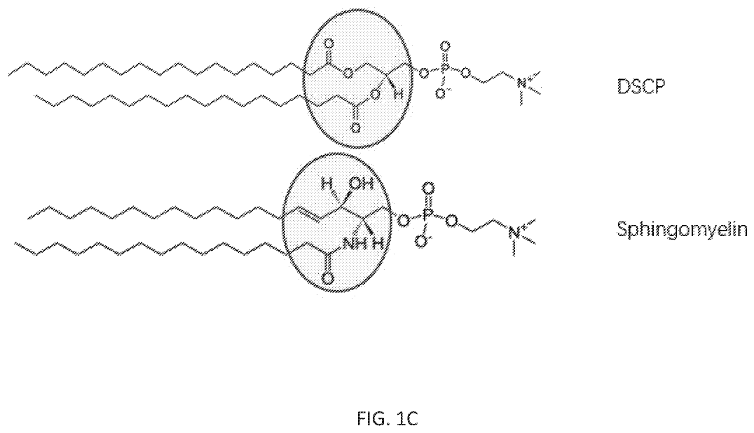

Sphingolipids refer to a class of complex lipids that are composed of a sphingoid long-chain base, a fatty acid tethered to the amino group of the backbone of sphingosine (1,3-dihydroxy-2-amino-4-octadecene), and a variable polar head-group. The sphingosine base and the fatty acid alone constitute ceramide and the linked head-groups can range from phosphocholine (sphingomyelin), to sugars (glycosphingolipids), to complex carbohydrates. As illustrated in FIG. 1A, sphingolipids function as structural components of cell membranes, and tend to associate with each other as well as with cholesterol and certain categories of proteins, such as those attached to the membrane via a glycosylphosphatidylinositol anchor. The aggregates are genetically referred to as “rafts.” Rafts are envisioned to be relatively small, distinct, and liquid-ordered (Lo) subdomains of the plasma membrane that form and disperse rapidly due to the highly saturated alkyl chains and intermolecular hydrogen bonding of sphingolipids.

Mammalian cells usually contains sphingomyelin (e.g., N-stearoyl-D-erythro-sphingosylphosphorylcholine). Natural sphingomyelins usually contain a mixed population with the amide-linked acyl chain differ widely in length (e.g., from 14 to 24 carbons, from 14 to 20 carbons, from 16 to 24 carbons, from 16 to 20 carbons, or 18 carbons). FIG. 1B shows an exemplary sphingomyelin molecule forming a hydrogen bond with cholesterol. As shown in FIG. 1C, the hydrogen bond forming amide of sphingomyelin comes from the sphingosine backbone, which is missing from other structurally similar phospholipids, such as 1,2-distearoyl-sn-glycero-3-phosphocholine (DSPC.

The present disclosure is based, in part, upon the surprising discovery that by formulating lipid nanoparticles (LNPs) using sphingomyelin as a structural lipid, a LNP having a unique semi-lamellar morphology is formed. Without being bound by any theory, it is contemplated that sphingomyelin-rich liquid-ordered domains (rafts) can form and disperse in liquid disordered (Ld) non-raft regions in a lipid nanoparticle, and the heterogeneous nature of these raft-containing particles gives rise to the semi-lamellar morphology under electron microscope. These raft-containing particles are sometimes referred to as lipid raft nanoparticle (LRNP) in the present disclosure.

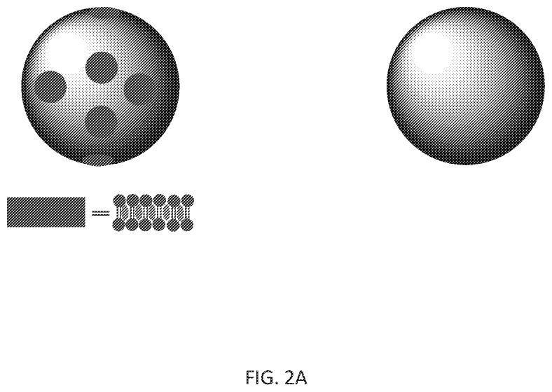

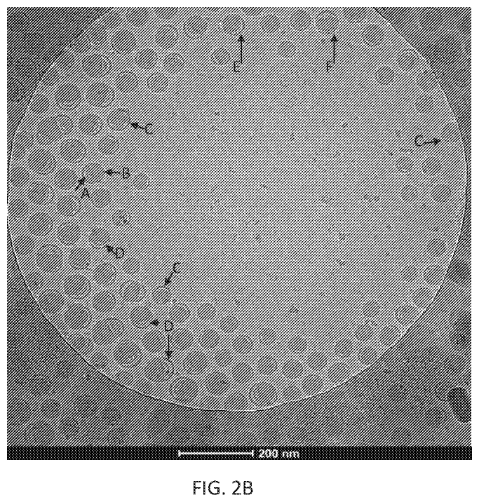

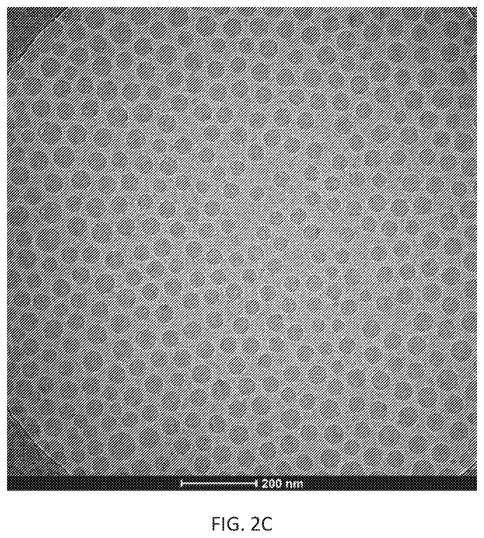

Particularly, FIG. 2A is a schematic illustration contrasting the heterogeneous appearance of a LRNP according to the present disclosure that contains microdomains (rafts) formed by sphingomyelin and other lipids (e.g., cholesterol) in the left with the homogenous appearance of previous described LNPs lacking the rafts in the right.

As used herein, the term “semi-lamellar morphology” of a particle means that the microscopic structure of the particle contains a lamellar (unilamellar or multilamellar) portion (see FIG. 2B area pointed by Arrow A) and an electron dense portion (see FIG. 2B area pointed by Arrow B), where the lamellar portion surrounds less than the entire electron dense core. In some embodiments, a particle having the semi-lamellar morphology can comprise two or more lamellar portions that collectively surround less than the entire electron dense core.

Without being bound by any theory, it is contemplated that that the lamellar portion of a particle has a lipid bilayer structure, and the electron dense portion of a particle has a non-lamellar lipid bilayer structure that can include, for example, three dimensional tubes, rods, cubic symmetries, etc. The semi-lamellar morphology of the resulting lipid particles can readily be determined using techniques known to and used by those of skill in the art. Such techniques include, but are not limited to, Cryo-Transmission Electron Microscopy (“Cryo-TEM”), electron cryomicroscopy (“Cryo-EM”), Differential Scanning calorimetry (“DSC”), X-Ray Diffraction, etc. The term “electron dense” as used herein to describe morphology of a particle means that the particle or a portion of the particle has a density that prevents electrons from penetrating, resulting in a solid dark appearance under electron microscope.

FIGS. 2B and 2C are Cryo-EM images showing the morphology of the present LRNP and a reference LNP that were assessed and characterized using the Cryo-EM analysis described in Example 3 herein. In contrast to the semi-lamellar morphology of the present LRNP, FIG. 2C shows the electron-dense morphology of the reference lipid nanoparticle that has the solid dark appearance in the whole particles.

As shown in FIG. 2B, a semi-lamellar nanoparticle comprises an electron-dense core and at least one lamellar portion, where the lamellar portion surrounds only part of the electron-dense core. Some lamellar portion of a semi-lamellar nanoparticle assumes a single-layer membrane appearance (see FIG. 2B Arrows C), which is referred herein as an “uni-lamellar” structure, while others assume a multiple-layer membrane appearance (see FIG. 2B Arrows D), which is referred herein as “multi-lamellar” structure. A semi-lamellar particle can have one (see FIG. 2B Arrow E) or multiple lamellar portions (see FIG. 2B Arrow F).

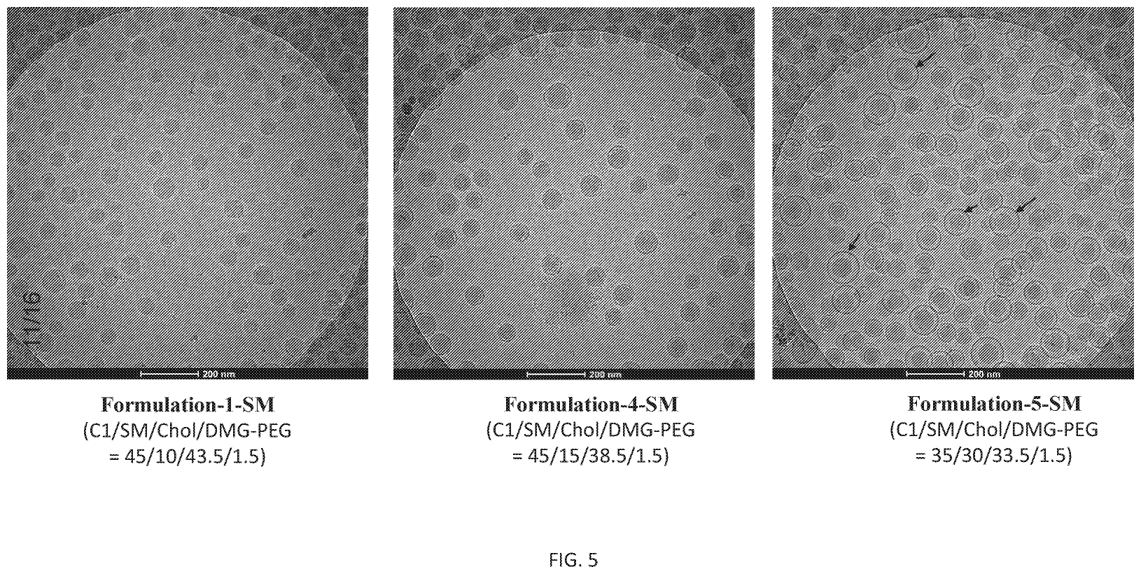

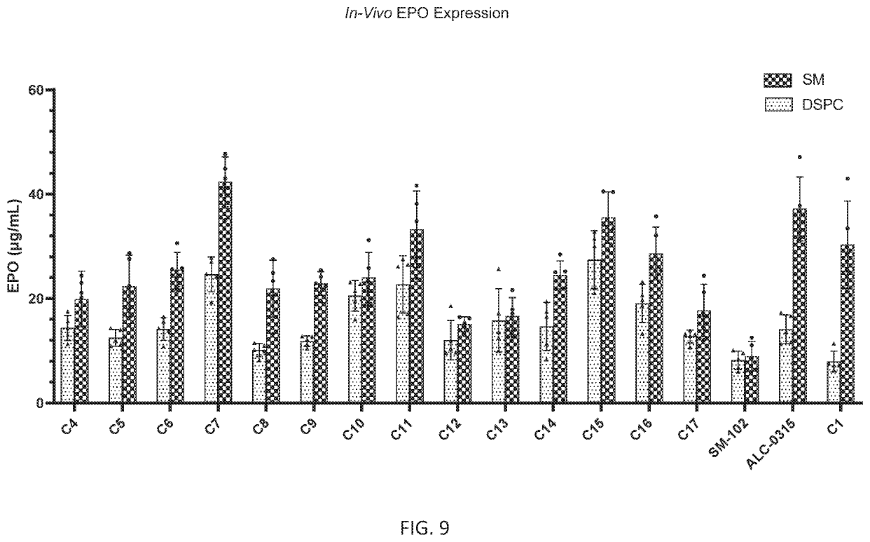

FIGS. 5 and 9 are Cryo-EM images of the present LRNP and reference LNPs that were assessed and characterized using the Cryo-EM analysis described in Example 3 herein. Without being bound by any theory, the microscopic morphology of a sphingomyelin-containing LNP tends to transit towards a higher level of lamellation when the sphingomyelin content in the LNP increases. For example, as shown in FIG. 5 right panel, when sphingomyelin was at about 30 mol % (molar percentage) of the total lipid contents of a LNP formulation, a majority of particles transitioned to a fully lamellar morphology where the lamellar portion fully surrounds the electron-dense core of a particle (arrows), which is sometimes referred to as the “shell-and-core” shape in this application.

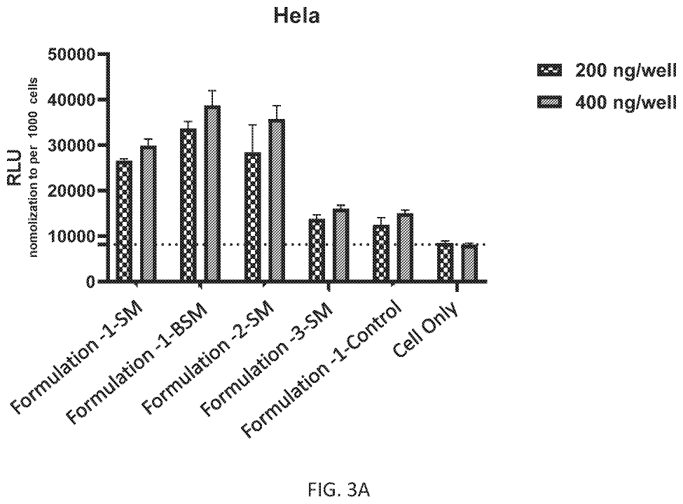

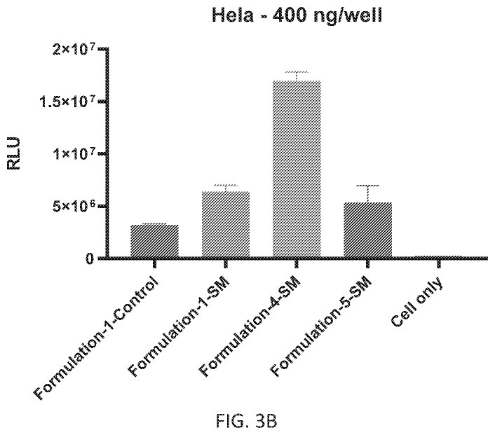

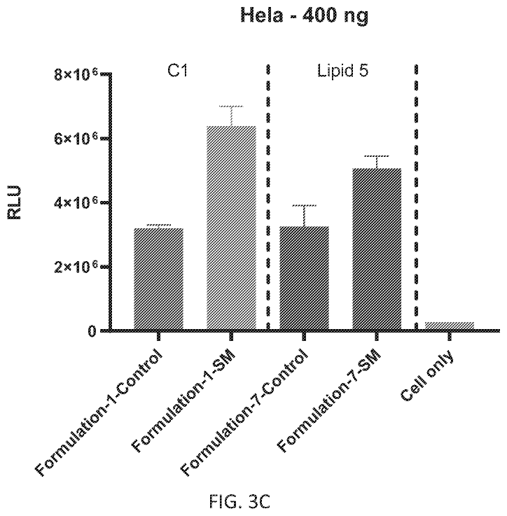

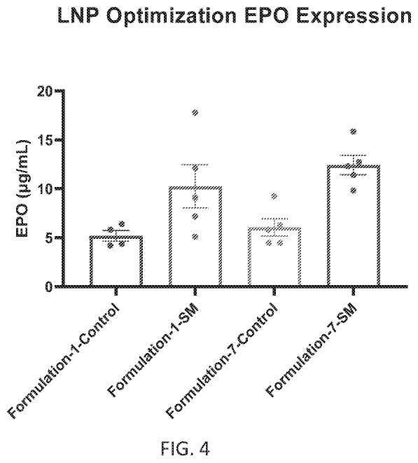

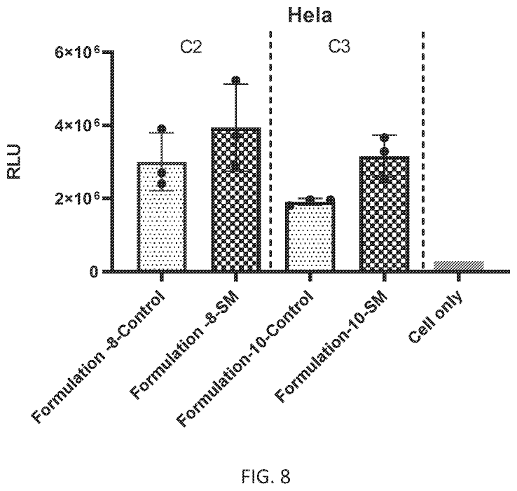

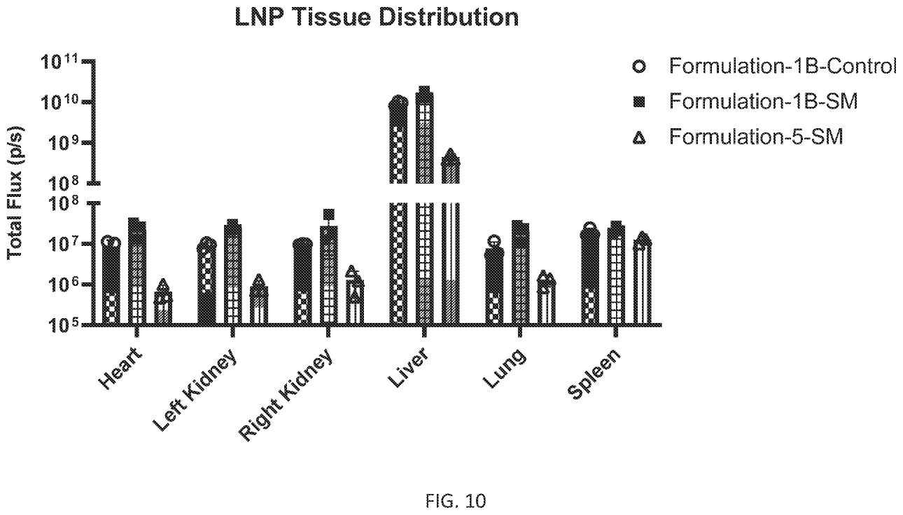

It has been found that the LNP of the present disclosure provide the advantage when used for the in vitro or in vivo delivery of an active agent, such as a therapeutic nucleic acid (e.g., an mRNA). In particular, as illustrated by the Examples herein, the present disclosure provides stable lipid raft nanoparticles (LRNP) that advantageously impart increased activity of the encapsulated nucleic acid (e.g., an mRNA) as compared to nucleic acid-lipid particle compositions previously known. As non-limiting examples, FIGS. 3A to 3C of Example 4, FIG. 4 of Example 5, FIGS. 8 and 9 of Example 8, and FIG. 10 of Example 9 consistently show that in one embodiment of the LRNP disclosed herein, protein expression from an mRNA contained in the present LRNP in a mammalian cell or tissue of a mammal is significantly more than the amount of protein expressed from the same mRNA formulated in a previous described reference nucleic acid-lipid particle composition.

### 5.3.1 Formulations

In one aspect, provided herein are sphingomyelin-containing compositions. In some embodiments, the sphingomyelin-containing compositions described herein are formulated as nanoparticle compositions. Nanoparticle compositions that can be used in connection with the present disclosure include, for example, lipid nanoparticles (LNPs), nano liproprotein particles, liposomes, lipid vesicles, and lipoplexes. In some embodiments, nanoparticle compositions are vesicles including one or more lipid bilayers. In some embodiments, a nanoparticle composition includes two or more concentric bilayers separated by aqueous compartments. Lipid bilayers may be functionalized and/or crosslinked to one another. Lipid bilayers may include one or more ligands, proteins, or channels. In some embodiments, the nanoparticle compositions provided herein are lipid nanoparticles. Lipid nanoparticles comprising nucleic acids and their method of preparation are known in the art, such as those disclosed in, e.g., U.S. Patent Publication No. 2004/0142025, U.S. Patent Publication No. 2007/0042031, PCT Publication No. WO 2017/004143, PCT Publication No. WO 2015/199952, PCT Publication No. WO 2013/016058, and PCT Publication No. WO 2013/086373, the full disclosures of each of which are herein incorporated by reference in their entirety for all purposes. In some embodiments, the largest dimension of a nanoparticle composition provided herein is 1 μm or shorter (e.g., ≤1 μm, ≤900 nm, ≤800 nm, ≤700 nm, ≤600 nm, ≤500 nm, ≤400 nm, ≤300 nm, ≤200 nm, ≤175 nm, ≤150 nm, ≤125 nm, ≤100 nm, ≤75 nm, ≤50 nm, or shorter), such as when measured by dynamic light scattering (DLS), transmission electron microscopy, scanning electron microscopy, or another method. In one embodiment, the lipid nanoparticle provided herein has at least one dimension that is in the range of from about 40 to about 200 nm. In one embodiment, the at least one dimension is in the range of from about 40 to about 100 nm.

In some embodiments, the composition comprises sphingomyelin and at least one lipid that is not sphingomyelin. In some embodiments, the sphingomyelin-containing composition comprises: (a) a sphingomyelin, and (b) a steroid.

In some embodiments, the sphingomyelin-containing comprises (a) a sphingomyelin, (b) a steroid, and further comprises a first lipid component that is not sphingomyelin or steroid. In some embodiments, the first lipid component in the nanoparticle composition comprises (c) a cationic lipid. In some embodiments, the first lipid component in the nanoparticle composition comprises (d) a polymer conjugated lipid. In some embodiments, the first lipid component in the sphingomyelin-containing composition comprises (e) a second phospholipid that is not sphingomyelin. In some embodiments, the first lipid component in the sphingomyelin-containing composition comprises (c) a cationic lipid and (d) a polymer conjugated lipid. In some embodiments, the first lipid component in the sphingomyelin-containing composition comprises (c) a cationic lipid, (d) a polymer conjugated lipid, and (e) a second phospholipid that is not sphingomyelin.

In some embodiment, the sphingomyelin-containing comprises a sphingomyelin, a steroid, a first lipid component that is not sphingomyelin or steroid, and a non-lipid component. In some embodiment, the non-lipid component is a nucleic acid molecule.

In some embodiments, the sphingomyelin-containing composition comprises a sphingomyelin. As used herein and unless otherwise specified, “sphingomyelin” refers to a sphingomyelin compound, or a salt thereof, or a stereoisomer or mixture of stereoisomers thereof. As used herein and unless otherwise specified, a “sphingomyelin compound” provided herein has the following structure:

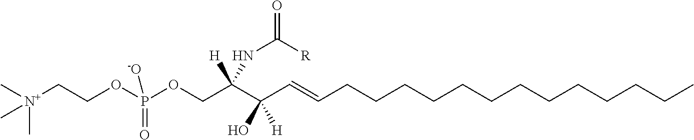

wherein R is an alkyl or alkenyl. As used herein and unless otherwise specified, the descriptions provided herein for sphingomyelin (such as mole ratio) also apply to a sphingomyelin compound, and vice versa, to the extent applicable.

In some embodiments, the sphingomyelin is of about 5 to 40 mol percent of the total lipid present in the composition. In some embodiments, the sphingomyelin is of about 10 to 40 mol percent of the total lipid present in the composition. In some embodiments, the sphingomyelin is of about 10 to 30 mol percent of the total lipid present in the composition. In some embodiments, the sphingomyelin is of about 10 to 25 mol percent of the total lipid present in the composition. In some embodiments, the sphingomyelin is of about 10 to 20 mol percent of the total lipid present in the composition. In some embodiments, the sphingomyelin is of about 10 to 15 mol percent of the total lipid present in the composition. In some embodiments, the sphingomyelin is of about 10 mol percent of the total lipid present in the composition. In some embodiments, the sphingomyelin is of about 15 mol percent of the total lipid present in the composition. In some embodiments, the sphingomyelin is of about 20 mol percent of the total lipid present in the composition.

In some embodiments, the sphingomyelin is of about 5 mol percent, about 6 mol percent, about 7 mol percent, about 8 mol percent, about 9 mol percent, about 10 mol percent, about 11 mol percent, about 11.5 mol percent, about 12 mol percent, about 12.5 mol percent, about 13 mol percent, about 13.5 mol percent, about 14 mol percent, about 14.5 mol percent, about 15 mol percent, about 15.5 mol percent, about 16 mol percent, about 16.5 mol percent, about 17 mol percent, about 17.5 mol percent, about 18 mol percent, about 18.5 mol percent, about 19 mol percent, about 19.5 mol percent, about 20 mol percent, about 21 mol percent, about 22 mol percent, about 23 mol percent, about 24 mol percent, about 25 mol percent, about 30 mol percent, about 35 mol percent, or about 40 mol percent of the total lipid present in the composition.

In some embodiments, the sphingomyelin in the composition is a sphingomyelin compound having the following structure:

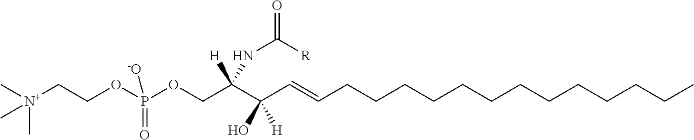

wherein R is an alkyl or alkenyl. In one embodiment, R is a C₁₁-C₂₃ alkyl. In one embodiment, R is a C₁₁-C₁₉ alkyl. In one embodiment, R is a C₁-C₁₉ alkyl. In one embodiment, R is a C₁₅-C₁₉ alkyl. In one embodiment, R is a C₁₁ alkyl (e.g., —(CH₂)₁₀—CH₃). In one embodiment, R is a C₁₃ alkyl (e.g., —(C₁₂)₁₂—CH₃). In one embodiment, R is a C₁₄ alkyl (e.g., —(CH₂)₁₃—CH₃). In one embodiment, R is a C₁₅ alkyl (e.g., —(CH₂)₁₄—CH₃). In one embodiment, R is a C₁₆ alkyl (e.g., (C₁₂)₁₅—CH₃). In one embodiment, R is a C₁₇ alkyl (e.g., —(CH₂)₁₆—CH₃). In one embodiment, R is a C₁₈ alkyl (e.g., —(CH₂)₁₇—CH₃). In one embodiment, R is a C₁₉ alkyl (e.g., —(CH₂)₁₈—CH₃). In one embodiment, R is a C₂₀ alkyl (e.g., —(CH₂)₁₉—CH₃). In one embodiment, R is a C₂₁ alkyl (e.g., —(CH₂)₂₀—CH₃). In one embodiment, R is a C₂₂ alkyl (e.g., —(CH₂)₂₁—CH₃). In one embodiment, R is a C₂₃ alkyl (e.g., —(CH₂)₂₂—CH₃). In one embodiment, the alkyl is a straight alkyl. In one embodiment, the alkyl is a branched alkyl. In one embodiment, the alkyl is unsubstituted. In some embodiments, the sphingomyelin provided herein is selected from the SM-01, SM-02, SM-03, SM-06 and SM-07 molecules shown in Table X below.

In one embodiment, R is a C₁₁-C₂₃ alkenyl. In one embodiment, R is a C₁₃-C₁₉ alkenyl. In one embodiment, R is a C₁₅-C₁₉ alkenyl. In one embodiment, R is a C₁₁ alkenyl. In one embodiment, R is a C₁₃ alkenyl. In one embodiment, R is a C₁₄ alkenyl. In one embodiment, R is a C₁₅ alkenyl. In one embodiment, R is a C₁₆ alkenyl. In one embodiment, R is a C₁₇ alkenyl. In one embodiment, R is a C₁₈ alkenyl. In one embodiment, R is a C₁₉ alkenyl. In one embodiment, R is a C₂₀ alkenyl. In one embodiment, R is a C₂₁ alkenyl. In one embodiment, R is a C₂₂ alkenyl. In one embodiment, R is a C₂₃ alkenyl. In one embodiment, the alkenyl has one double bond. In one embodiment, the double bond has a Z-configuration. In one embodiment, the double bond is at 9-position of the alkenyl R group. In one embodiment, the alkenyl is a straight alkenyl. In one embodiment, the alkenyl is a branched alkenyl. In one embodiment, the alkenyl is unsubstituted. In some embodiments, the sphingomyelin provided herein is selected from SM-04 and SM-05 molecules shown in Table X in Example 7.

In some embodiments, the sphingomyelin-containing composition further comprises a steroid. In some embodiments, the steroid is of about 20 to 50 mol percent of the total lipid present in the composition. In some embodiments, the steroid is of about 30 to 50 mol percent of the total lipid present in the composition. In some embodiments, the steroid is of about 35 to 45 mol percent of the total lipid present in the composition. In some embodiments, the steroid is of about 38.5 to 43.5 mol percent of the total lipid present in the composition. In some embodiments, the steroid is of about 33.5 mol percent of the total lipid present in the composition. In some embodiments, the steroid is of about 38.5 mol percent of the total lipid present in the composition. In some embodiments, the steroid is of about 43.5 mol percent of the total lipid present in the composition.

In some embodiments, the steroid is of about 33.5 mol percent, about 34 mol percent, about 34.5 mol percent, about 35 mol percent, about 35.5 mol percent, about 36 mol percent, about 36.5 mol percent, about 37 mol percent, about 37.5 mol percent, about 38 mol percent, about 38.5 mol percent, about 39 mol percent, about 39.5 mol percent, about 40 mol percent, about 40.5 mol percent, about 41 mol percent, about 41.5 mol percent, about 42 mol percent, about 42.5 mol percent, about 43 mol percent, about 43.5 mol percent, about 44 mol percent, about 44.5 mol percent, about 45 mol percent, about 45.5 mol percent, about 46 mol percent, about 46.5 mol percent, about 47 mol percent, about 47.5 mol percent, about 48 mol percent, or about 48.5 mol percent of the total lipid present in the composition.

In some embodiments, the sphingomyelin-containing composition comprises: (a) a sphingomyelin, and (b) a steroid. In some embodiments, the sphingomyelin is of about 5 to 40 mol percent of the total lipid present in the composition, and the steroid is of about 20 to 50 mol percent of the total lipid present in the composition. In some embodiments, the sphingomyelin is of about 10 to 40 mol percent of the total lipid present in the composition, and the steroid is of about 20 to 50 mol percent of the total lipid present in the composition. In some embodiments, the sphingomyelin is of about 10 to 30 mol percent of the total lipid present in the composition, and the steroid is of about 10 to 25 mol percent of the total lipid present in the composition. In some embodiments, the sphingomyelin is of about 10 to 20 mol percent of the total lipid present in the composition, and the steroid is of about 20 to 50 mol percent of the total lipid present in the composition. In some embodiments, the sphingomyelin is of about 10 to 15 mol percent of the total lipid present in the composition, and the steroid is of about 20 to 50 mol percent of the total lipid present in the composition.

In some embodiments, the sphingomyelin is of about 5 to 40 mol percent of the total lipid present in the composition, and the steroid is of about 33.5 to 43.5 mol percent of the total lipid present in the composition. In some embodiments, the sphingomyelin is of about 10 to 40 mol percent of the total lipid present in the composition, and the steroid is of about 33.5 to 43.5 mol percent of the total lipid present in the composition. In some embodiments, the sphingomyelin is of about 10 to 30 mol percent of the total lipid present in the composition, and the steroid is of about 33.5 to 43.5 mol percent of the total lipid present in the composition. In some embodiments, the sphingomyelin is of about 10 to 20 mol percent of the total lipid present in the composition, and the steroid is of about 33.5 to 43.5 mol percent of the total lipid present in the composition. In some embodiments, the sphingomyelin is of about 10 to 15 mol percent of the total lipid present in the composition, and the steroid is of about 33.5 to 43.5 mol percent of the total lipid present in the composition.

In some embodiments, the sphingomyelin is of about 5 to 40 mol percent of the total lipid present in the composition, and the steroid is of about 30 to 50 mol percent of the total lipid present in the composition. In some embodiments, the sphingomyelin is of about 5 to 40 mol percent of the total lipid present in the composition, and the steroid is of about 35 to 45 mol percent of the total lipid present in the composition. In some embodiments, the sphingomyelin is of about 5 to 40 mol percent of the total lipid present in the composition, and the steroid is of about 33.5 to 43.5 mol percent of the total lipid present in the composition.

In some embodiments, the sphingomyelin is of about 10 to 20 mol percent of the total lipid present in the composition, and the steroid is of about 30 to 50 mol percent of the total lipid present in the composition. In some embodiments, the sphingomyelin is of about 10 to 20 mol percent of the total lipid present in the composition, and the steroid is of about 35 to 45 mol percent of the total lipid present in the composition. In some embodiments, the sphingomyelin is of about 10 to 20 mol percent of the total lipid present in the composition, and the steroid is of about 33.5 to 43.5 mol percent of the total lipid present in the composition.

In some embodiments, the sphingomyelin is of about 10 to 20 mol percent of the total lipid present in the composition, and the steroid is of about 33.5 to 43.5 mol percent of the total lipid present in the composition. In some embodiments, the sphingomyelin is of about 10 mol percent of the total lipid present in the composition, and the steroid is of about 33.5 to 43.5 mol percent of the total lipid present in the composition. In some embodiments, the sphingomyelin is of about 15 mol percent of the total lipid present in the composition, and the steroid is of about 33.5 to 43.5 mol percent of the total lipid present in the composition. In some embodiments, the sphingomyelin is of about 20 mol percent of the total lipid present in the composition, and the steroid is of about 33.5 to 43.5 mol percent of the total lipid present in the composition. In some embodiments, the sphingomyelin is of about 10 to 20 mol percent of the total lipid present in the composition, and the steroid is of about 33.5 mol percent of the total lipid present in the composition. In some embodiments, the sphingomyelin is of about 10 to 20 mol percent of the total lipid present in the composition, and the steroid is of about 38.5 mol percent of the total lipid present in the composition. In some embodiments, the sphingomyelin is of about 10 to 20 mol percent of the total lipid present in the composition, and the steroid is of about 43.5 mol percent of the total lipid present in the composition.

In some embodiment, the steroid in the composition is selected from a steroid described in Section 5.3.5 (Structural Lipids). In some embodiments, the steroid is cholesterol or a cholesterol derivative.

In some embodiments, the sphingomyelin-containing composition comprising (a) the sphingomyelin and (b) the steroid further comprises (c) at least one first lipid component that is not sphingomyelin or steroid.

In some embodiments, the first lipid component comprises (c) a cationic lipid. In some embodiment, the cationic lipid is of about 30 to 55 mol percent of the total lipid present in the composition. In some embodiment, the cationic lipid is of about 35 to 50 mol percent of the total lipid present in the composition. In some embodiment, the cationic lipid is of about 40 to 50 mol percent of the total lipid present in the composition. In some embodiment, the cationic lipid is of about 45 to 50 mol percent of the total lipid present in the composition. In some embodiment, the cationic lipid is of about 40 mol percent of the total lipid present in the composition. In some embodiment, the cationic lipid is of about 45 mol percent of the total lipid present in the composition. In some embodiment, the cationic lipid is of about 50 mol percent of the total lipid present in the composition.

In some embodiments, the cationic lipid is about 35.5 mol percent, about 36 mol percent, about 36.5 mol percent, about 37 mol percent, about 37.5 mol percent, about 38 mol percent, about 38.5 mol percent, about 39 mol percent, about 39.5 mol percent, about 40 mol percent, about 40.5 mol percent, about 41 mol percent, about 41.5 mol percent, about 42 mol percent, about 42.5 mol percent, about 43 mol percent, about 43.5 mol percent, about 44 mol percent, about 44.5 mol percent of the total lipid present in the nanoparticle composition.

In some embodiments, the sphingomyelin-containing composition comprises: (a) a sphingomyelin, (b) a steroid; and (c) a cationic lipid. In some embodiments, the sphingomyelin is of about 5 to 40 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 10 to 30 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 10 to 25 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 10 to 20 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 10 to 15 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 10 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 15 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 20 mol percent of the total lipid present in the composition, the steroid is of about 20 to 50 mol percent of the total lipid present in the composition; and the cationic lipid is about 30 to 55 mol percent of the total lipid present in the composition.

In some embodiments, the sphingomyelin is of about 5 to 40 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 30 to 50 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 35 to 45 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 33.5 to 43.5 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 33.5 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 38.5 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 43.5 mol percent of the total lipid present in the composition; and the cationic lipid is about 30 to 55 mol percent of the total lipid present in the composition.

In some embodiments, the sphingomyelin is of about 5 to 40 mol percent of the total lipid present in the composition, the steroid is of about 20 to 50 mol percent of the total lipid present in the composition; and the cationic lipid is about 30 to 55 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 35 to 50 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 40 to 50 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 45 to 50 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 40 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 45 mol percent of the total lipid present in the composition. In some embodiments, the sphingomyelin is of about 5 to 40 mol percent of the total lipid present in the composition, the steroid is of about 20 to 50 mol percent of the total lipid present in the composition; and the cationic lipid is about 50 mol percent of the total lipid present in the composition.

In some embodiments, the sphingomyelin is of about 10 to 20 mol percent of the total lipid present in the composition, the steroid is of about 40 to 50 mol percent of the total lipid present in the composition; and the cationic lipid is about 33.5 to 43.5 mol percent of the total lipid present in the composition. In some embodiments, the sphingomyelin is of about 10 mol percent of the total lipid present in the composition, the steroid is of about 40 to 50 mol percent of the total lipid present in the composition; and the cationic lipid is about 33.5 to 43.5 mol percent of the total lipid present in the composition. In some embodiments, the sphingomyelin is of about 15 mol percent of the total lipid present in the composition, the steroid is of about 40 to 50 mol percent of the total lipid present in the composition; and the cationic lipid is about 33.5 to 43.5 mol percent of the total lipid present in the composition. In some embodiments, the sphingomyelin is of about 20 mol percent of the total lipid present in the composition, the steroid is of about 40 to 50 mol percent of the total lipid present in the composition; and the cationic lipid is about 33.5 to 43.5 mol percent of the total lipid present in the composition. In some embodiments, the sphingomyelin is of about 10 to 20 mol percent of the total lipid present in the composition, the steroid is of about 40 mol percent of the total lipid present in the composition; and the cationic lipid is about 33.5 to 43.5 mol percent of the total lipid present in the composition. In some embodiments, the sphingomyelin is of about 10 to 20 mol percent of the total lipid present in the composition, the steroid is of about 45 mol percent of the total lipid present in the composition; and the cationic lipid is about 33.5 to 43.5 mol percent of the total lipid present in the composition. In some embodiments, the sphingomyelin is of about 10 to 20 mol percent of the total lipid present in the composition, the steroid is of about 50 mol percent of the total lipid present in the composition; and the cationic lipid is about 33.5 to 43.5 mol percent of the total lipid present in the composition. In some embodiments, the sphingomyelin is of about 10 to 20 mol percent of the total lipid present in the composition, the steroid is of about 40 to 50 mol percent of the total lipid present in the composition; and the cationic lipid is about 33.5 mol percent of the total lipid present in the composition. In some embodiments, the sphingomyelin is of about 10 to 20 mol percent of the total lipid present in the composition, the steroid is of about 40 to 50 mol percent of the total lipid present in the composition; and the cationic lipid is about 38.5 mol percent of the total lipid present in the composition. In some embodiments, the sphingomyelin is of about 10 to 20 mol percent of the total lipid present in the composition, the steroid is of about 40 to 50 mol percent of the total lipid present in the composition; and the cationic lipid is about 43.5 mol percent of the total lipid present in the composition.

In some embodiments, the cationic lipid is a lipid compound as described in Section Sections 5.3.2 (Cationic Lipids) herein. In some embodiments, the cationic lipid is a compound according to any one of the formulae selected from Formula 01-I, 01-II, 02-I, 02-II, 03-I, 03-II-A, 03-II-B, 03-II-C, 03-II-D, 04-I, 04-III, 04-IV, 05-I, 06-I, and sub-formulas thereof described herein. In some embodiments, the cationic lipid is a compound selected from the compounds listed in Tables 1 to 5. In some embodiments, the cationic lipid is a compound selected from Table Y of Example 8.

In some embodiments, the first lipid component comprises a polymer conjugated lipid. In some embodiment, the polymer conjugated lipid is of about 0.5 to 3 mol percent of the total lipid present in the composition. In some embodiment, the polymer conjugated lipid is of about 0.5 mol percent, about 0.6 mol percent, about 0.7 mol percent, about 0.8 mol percent, about 0.9 mol percent, about 1 mol percent, about 1.1 mol percent, about 1.2 mol percent, about 1.3 mol percent, about 1.4 mol percent, about 1.5 mol percent, about 1.6 mol percent, about 1.7 mol percent, about 1.8 mol percent, about 1.9 mol percent, about 2 mol percent, about 2.1 mol percent, about 2.2 mol percent, about 2.3 mol percent, about 2.4 mol percent, about 2.5 mol percent, about 2.6 mol percent, about 2.7 mol percent, about 2.8 mol percent, about 2.9 mol percent, or about 3 mol percent of the total lipid present in the composition.

In some embodiments, the sphingomyelin-containing composition comprises: (a) a sphingomyelin, (b) a steroid, (c) a cationic lipid, and (d) a polymer conjugated lipid. In some embodiments, the sphingomyelin is of about 5 to 40 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 10 to 30 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 10 to 25 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 10 to 20 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 10 to 15 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 10 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 15 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 20 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition.

In some embodiments, the sphingomyelin is of about 5 to 40 mol percent of the total lipid present in the composition, the steroid is of about 20 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 30 to 50 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 35 to 45 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 33.5 to 43.5 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 33.5 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 38.5 mol percent of the total lipid present in the composition; the cationic lipid is about 30 to 55 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 43.5 mol percent of the total lipid present in the composition; the cationic lipid is about 30 to 55 mol percent of the total lipid present in the composition; and the polymer conjugated lipid is about 0.5 to 3 mol percent of the total lipid present in the composition.

In some embodiments, the sphingomyelin is of about 5 to 40 mol percent of the total lipid present in the composition, the steroid is of about 20 to 50 mol percent of the total lipid present in the composition; the cationic lipid is about 35 to 50 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 20 to 50 mol percent of the total lipid present in the composition; the cationic lipid is about 40 to 50 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 20 to 50 mol percent of the total lipid present in the composition; the cationic lipid is about 45 to 50 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 20 to 50 mol percent of the total lipid present in the composition; the cationic lipid is about 40 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 20 to 50 mol percent of the total lipid present in the composition; the cationic lipid is about 45 mol percent of the total lipid present in the composition; and the polymer conjugated lipid is about 0.5 to 3 mol percent of the total lipid present in the composition. In some embodiments, the sphingomyelin is of about 5 to 40 mol percent of the total lipid present in the composition, the steroid is of about 20 to 50 mol percent of the total lipid present in the composition; the cationic lipid is about 50 mol percent of the total lipid present in the composition; and the polymer conjugated lipid is about 0.5 to 3 mol percent of the total lipid present in the composition.

In some embodiments, the sphingomyelin is of about 10 to 20 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 0.5 to 3 mol percent of the total lipid present in the composition.

In some embodiments, the sphingomyelin is of about 10 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 0.5 to 3 mol percent of the total lipid present in the composition. In some embodiments, the sphingomyelin is of about 15 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 0.5 to 3 mol percent of the total lipid present in the composition. In some embodiments, the sphingomyelin is of about 20 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 0.5 to 3 mol percent of the total lipid present in the composition.

In some embodiments, the sphingomyelin is of about 10 to 20 mol percent of the total lipid present in the composition, the steroid is of about 33.5 mol percent of the total lipid present in the composition; the cationic lipid is about 40 to 50 mol percent of the total lipid present in the composition; and the polymer conjugated lipid is about 0.5 to 3 mol percent of the total lipid present in the composition. In some embodiments, the sphingomyelin is of about 10 to 20 mol percent of the total lipid present in the composition, the steroid is of about 38.5 mol percent of the total lipid present in the composition; the cationic lipid is about 40 to 50 mol percent of the total lipid present in the composition; and the polymer conjugated lipid is about 0.5 to 3 mol percent of the total lipid present in the composition. In some embodiments, the sphingomyelin is of about 10 to 20 mol percent of the total lipid present in the composition, the steroid is of about 43.5 mol percent of the total lipid present in the composition; the cationic lipid is about 40 to 50 mol percent of the total lipid present in the composition; and the polymer conjugated lipid is about 0.5 to 3 mol percent of the total lipid present in the composition.

In some embodiments, the sphingomyelin is of about 10 to 20 mol percent of the total lipid present in the composition, the steroid is of about 33.5 to 43.5 mol percent of the total lipid present in the composition; the cationic lipid is about 40 mol percent of the total lipid present in the composition; and the polymer conjugated lipid is about 0.5 to 3 mol percent of the total lipid present in the composition. In some embodiments, the sphingomyelin is of about 10 to 20 mol percent of the total lipid present in the composition, the steroid is of about 33.5 to 43.5 mol percent of the total lipid present in the composition; the cationic lipid is about 45 mol percent of the total lipid present in the composition; and the polymer conjugated lipid is about 0.5 to 3 mol percent of the total lipid present in the composition. In some embodiments, the sphingomyelin is of about 10 to 20 mol percent of the total lipid present in the composition, the steroid is of about 33.5 to 43.5 mol percent of the total lipid present in the composition; the cationic lipid is about 50 mol percent of the total lipid present in the composition; and the polymer conjugated lipid is about 0.5 to 3 mol percent of the total lipid present in the composition.

In some embodiments, the sphingomyelin is of about 10 to 20 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 0.5 mol percent of the total lipid present in the composition. En some embodiments, the sphingomyelin is of about 10 to 20 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 1.5 mol percent of the total lipid present in the composition. In some embodiments, the sphingomyelin is of about 10 to 20 mol percent of the total lipid present in the composition, the steroid is of about 33.5 to 43.5 mol percent of the total lipid present in the composition; the cationic lipid is about 40 to 50 mol percent of the total lipid present in the composition; and the polymer conjugated lipid is about 3 mol percent of the total lipid present in the composition.

In some embodiments, the sphingomyelin is about 10 mol percent of the total lipid present in the composition; wherein the cationic lipid is about 50 mol percent of the total lipid present in the composition; wherein the steroid is about 38.5 mol percent of the total lipid present in the composition; and wherein the polymer conjugated lipid is about 1.5 mol percent of the total lipid present in the composition.

In some embodiments, the sphingomyelin is about 10 mol percent of the total lipid present in the composition; the cationic lipid is about 45 mol percent of the total lipid present in the composition; the steroid is about 43.5 mol percent of the total lipid present in the composition; and the polymer conjugated lipid is about 1.5 mol percent of the total lipid present in the composition.

In some embodiments, the sphingomyelin is about 10 mol percent of the total lipid present in the composition; the cationic lipid is about 40 mol percent of the total lipid present in the composition; the steroid is about 48.5 mol percent of the total lipid present in the composition; and the polymer conjugated lipid is about 1.5 mol percent of the total lipid present in the composition.

In some embodiments, the sphingomyelin is about 15 mol percent of the total lipid present in the composition; the cationic lipid is about 45 mol percent of the total lipid present in the composition; the steroid is about 38.5 mol percent of the total lipid present in the composition; and the polymer conjugated lipid is about 1.5 mol percent of the total lipid present in the composition.

In some embodiments, the sphingomyelin is about 15 mol percent of the total lipid present in the composition; the cationic lipid is about 40 mol percent of the total lipid present in the composition; the steroid is about 43.5 mol percent of the total lipid present in the composition; and the polymer conjugated lipid is about 1.5 mol percent of the total lipid present in the composition.

In some embodiments, the sphingomyelin is about 20 mol percent of the total lipid present in the composition; the cationic lipid is about 45 mol percent of the total lipid present in the composition; the steroid is about 33.5 mol percent of the total lipid present in the composition; and the polymer conjugated lipid is about 1.5 mol percent of the total lipid present in the composition.

In some embodiments, the sphingomyelin is about 20 mol percent of the total lipid present in the composition; the cationic lipid is about 40 mol percent of the total lipid present in the composition; the steroid is about 38.5 mol percent of the total lipid present in the composition; and the polymer conjugated lipid is about 1.5 mol percent of the total lipid present in the composition.

In some embodiments, the sphingomyelin is about 5 mol percent of the total lipid present in the composition; the cationic lipid is about 45 mol percent of the total lipid present in the composition; the steroid is about 48.5 mol percent of the total lipid present in the composition; and the polymer conjugated lipid is about 1.5 mol percent of the total lipid present in the composition.

In some embodiments, the sphingomyelin is about 5 mol percent of the total lipid present in the composition; the cationic lipid is about 45 mol percent of the total lipid present in the composition; the steroid is about 48.5 mol percent of the total lipid present in the composition; and the polymer conjugated lipid is about 1.5 mol percent of the total lipid present in the composition.

In some embodiments, the polymer conjugated lipid is selected from a lipid described in Section 5.3.4 (Polymer Conjugated Lipids) herein. In some embodiments, the polymer conjugated lipid is DMG-PEG. In some embodiments, the polymer conjugated lipid is DMG-PEG2000 or DMPE-PEG2000.

In some embodiments, the first lipid component comprises a second phospholipid that is not sphingomyelin. In some embodiment, the total phospholipid content (inclusive of sphingomyelin and the second phospholipid) is about 5 to 40 mol percent of the total lipid present in the composition. In some embodiment, the total phospholipid content (inclusive of sphingomyelin and the second phospholipid) is about 10 to 30 mol percent of the total lipid present in the composition. In some embodiment, the total phospholipid content (inclusive of sphingomyelin and the second phospholipid) is about 10 to 20 mol percent of the total lipid present in the composition. In some embodiment, the total phospholipid content (inclusive of sphingomyelin and the second phospholipid) is about 10 to 15 mol percent of the total lipid present in the composition. In some embodiment, the total phospholipid content (inclusive of sphingomyelin and the second phospholipid) is about 10 mol percent of the total lipid present in the composition. In some embodiment, the total phospholipid content (inclusive of sphingomyelin and the second phospholipid) is about 15 mol percent of the total lipid present in the composition. In some embodiment, the total phospholipid content (inclusive of sphingomyelin and the second phospholipid) is about 20 mol percent of the total lipid present in the composition. In some embodiments, the molar ratio between sphingomyelin and the second phospholipid is about 1:3 to 3:1. In some embodiments, the molar ratio between sphingomyelin and the second phospholipid is about 1:3. In some embodiments, the molar ratio between sphingomyelin and the second phospholipid is about 1:1. In some embodiments, the molar ratio between sphingomyelin and the second phospholipid is about 3:1.

In some embodiments, the sphingomyelin-containing composition comprises: (a) a sphingomyelin, (b) a steroid, (c) a cationic lipid, (d) a polymer-conjugated lipid, and (e) a second phospholipid that is not sphingomyelin. In some embodiments, the total phospholipid content (inclusive of sphingomyelin and the second phospholipid) is of about 5 to 40 mol percent of the total lipid present in the composition, the steroid is of about 20 to 50 mol percent of the total lipid present in the composition; the cationic lipid is about 30 to 55 mol percent of the total lipid present in the composition; the polymer conjugated lipid is about 0.5 to 3 mol percent of the total lipid present in the composition, and the molar ratio between sphingomyelin and the second phospholipid is about 1:3 to 3:1.

In some embodiments, the total phospholipid content (inclusive of sphingomyelin and the second phospholipid) is of about 5 to 20 mol percent of the total lipid present in the composition, the steroid is of about 33.5 to 43.5 mol percent of the total lipid present in the composition; the cationic lipid is about 40 to 50 mol percent of the total lipid present in the composition; the polymer conjugated lipid is about 0.5 to 3 mol percent of the total lipid present in the composition, and the molar ratio between sphingomyelin and the second phospholipid is about 1:3 to 3:1. In some embodiments, the molar ratio between sphingomyelin and the second phospholipid is about 1:1.

In some embodiments, the total phospholipid content (inclusive of sphingomyelin and the second phospholipid) is of about 5 mol percent of the total lipid present in the composition, the steroid is of about 33.5 to 43.5 mol percent of the total lipid present in the composition; the cationic lipid is about 40 to 50 mol percent of the total lipid present in the composition; the polymer conjugated lipid is about 0.5 to 3 mol percent of the total lipid present in the composition, and the molar ratio between sphingomyelin and the second phospholipid is about 1:3 to 3:1. In some embodiments, the molar ratio between sphingomyelin and the second phospholipid is about 1:1.

In some embodiments, the total phospholipid content (inclusive of sphingomyelin and the second phospholipid) is of about 15 mol percent of the total lipid present in the composition, the steroid is of about 33.5 to 43.5 mol percent of the total lipid present in the composition; the cationic lipid is about 40 to 50 mol percent of the total lipid present in the composition; the polymer conjugated lipid is about 0.5 to 3 mol percent of the total lipid present in the composition, and the molar ratio between sphingomyelin and the second phospholipid is about 1:3 to 3:1. In some embodiments, the molar ratio between sphingomyelin and the second phospholipid is about 1:1.

In some embodiments, the total phospholipid content (inclusive of sphingomyelin and the second phospholipid) is of about 20 mol percent of the total lipid present in the composition, the steroid is of about 33.5 to 43.5 mol percent of the total lipid present in the composition; the cationic lipid is about 40 to 50 mol percent of the total lipid present in the composition; the polymer conjugated lipid is about 0.5 to 3 mol percent of the total lipid present in the composition, and the molar ratio between sphingomyelin and the second phospholipid is about 1:3 to 3:1. In some embodiments, the molar ratio between sphingomyelin and the second phospholipid is about 1:1.

In some embodiments, the sphingomyelin is about 5 mol percent of the total lipid present in the composition; the cationic lipid is about 45 mol percent of the total lipid present in the composition; the steroid is about 43.5 mol percent of the total lipid present in the composition; and the polymer conjugated lipid is about 1.5 mol percent of the total lipid present in the composition; and wherein the composition further comprises a second phospholipid of about 5 mol percent of the total lipid present in the composition.

In some embodiments, the second phospholipid is a compound selected from Section 5.3.6 (Phospholipids) herein. In some embodiments, the second phospholipid is DSPC.

In a related aspect, the present disclosure is based, at least in part, upon the discovery that nucleic acid containing lipid nanoparticles that comprises sphingomyelin as a structural component can result in an enhanced level of expression of the nucleic acid molecule comparing to reference nucleic acid containing lipid nanoparticles that do not contain sphingomyelin. Without being bound by the theory, it is contemplated that these lipid nanoparticles contain sphingomyelin-rich liquid-ordered (Lo) domains (rafts) that disperse in liquid disordered (Id) non-raft regions formed by other lipid components. In some embodiments, these lipid nanoparticles exhibit a semi-lamellar morphology under electronic microscopy such as illustrated in FIG. 2B.

In some embodiments, the sphingomyelin-containing composition comprises: (a) a sphingomyelin, (b) a steroid, (c) a cationic lipid, (d) a polymer-conjugated lipid, and (e) non-lipid component. In some embodiments, the non-lipid component comprises a therapeutic agent. In some embodiments, the therapeutic agent is a molecule as described in Section 5.3.7 (Therapeutic Payload) herein. In some embodiments, the non-lipid component is a nucleic acid molecule. In some embodiments, the non-lipid component is an mRNA molecule.

In some embodiments, the sphingomyelin-containing compositions described herein are formulated as nanoparticle compositions. According to the present disclosure, any of the sphingomyelin-containing compositions described herein can be formulated as a nanoparticle composition. Any of the content(s), composition(s), and/or lipid molar ratio(s) or percentage(s) as described herein for any of the sphingomyelin-containing compositions also apply to the nanoparticle compositions, mutatis mutandis. For illustrative and non-limiting purposes only, a description of a sphingomyelin-containing composition herein such as the sphingomyelin-containing composition comprises a sphingomyelin of about 10 mol percent of the total lipid present in the sphingomyelin-containing composition, when applied to a nanoparticle composition would be that the nanoparticle composition comprises a sphingomyelin of about 10 mol percent of the total lipid present in the nanoparticle composition.

In some embodiments, the sphingomyelin-containing compositions described herein are formulated as lipid nanoparticle compositions. In some embodiments, the nanoparticle composition comprises a plurality of lipid nanoparticles. Accordingly, any of the content(s), composition(s), and/or lipid molar ratio(s) or percentage(s) as described herein for any of the sphingomyelin-containing compositions also apply to the nanoparticles (including LRNPs) in a nanoparticle composition, mutatis mutandis. For illustrative and non-limiting purposes only, a description of a sphingomyelin-containing composition herein such as the sphingomyelin-containing composition comprises a sphingomyelin of about 10 mol percent of the total lipid present in the sphingomyelin-containing composition, when applied to one or more lipid nanoparticles would be that the one or more lipid nanoparticles comprise a sphingomyelin of about 10 mol percent of the total lipid present in the one or more lipid nanoparticles.

In some embodiments, the nanoparticle composition comprises a plurality of nanoparticles that comprise a nucleic acid molecule. In some embodiments, the nanoparticle composition comprises a plurality of nanoparticles enclosing the nucleic acid molecule within a lipid shell. In some embodiments, the lipid shells protect the nucleic acid molecules from degradation. In some embodiments, the nanoparticles also facilitate transportation of the enclosed nucleic acid molecules into intracellular compartments and/or machinery to exert an intended therapeutic of prophylactic function. In certain embodiments, nucleic acids, when present in the lipid nanoparticles, are resistant in aqueous solution to degradation with a nuclease.

In some embodiments, the nanoparticle composition comprises (a) the sphingomyelin and (b) the steroid, as described herein. In some embodiments, the nanoparticle composition comprises (a) the sphingomyelin, (b) the steroid, and further comprises the first lipid component that is not sphingomyelin or steroid, as described herein. In some embodiments, the first lipid component in the nanoparticle composition comprises (c) the cationic lipid, as described herein. In some embodiments, the first lipid component in the nanoparticle composition comprises (c) the cationic lipid and (d) the polymer conjugated lipid, as described herein. In some embodiments, the first lipid component in the nanoparticle composition comprises (c) the cationic lipid, (d) the polymer conjugated lipid, and further comprises (e) a second phospholipid that is not sphingomyelin, as described herein.

In some embodiments, the nanoparticle composition comprises a plurality of nanoparticles. In some embodiments, one or more nanoparticles in the composition has a heterogeneous structure comprising at least one liquid-ordered (Lo) domain and at least one liquid-disordered (Ld) region. In some embodiments, the at least one Lo domains disperse in the Ld region in the nanoparticle.

In some embodiments, the nanoparticle comprises (a) sphingomyelin, (b) steroid, and the first lipid component that is not sphingomyelin or steroid. In some embodiments, the Lo domain comprises sphingomyelin. In some embodiments, the Lo domain is higher in the sphingomyelin concentration as comparing to the Ld region of the nanoparticle. In some embodiments, the Lo domain comprises the steroid. In some embodiments, the Lo domain is higher in the steroid concentration as comparing to the Ld region of the nanoparticle. In some embodiment, the Ld region comprises the first lipid component. In some embodiment, the Ld region contains higher concentration of the first lipid component as comparing to the Lo domain. In some embodiments, the first lipid component comprises a cationic lipid. In some embodiments, the first lipid component comprises a polymer conjugated lipid. In some embodiments, the first lipid component comprises a second phospholipid that is not sphingomyelin. In some embodiments, the first lipid component comprises a cationic lipid and a polymer conjugated lipid. In some embodiments, the first lipid component comprises a cationic lipid, a polymer conjugated lipid, and a second phospholipid that is not sphingomyelin.

In some embodiments, the nanoparticle comprises is a lipid raft nanoparticle (LRNP) as described herein and comprises one or more liquid-ordered (Lo) domains disperse in at least one liquid-disordered (Ld) region. In some embodiments, the Lo domain of the LRNP comprises lipid rafts. In some embodiments, the Lo domain of the LRNP comprises sphingomyelin and steroid. In some embodiments, the Lo domain of the LRNP comprises sphingomyelin and steroid that are bond to one another through the hydrogen bond as depicted in FIG. 1B.

In some embodiments, the Ld region of the nanoparticle is electron dense under electron microscopy. In some embodiments, the Lo domain of the nanoparticle is not electron dense under electron microscopy. In some embodiments, the Lo domain of the nanoparticle assumes a uni-lamellar structure under electron microscopy. In some embodiments, the Lo domain of the nanoparticle assumes a multi-lamellar structure under electron microscopy. In some embodiments, the nanoparticle comprises one electron dense core. In some embodiments, the nanoparticle comprises one lamellar portion surrounding less than the entire electron dense core. In some embodiments, the nanoparticle comprises two or more lamellar portions that collectively surround less than of the entire electron dense core. In some embodiments, the nanoparticle assumes a semi-lamellar morphology under electron microscopy, wherein the nanoparticle comprises an electron-dense core and at least one lamellar portion, and wherein the lamellar portion surrounds less than the entire electron-dense core. In some embodiments, the electron microscopy is Cryo-transmission electron microscopy (“Cryo-TEM”), electron cryomicroscopy (“Cryo-EM”), differential scanning calorimetry (“DSC”), or x-ray diffraction. In some embodiments, the electron microscopy is Cryo-TEM.

In some embodiments, the nanoparticle composition comprises a plurality of nanoparticles, wherein at least 10%, 20%, 30%, 40%, 50%, 60%, 70%, 80%, 90% or 95% of the plurality of nanoparticles have a semi-lamellar morphology. In some embodiments, at least 55% of the plurality of nanoparticles in the nanoparticle composition according to the present disclosure have a semi-lamellar morphology. In some embodiments, the semi-lamellar morphology of the nanoparticle is a microscopic morphology visible under electron microscopy. In some embodiments, the electron microscopy is Cryo-transmission electron microscopy (“Cryo-TEM”), electron cryomicroscopy (“Cryo-EM”), differential scanning calorimetry (“DSC”), or x-ray diffraction. In some embodiments, the electron microscopy is Cryo-TEM.

In various embodiments of the present disclosure, the nanoparticle composition further comprises a non-lipid component. In some embodiments, a plurality of nanoparticles in the nanoparticle composition comprises the non-lipid component. In some embodiments, the non-lipid component comprises a therapeutic agent. In some embodiments, the non-lipid component is a molecule as described in Section 5.3.7 (Therapeutic Payload) herein. In some embodiments, the non-lipid component is a nucleic acid molecule. In some embodiments, the non-lipid component is an mRNA molecule.

In some embodiments, the nanoparticles in s comprises a nucleic acid. In some embodiments, the nucleic acid encodes an RNA or a protein. In some embodiments, the amount of RNA or protein expressed from the nucleic acid in the nanoparticle is more than the amount of RNA or protein expressed from the nucleic acid formulated in a nucleic acid-lipid reference nanoparticle composition (reference nanoparticle composition).

In some embodiments, the reference nanoparticle composition does not contain sphingomyelin. In some embodiments, the reference nanoparticle composition does not contain sphingomyelin of about 5 to 40 mol percent of the total lipid present in the particle. In some embodiments, the reference nanoparticle composition does not contain sphingomyelin of about 10 to 30 mol percent of the total lipid present in the particle. In some embodiments, the reference nanoparticle composition does not contain sphingomyelin of about 10 to 25 mol percent of the total lipid present in the particle. In some embodiments, the reference nanoparticle composition does not contain sphingomyelin of about 10 to 20 mol percent of the total lipid present in the particle. In some embodiments, the reference nanoparticle composition does not contain sphingomyelin of about 10 to 15 mol percent of the total lipid present in the particle. In some embodiments, the reference nanoparticle composition does not contain sphingomyelin of about 10 mol percent of the total lipid present in the particle. In some embodiments, the reference nanoparticle composition does not contain sphingomyelin of about 15 mol percent of the total lipid present in the particle. In some embodiments, the reference nanoparticle composition does not contain sphingomyelin of about 20 mol percent of the total lipid present in the particle.

In some embodiments, the reference nanoparticle composition contains a second lipid instead of sphingomyelin. In some embodiments, wherein the molar percentage of the second lipid in the total lipid present in the reference nanoparticle composition is the same as the molar percentage of sphingomyelin in the total lipid present in the nanoparticle composition, for which the reference nanoparticle composition is used as a reference (e.g., for comparative studies). In some embodiments, the reference nanoparticle composition has the same composition as the nanoparticle composition except that sphingomyelin is replaced by a second lipid of equal molar percentage in the reference nanoparticle composition. In some embodiments, the second lipid is a phospholipid. In some embodiments, the second lipid is DSPC.

In some embodiments, upon delivery of the nucleic acid-containing nanoparticle composition to a host cell, the nucleic acid is expressed to form RNA and/or protein via the host cell endogenous transcription and/or translation machinery. In some embodiments, upon delivery of the nucleic acid-containing nanoparticle composition to a host cell, the expression level of the nucleic acid formulated in the present sphingomyelin-containing nanoparticle composition is enhanced as compared to the nucleic acid formulated in a reference nanoparticle composition as described herein. In some embodiments, the reference nanoparticle composition comprises the same composition except that the sphingomyelin is replaced by another phospholipid of equal molar percentage. In some embodiments, the other phospholipid is 1,2-distearoyl-sn-glycero-3-phosphocholine (DSPC). In some embodiments, the molar percentage of sphingomyelin in the total lipid present in the sphingomyelin-containing nanoparticle composition according to the present disclosure is the same as the molar percentage of DSPC in the total lipid present in the reference nanoparticle composition. In some embodiments, the reference nanoparticle composition does not comprise sphingomyelin.

In some embodiments, expression level of the nucleic acid molecule formulated in the present sphingomyelin containing nanoparticle composition is increased at least 10%, 20%, 30%, 40%, 50%, 60%, 70%, 80%, or 90% compared to the expression level of the nucleic acid formulated in the reference nanoparticle composition. In some embodiments, expression level of the nucleic acid is measured as the amount of RNA or protein encoded by the nucleic acid that is produced by the host cell. In some embodiments, the host cell is a mammalian cell, such as a cell from human or a non-human vertebrate.

Accordingly, the nanoparticle composition according to the present disclosure can be used in a method of expressing a nucleic acid molecule (e.g., DNA or RNA) in a host cell or tissue of a host subject, wherein the method comprises formulating the nucleic acid molecule within a sphingomyelin-containing nanoparticle composition according to the present disclosure, and delivering the nanoparticle composition to the host cells or the host subject; and wherein the delivered nucleic acid molecule is expressed in the host cell or in the host subject. In some embodiments, the host cell is a mammalian cell (such as a cell originated from human or a non-human vertebrate). In some embodiments, the host subject is a mammal (such as human or non-human vertebrate). In some embodiments, delivering the nanoparticle composition can be performed by contacting the nanoparticle composition in vitro with the host cells. In some embodiments, delivering the nanoparticle composition can be performed by administering the nanoparticle composition in vivo to the host subject. In some embodiments, the nucleic acid encodes for a RNA, peptide or polypeptide. In some embodiments, the nucleic acid molecule encodes an RNA that is not an mRNA. In some embodiments, the nucleic acid molecule encodes an RNA that is an mRNA. In some embodiments, the nucleic acid molecule is DNA. In some embodiments, the nucleic acid molecule is RNA. In some embodiments, the delivered nucleic acid molecule is mRNA.

Accordingly, in a related aspect of the present disclosure, provided herein are methods for expressing a nucleic acid molecule, wherein the method comprises formulating the nucleic acid molecule within a sphingomyelin-containing nanoparticle composition according to the present disclosure, and delivering the nanoparticle composition to a host cell, and wherein the delivered nucleic acid molecule is expressed in the host cell. In some embodiments, the host cell is isolated and the delivery is performed by contacting the nanoparticle composition with the host cell under a suitable condition in vitro, wherein the nucleic acid molecule is expressed by the host cell. In some embodiments, the host cell is in its native environment is a subject, and the delivery is performed by administering a suitable amount of the nanoparticle composition to the subject, wherein the nucleic acid molecule is expressed the host cell in the subject. In some embodiments, the nucleic acid molecule encodes for a RNA, peptide or polypeptide. In some embodiments, the nucleic acid molecule encodes an RNA that is not an mRNA. In some embodiments, the nucleic acid molecule encodes an RNA that is an mRNA. In some embodiments, the nucleic acid molecule is DNA. In some embodiments, the nucleic acid molecule is RNA. In some embodiments, the nucleic acid molecule is mRNA.

According to the present disclosure, nanoparticle compositions described herein can include at least one lipid component and one or more additional components, such as a therapeutic and/or prophylactic agent (e.g., the therapeutic nucleic acid described herein). A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

In some embodiments, the therapeutic and/or prophylactic agent encapsulated in the nanoparticles can be delivered to a host cell in vitro, for example, by contacting the host cell with the nanoparticle composition, or in vivo, for example, by administering the nanoparticle composition to a subject containing the host cell. In some embodiments, upon delivery, the therapeutic nucleic acid molecule encapsulated in the nanoparticle can be expressed via the host cell endogenous transcription and translation machinery.

In some embodiment, the therapeutic agent to lipid ratio in the nanoparticle composition (i.e., N/P, were N represents the moles of cationic lipid and P represents the moles of phosphate present as part of the nucleic acid backbone) range from 2:1 to 30:1, for example 3:1 to 22:1. In one embodiment, N/P ranges from 6:1 to 20:1 or 2:1 to 12:1. Exemplary N/P ranges include about 3:1, about 6:1, about 12:1 and about 22:1.

Nanoparticle compositions can be designed for one or more specific applications or targets. For example, a nanoparticle composition can be designed to deliver a therapeutic and/or prophylactic agent such as an RNA to a particular cell, tissue, organ, or system or group thereof in a mammal's body. Physiochemical properties of nanoparticle compositions can be altered in order to increase selectivity for particular bodily targets. For instance, particle sizes can be adjusted based on the fenestration sizes of different organs. The therapeutic and/or prophylactic agent included in a nanoparticle composition can also be selected based on the desired delivery target or targets. For example, a therapeutic and/or prophylactic agent can be selected for a particular indication, condition, disease, or disorder and/or for delivery to a particular cell, tissue, organ, or system or group thereof (e.g., localized or specific delivery). In certain embodiments, a nanoparticle composition can include an mRNA encoding a polypeptide of interest capable of being translated within a cell to produce the polypeptide of interest. Such a composition can be designed to be specifically delivered to a particular organ. In certain embodiments, a composition can be designed to be specifically delivered to a mammalian liver.

The amount of a therapeutic and/or prophylactic agent in a nanoparticle composition can depend on the size, composition, desired target and/or application, or other properties of the nanoparticle composition as well as on the properties of the therapeutic and/or prophylactic agent. For example, the amount of an RNA useful in a nanoparticle composition can depend on the size, sequence, and other characteristics of the RNA. The relative amounts of a therapeutic and/or prophylactic agent and other elements (e.g., lipids) in a nanoparticle composition can also vary. In some embodiments, the wt/wt ratio of the lipid component to a therapeutic and/or prophylactic agent in a nanoparticle composition can be from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1, 20:1, 22:1, 25:1, 30:1, 35:1, 40:1, 45:1, 50:1, and 60:1. For example, the wt/wt ratio of the lipid component to a therapeutic and/or prophylactic agent can be from about 10:1 to about 40:1. In certain embodiments, the wt/wt ratio is about 20:1. The amount of a therapeutic and/or prophylactic agent in a nanoparticle composition can, for example, be measured using absorption spectroscopy (e.g., ultraviolet-visible spectroscopy).

In some embodiments, a nanoparticle composition includes one or more RNAs, and the one or more RNAs, lipids, and amounts thereof can be selected to provide a specific NP ratio. The N:P ratio of the composition refers to the molar ratio of nitrogen atoms in one or more lipids to the number of phosphate groups in an RNA. In some embodiments, a lower N:P ratio is selected. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

The physical properties of a nanoparticle composition can depend on the components thereof. For example, a nanoparticle composition including cholesterol as a structural lipid can have different characteristics compared to a nanoparticle composition that includes a different structural lipid. Similarly, the characteristics of a nanoparticle composition can depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

In various embodiments, the mean size of a nanoparticle composition can be between 10s of nm and 100s of nm. For example, the mean size can be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 un, 130 un, 135 un, 140 un, 145 urn, or 150 nm. In some embodiments, the mean size of a nanoparticle composition can be from about 50 nm to about 100 un, from about 50 un to about 90 un, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 un. In certain embodiments, the mean size of a nanoparticle composition can be from about 70 nm to about 100 nm. In some embodiments, the mean size can be about 80 un. In other embodiments, the mean size can be about 100 un. In some embodiments, the nanoparticle composition comprises a plurality of nanoparticles, and the mean size of the plurality of nanoparticles is from about 40 nm to about 150 nm. In some embodiments, the mean size of the plurality of particles is from about 50 nm to about 100 nm. In some embodiments, the mean size of the plurality of particles is about 95 nm.

A nanoparticle composition can be relatively homogenous. A polydispersity index can be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition can be from about 0.10 to about 0.20. In some embodiments, the nanoparticle composition comprises a plurality of nanoparticles, and the polydispersity index (PDI) of the nanoparticle composition is from about 0 to about 0.25. In some embodiments, the PDI of the nanoparticle composition is less than 0.1.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition can be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic agent describes the amount of therapeutic and/or prophylactic agent that is encapsulated or otherwise associated with a nanoparticle composition after preparation, relative to the initial amount provided. The encapsulation efficiency is desirably high (e.g., close to 100%). The encapsulation efficiency can be measured, for example, by comparing the amount of therapeutic and/or prophylactic agent in a solution containing the nanoparticle composition before and after breaking up the nanoparticle composition with one or more organic solvents or detergents. Fluorescence can be used to measure the amount of free therapeutic and/or prophylactic agent (e.g., RNA) in a solution. For the nanoparticle compositions described herein, the encapsulation efficiency of a therapeutic and/or prophylactic agent can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%. In some embodiments, the nanoparticle composition comprises a nucleic acid as a therapeutic agent, and the encapsulation efficiency of said nucleic acid is at least about 50%. In some embodiments, the encapsulation efficiency of said nucleic acid is at least about 80%. In some embodiments, the encapsulation efficiency of said nucleic acid is at least about 90%.

A nanoparticle composition can optionally comprise one or more coatings. For example, a nanoparticle composition can be formulated in a capsule, film, or tablet having a coating. A capsule, film, or tablet including a composition described herein can have any useful size, tensile strength, hardness, or density.

5.3.2 Cationic Lipids

In one embodiment, the cationic lipid contained in the sphingomyelin-containing compositions, nanoparticle compositions, or nanoparticles described herein is a cationic lipid described in International Patent Publication No. WO2021204175, the entirety of which is incorporated herein by reference.

In one embodiment, the cationic lipid is a compound of Formula (01-I):

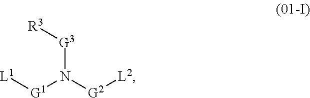

or a pharmaceutically acceptable salt, prodrug or stereoisomer thereof, wherein:
G¹ and G² are each independently a bond, C₂-C₁₂ alkylene, or C₂-C₁₂ alkenylene, wherein one or more —CH₂— in the alkylene or alkenylene is optionally replaced by —O—;
L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═R)R^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —(C₆-C₁₀ arylene)-R², -(6- to 10-membered heteroarylene)-R², or R²;
R¹ and R² are each independently C₆-C₃₂ alkyl or C₆-C₃₂ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₃₂ alkyl or C₂-C₃₂ alkenyl;
G³ is C₂-C₂₄ alkylene, C₂-C₂₄ alkenylene, C₃-C₈ cycloalkylene, or C₃-C₈ cycloalkenylene;
R³ is —N(R⁴)R⁵;
R⁴ is C₃-C₈ cycloalkyl, C₃-C₈ cycloalkenyl, 4- to 8-membered heterocyclyl, or C₆-C₁₀ aryl; or R⁴, G³ or part of G³, together with the nitrogen to which they are attached form a cyclic moiety;
R⁵ is C₁-C₁₂ alkyl or C₃-C₈ cycloalkyl; or R⁴, R⁵, together with the nitrogen to which they are attached form a cyclic moiety;
x is 0, 1 or 2; and
wherein each alkyl, alkenyl, cycloalkyl, cycloalkenyl, heterocyclyl, aryl, alkylene, alkenylene, cycloalkylene, cycloalkenylene, arylene, heteroarylene, and cyclic moiety is independently optionally substituted.

In one embodiment, the cationic lipid is a compound of Formula (01-II):

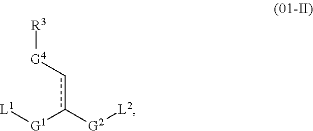

or a pharmaceutically acceptable salt, prodrug or stereoisomer thereof, wherein:
[symbol] is a single bond or a double bond;
G¹ and G² are each independently a bond, C₂-C₁₂ alkylene, or C₂-C₁₂ alkenylene, wherein one or more —CH₂— in the alkylene or alkenylene is optionally replaced by —O—;
L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —(C₆-C₁₀ arylene)-R², -(6- to 10-membered heteroarylene)-R², or R²;
R¹ and R² are each independently C₆-C₃₂ alkyl or C₆-C₃₂ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₃₂ alkyl or C₂-C₃₂ alkenyl;
G⁴ is a bond, C₁-C₂₃ alkylene, C₂-C₂₃ alkenylene, C₃-C₈ cycloalkylene, or C₃-C₈ cycloalkenylene;
R³ is —N(R⁴)R⁵;
R⁴ is C₁-C₁₂ alkyl, C₃-C₈ cycloalkyl, C₃-C₈ cycloalkenyl, 4- to 8-membered heterocyclyl, or C₆-C₁₀ aryl; or R⁴, G³ or part of G³, together with the nitrogen to which they are attached form a cyclic moiety;
R⁵ is C₁-C₁₂ alkyl or C₃-C₈ cycloalkyl, or R⁴, R⁵, together with the nitrogen to which they are attached form a cyclic moiety;
x is 0, 1 or 2; and
wherein each alkyl, alkenyl, cycloalkyl, cycloalkenyl, heterocyclyl, aryl, alkylene, alkenylene, cycloalkylene, cycloalkenylene, arylene, heteroarylene, and cyclic moiety is independently optionally substituted.

In one embodiment, the compound is a compound of Formula (01-I-B), (01-I-B′), (01-I-B″), (01-I-C), (01-I-D), or (01-I-E):

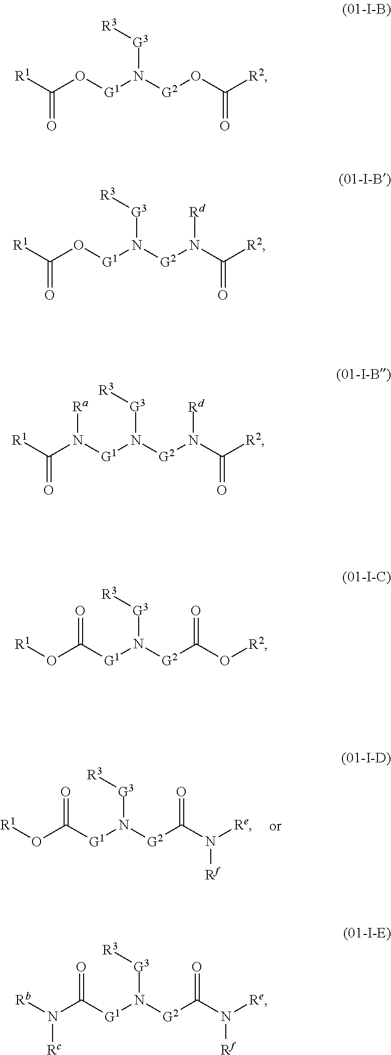

or a pharmaceutically acceptable salt, prodrug or stereoisomer thereof.

In one embodiment, G¹ and G² are each independently C₃-C₇ alkylene. In one embodiment, G¹ and G² are each independently C₅ alkylene. In one embodiment, G³ is C₂-C₄ alkylene. In one embodiment, G³ is C₂ alkylene. In one embodiment, G³ is C₄ alkylene.

In one embodiment, R³ has one of the following structures:

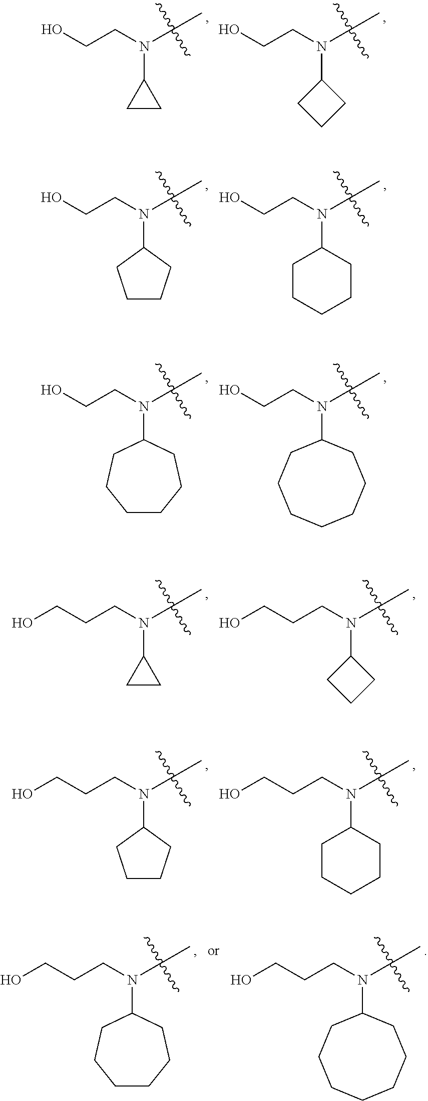

In one embodiment, R¹, R², R^{c} and R^{f} are each independently branched C₆-C₃₂ alkyl or branched C₆-C₃₂ alkenyl. In one embodiment, R¹, R², R^{c} and R^{f} are each independently branched C₆-C₂₄ alkyl or branched C₆-C₂₄ alkenyl. In one embodiment, R¹, R², R^{c} and R^{f} are each independently —R⁷—CH(R⁸)(R⁹), wherein R⁷ is C₀-C₅ alkylene, and R⁸ and R⁹ are independently C₂-C₁₀ alkyl. In one embodiment, R¹, R², R^{c} and R^{f} are each independently —R⁷—CH(R⁸)(R⁹), wherein R⁷ is C₀-C₁ alkylene, and R⁸ and R⁹ are independently C₄-C₈ alkyl.

In one embodiment, the compound is a compound in Table 1, or a pharmaceutically acceptable salt, prodrug or stereoisomer thereof.

TABLE 1

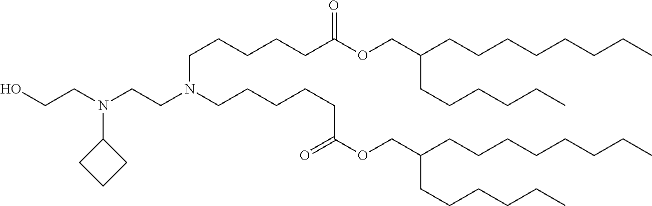 [SMILES: CCCCCCCCC(CCCCCC)COC(=O)CCCCCN(CCCCCC(=O)OCC(CCCCCC)CCCCCCCC)CCN(CCO)C1CCC1]

Compound 01-1

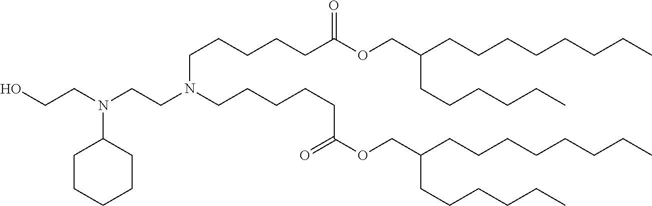 [SMILES: CCCCCCCCC(CCCCCC)COC(=O)CCCCCN(CCCCCC(=O)OCC(CCCCCC)CCCCCCCC)CCN(CCO)C1CCCCC1]

Compound 01-2

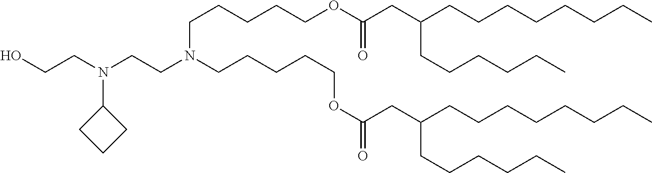 [SMILES: CCCCCCCCC(CCCCCC)CC(=O)OCCCCCN(CCCCCOC(=O)CC(CCCCCC)CCCCCCCC)CCN(CCO)C1CCC1]

Compound 01-20

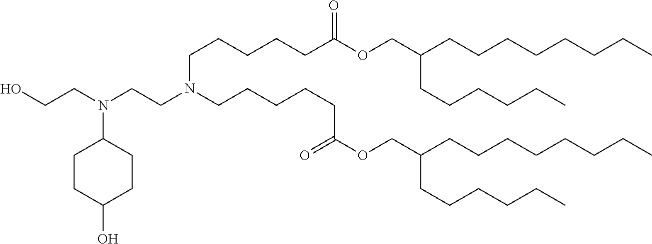 [SMILES: CCCCCCCCC(CCCCCC)COC(=O)CCCCCN(CCCCCC(=O)OCC(CCCCCC)CCCCCCCC)CCN(CCO)C1CCC(O)CC1]

Compound 01-21

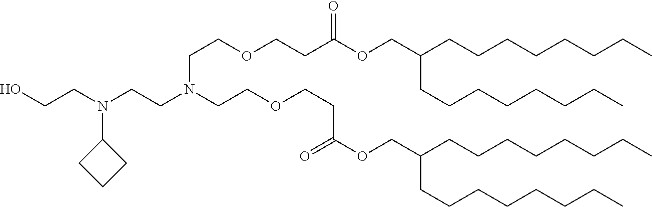 [SMILES: CCCCCCCCC(CCCCCCCC)COC(=O)CCOCCN(CCOCCC(=O)OCC(CCCCCCCC)CCCCCCCC)CCN(CCO)C1CCC1]

Compound 01-102

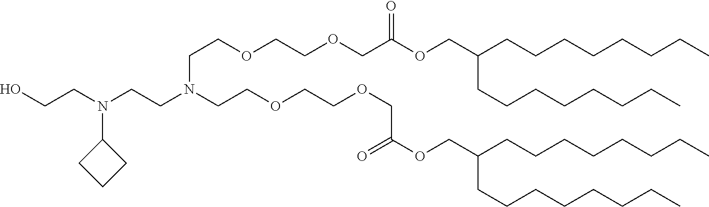 [SMILES: CCCCCCCCC(CCCCCCCC)COC(=O)COCCOCCN(CCOCCOCC(=O)OCC(CCCCCCCC)CCCCCCCC)CCN(CCO)C1CCC1]

Compound 01-103

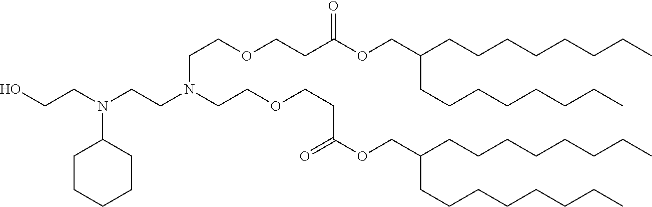 [SMILES: CCCCCCCCC(CCCCCCCC)COC(=O)CCOCCN(CCOCCC(=O)OCC(CCCCCCCC)CCCCCCCC)CCN(CCO)C1CCCCC1]

Compound 01-104

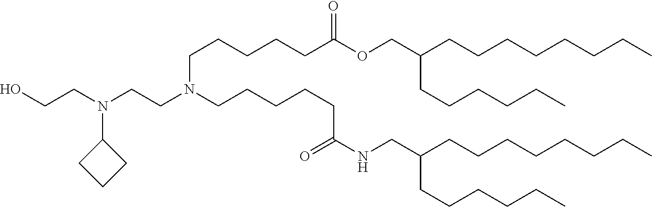 [SMILES: CCCCCCCCC(CCCCCC)CNC(=O)CCCCCN(CCCCCC(=O)OCC(CCCCCC)CCCCCCCC)CCN(CCO)C1CCC1]

Compound 01-106

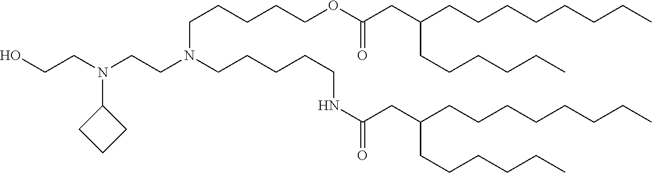 [SMILES: CCCCCCCCC(CCCCCC)CC(=O)NCCCCCN(CCCCCOC(=O)CC(CCCCCC)CCCCCCCC)CCN(CCO)C1CCC1]

Compound 01-108

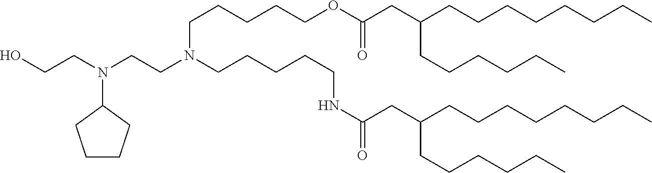 [SMILES: CCCCCCCCC(CCCCCC)CC(=O)NCCCCCN(CCCCCOC(=O)CC(CCCCCC)CCCCCCCC)CCN(CCO)C1CCCC1]

Compound 01-130

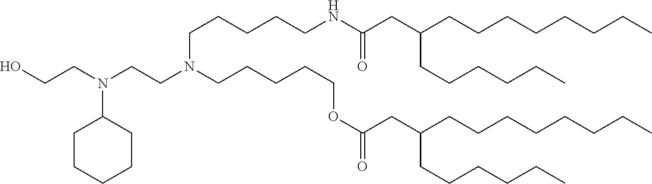 [SMILES: CCCCCCCCC(CCCCCC)CC(=O)NCCCCCN(CCCCCOC(=O)CC(CCCCCC)CCCCCCCC)CCN(CCO)C1CCCCC1]

Compound 01-131

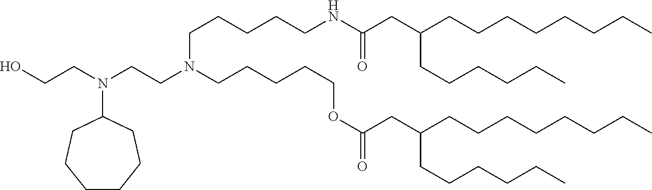 [SMILES: CCCCCCCCC(CCCCCC)CC(=O)NCCCCCN(CCCCCOC(=O)CC(CCCCCC)CCCCCCCC)CCN(CCO)C1CCCCCC1]

Compound 01-132

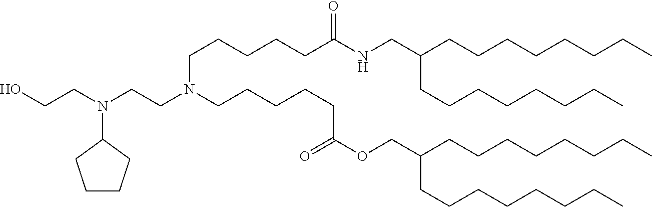 [SMILES: CCCCCCCCC(CCCCCCCC)CNC(=O)CCCCCN(CCCCCC(=O)OCC(CCCCCCCC)CCCCCCCC)CCN(CCO)C1CCCC1]

Compound 01-136

In one embodiment, the cationic lipid contained in the sphingomyelin-containing compositions, nanoparticle compositions, or nanoparticles provided herein is a cationic lipid described in International Patent Application No. PCT/CN2022/072694, the entirety of which is incorporated herein by reference. In one embodiment, the cationic lipid is a compound of Formula (02-I):

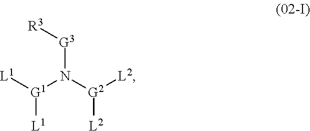

or a pharmaceutically acceptable salt, prodrug or stereoisomer thereof, wherein:
G¹ and G² are each independently C₂-C₁₂ alkylene or C₂-C₁₂ alkenylene, wherein one or more —CH₂— in G¹ and G² is optionally replaced by —O—, —C(═O)O—, or —OC(═O)—;
each L¹ is independently —OC(═O)R¹, —C(═O)OR¹, —OC(═O)OR¹, —C(═O)R¹, —OR, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c});
each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f});
R¹ and R² are each independently C₆-C₂₄ alkyl or C₆-C₂₄ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by a C₃-C₈ cycloalkylene or C₃-C₈ cycloalkenylene;
R³ is —N(R⁴)R⁵, —OR⁶, or —SR⁶;
R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
R⁵ is H, C₁-C₁₂ alkyl, C₃-C₈ cycloalkyl, C₃-C₈ cycloalkenyl, C₆-C₁₀ aryl, or 4- to 8-membered heterocycloalkyl;
R⁶ is hydrogen, C₁-C₁₂ alkyl, C₃-C₈ cycloalkyl, C₃-C₈ cycloalkenyl, or C₆-C₁₀ aryl;
x is 0, 1, or 2; and
wherein each alkyl, alkenyl, cycloalkyl, cycloalkenyl, heterocycloalkyl, aryl, alkylene, alkenylene, cycloalkylene, and cycloalkenylene is independently optionally substituted.

In one embodiment, the cationic lipid is a compound of Formula (02-II):

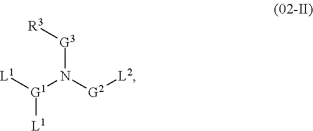

or a pharmaceutically acceptable salt, prodrug or stereoisomer thereof, wherein:
G¹ and G² are each independently C₂-C₁₂ alkylene or C₂-C₁₂ alkenylene, wherein one or more —CH₂— in G¹ and G² is optionally replaced by —O—, —C(═O)O—, or —OC(═O)—;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c});
each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f});
R¹ and R² are each independently C₆-C₂₄ alkyl or C₆-C₂₄ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by a C₃-C₈ cycloalkylene or C₃-C₈ cycloalkenylene;
R³ is —N(R⁴)R⁵, —OR⁶, or —SR⁶.
R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
R⁵ is H, C₁-C₁₂ alkyl, C₃-C₈ cycloalkyl, C₃-C₈ cycloalkenyl, C₆-C₁₀ aryl, or 4- to 8-membered heterocycloalkyl;
R⁶ is hydrogen, C₁-C₁₂ alkyl, C₃-C₈ cycloalkyl, C₃-C₈ cycloalkenyl, or C₆-C₁₀ aryl;
x is 0, 1, or 2; and
wherein each alkyl, alkenyl, cycloalkyl, cycloalkenyl, heterocycloalkyl, aryl, alkylene, alkenylene, cycloalkylene, and cycloalkenylene is independently optionally substituted.

In one embodiment, the compound is a compound of Formula (02-V-A), (02-V-B), (02-V-C), (02-V-D), (02-V-E), (02-V-F):

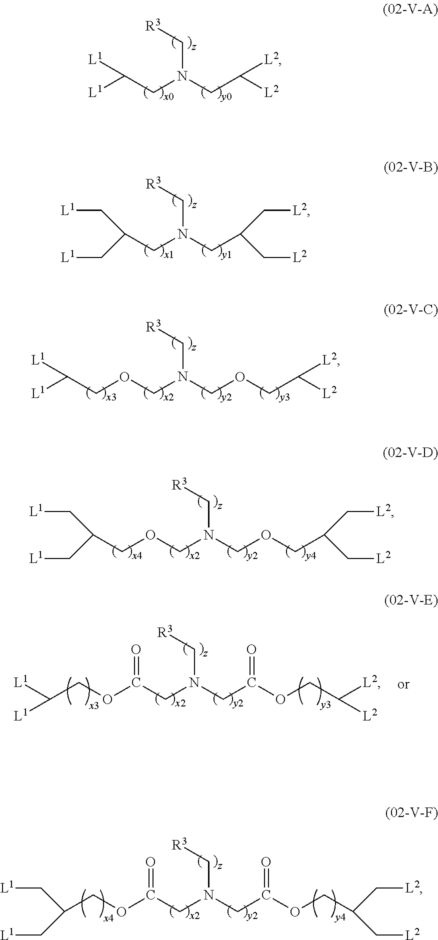

wherein z is an integer from 2 to 12,
x0 is an integer from 1 to 11;
y0 is an integer from 1 to 11;
x1 is an integer from 0 to 9;
y1 is an integer from 0 to 9;
x2 is an integer from 2 to 5;
x3 is an integer from 1 to 5;
x4 is an integer from 0 to 3;
y2 is an integer from 2 to 5;
y3 is an integer from 1 to 5; and
y4 is an integer from 0 to 3;
   or a pharmaceutically acceptable salt, prodrug or stereoisomer thereof.

In one embodiment, z is an integer from 2 to 6. In one embodiment, z is 2, 4, or 5. In one embodiment, x0 and y0 are independently 2 to 6. In one embodiment, x0 and y0 are independently 4 or 5. In one embodiment, x1 and y1 are independently 2 to 6. In one embodiment, x1 and y1 are independently 4 or 5. In one embodiment, x2 and y2 are independently an integer from 2 to 5. In one embodiment, x2 and y2 are independently 3 or 5. In one embodiment, x3 and y3 are both 1. In one embodiment, x4 and y4 are independently 0 or 1.

In one embodiment, each L¹ is independently —OR¹, —OC(═O)R¹ or —C(═O)OR¹, and each L² is independently —OR², —OC(═O)R² or —C(═O)OR². In one embodiment, R¹ and R² are independently straight C₆-C₁₀ alkyl, or —R⁷—CH(R⁸)(R⁹), wherein R⁷ is C₀-C₅ alkylene, and R⁸ and R⁹ are independently C₂-C₁₀ alkyl or C₂-C₁₀ alkenyl.

In one embodiment, the compound is a compound of formula (02-VI-A), (02-VI-B), (02-VI-C), (02-VI-D), (02-VI-E), or (02-VI-F):

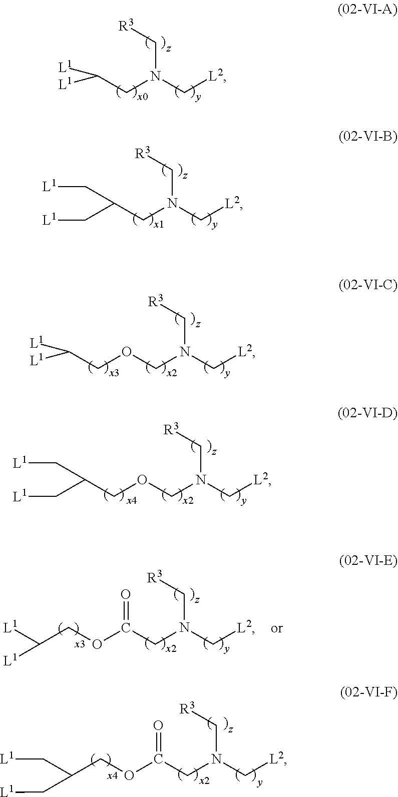

wherein z is an integer from 2 to 12;
y is an integer from 2 to 12;
x0 is an integer from 1 to 11;
x1 is an integer from 0 to 9;
x2 is an integer from 2 to 5;
x3 is an integer from 1 to 5; and
x4 is an integer from 0 to 3;
   or a pharmaceutically acceptable salt, prodrug or stereoisomer thereof.

In one embodiment, z is an integer from 2 to 6. In one embodiment, z is 2, 4 or 5. In one embodiment, x0 is 4 or 5. In one embodiment, x1 is 4 or 5. In one embodiment, x2 is an integer from 2 to 5. In one embodiment, x2 is 3 or 5. In one embodiment, x3 is 0 or 1. In one embodiment, y is an integer from 2 to 6. In one embodiment, y is 5.

In one embodiment, each L is independently —OR¹, —OC(═O)Rt or —C(═O)OR¹, and L² is —OC(═O)R² or —C(═O)OR², —NR^{d}C(═O)R², or —C(═O)NR^{e}R^{f}. In one embodiment, R¹ is straight C₆-C₁₀ alkyl or —R⁷—CH(R⁸)(R⁹), wherein R⁷ is C₀-C₅ alkylene, and R⁸ and R⁹ are independently C₂-C₁₀ alkyl or C₂-C₁₀ alkenyl. In one embodiment, R² and R^{f} are each independently straight C₆-C₁₈ alkyl, C₆-C₁₈ alkenyl, or —R⁷—CH(R⁸)(R⁹), wherein R⁷ is C₀-C₅ alkylene, and R⁸ and R⁹ are independently C₂-C₁₀ alkyl or C₂-C₁₀ alkenyl. In one embodiment, R^{d} and R^{e} are each independently H.

In one embodiment, the compound is a compound in Table 2, or a pharmaceutically acceptable salt, prodrug or stereoisomer thereof.

TABLE 2

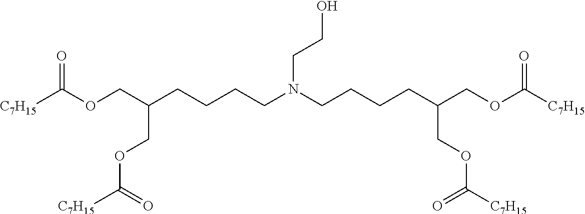

Compound 02-1

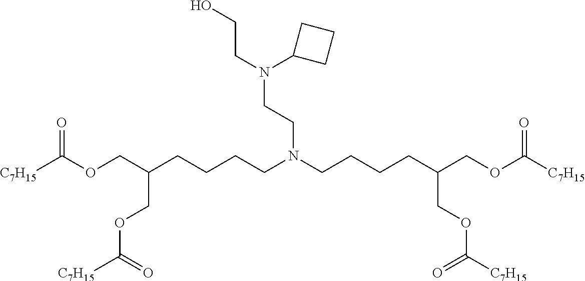

Compound 02-2

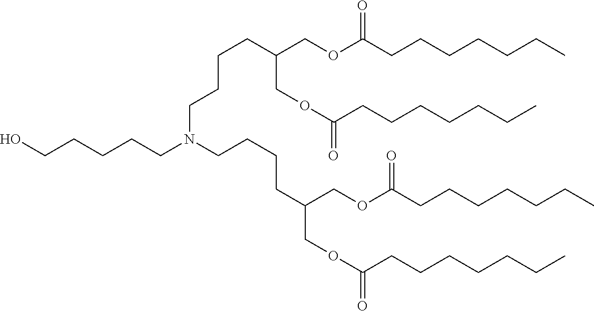 [SMILES: CCCCCCCC(=O)OCC(CCCCN(CCCCCO)CCCCC(COC(=O)CCCCCCC)COC(=O)CCCCCCC)COC(=O)CCCCCCC]

Compound 02-3

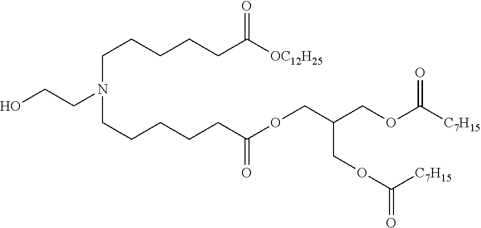

Compound 02-4

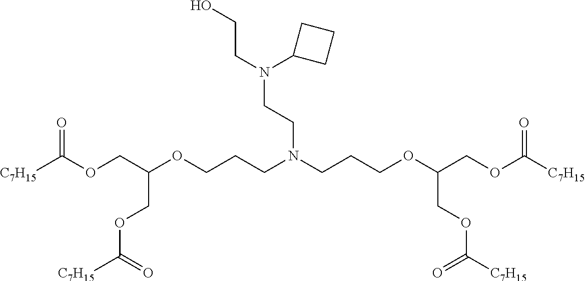

Compound 02-9

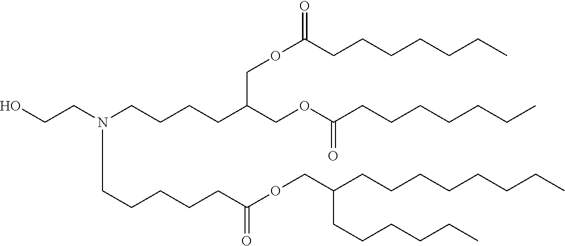 [SMILES: CCCCCCCCC(CCCCCC)COC(=O)CCCCCN(CCO)CCCCC(COC(=O)CCCCCCC)COC(=O)CCCCCCC]

Compound 02-10

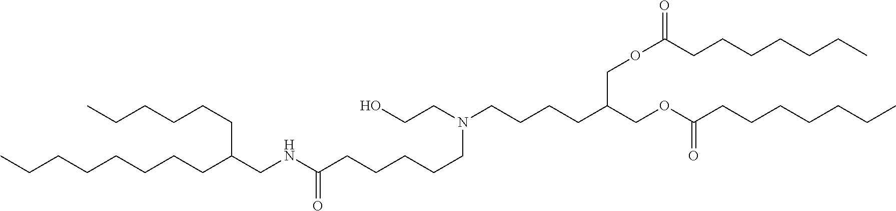 [SMILES: CCCCCCCCC(CCCCCC)CNC(=O)CCCCCN(CCO)CCCCC(COC(=O)CCCCCCC)COC(=O)CCCCCCC]

Compound 02-11

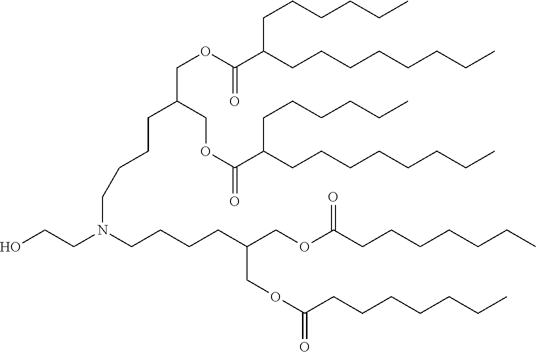 [SMILES: CCCCCCCCC(CCCCCC)C(=O)OCC(CCCCN(CCO)CCCCC(COC(=O)CCCCCCC)COC(=O)CCCCCCC)COC(=O)C(CCCCCC)CCCCCCCC]

Compound 02-12

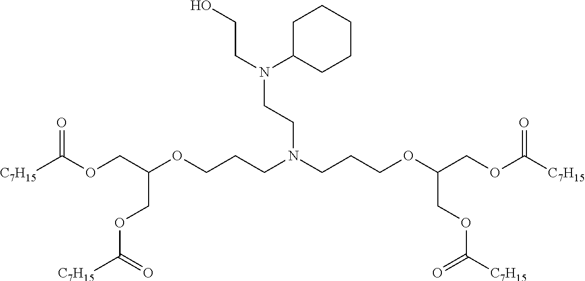

Compound 02-14

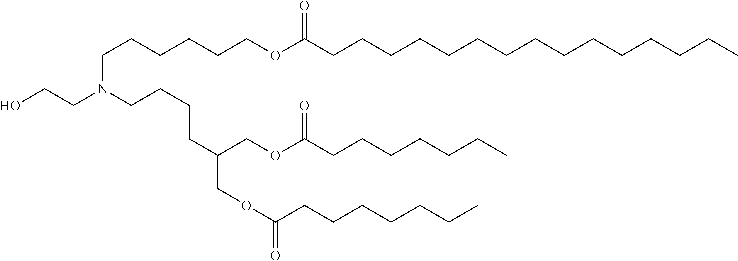 [SMILES: CCCCCCCCCCCCCCCC(=O)OCCCCCCN(CCO)CCCCC(COC(=O)CCCCCCC)COC(=O)CCCCCCC]

Compound 02-20

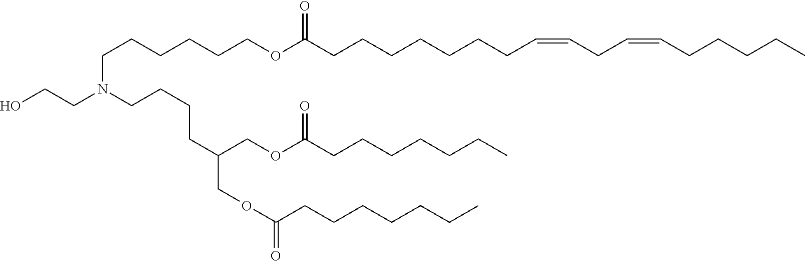 [SMILES: CCCCC/C=C\C/C=C\CCCCCCCC(=O)OCCCCCCN(CCO)CCCCC(COC(=O)CCCCCCC)COC(=O)CCCCCCC]

Compound 02-22

In one embodiment, the cationic lipid contained in the sphingomyelin-containing compositions, nanoparticle compositions, or nanoparticles described herein is a cationic lipid described in International Patent Publication No. WO2022152109, the entirety of which is incorporated herein by reference.

In one embodiment, the cationic lipid is a compound of Formula (03-I):

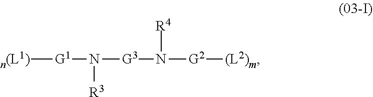

or a pharmaceutically acceptable salt, prodrug or stereoisomer thereof, wherein:
G¹ and G² are each independently a bond, C₂-C₁₂ alkylene, or C₂-C₁₂ alkenylene, wherein one or more —CH₂— in G¹ and G² is optionally replaced by —O—;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c}), —(C₆-C₁₀ arylene)-R¹, -(6- to 10-membered heteroarylene)-R¹, -(4- to 8-membered heterocyclylene)-R¹, or R¹;
each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f}), —(C₆-C₁₀ arylene)-R², -(6- to 10-membered heteroarylene)-R², -(4- to 8-membered heterocyclylene)-R², or R²;
R¹ and R² are each independently C₆-C₂₄ alkyl or C₆-C₂₄ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety; or R³, G³ or part of G³, together with the nitrogen to which they are attached form a cyclic moiety;
R⁴ is C₁-C₁₂ alkyl or C₃-C₈ cycloalkyl;
x is 0, 1, or 2;
n is 1 or 2;
m is 1 or 2; and
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

In one embodiment, the compound is a compound of Formula (03-II-A):

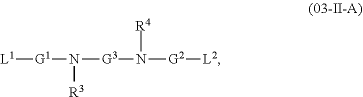

or a pharmaceutically acceptable salt, prodrug or stereoisomer thereof.

In one embodiment, the compound is a compound of Formula (03-II-B):

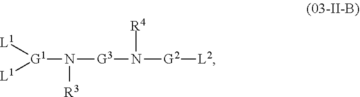

or a pharmaceutically acceptable salt, prodrug or stereoisomer thereof.

In one embodiment, the compound is a compound of Formula (03-II-C):

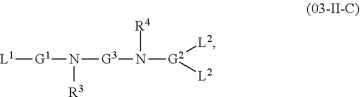

or a pharmaceutically acceptable salt, prodrug or stereoisomer thereof.

In one embodiment, the compound is a compound of Formula (03-II-D):

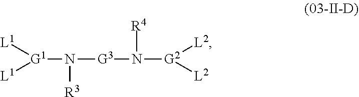

or a pharmaceutically acceptable salt, prodrug or stereoisomer thereof.

In one embodiment, G¹ and G² are each independently C₂-C₁₂ alkylene. In one embodiment, G¹ and G² are each independently C₅ alkylene. In one embodiment, G³ is C₂-C₆ alkylene.

In one embodiment, R³ is C₁-C₁₂ alkyl, C₂-C₁₂ alkenyl, or C₃-C₈ cycloalkyl. In one embodiment, R³ is C₃-C₈ cycloalkyl. In one embodiment, R³ is unsubstituted. In one embodiment, R⁴ is substituted C₁-C₁₂ alkyl. In one embodiment, R⁴ is —CH₂CH₂OH.

In one embodiment, L¹ is —OC(═O)R¹, —C(═O)OR¹, —NR^{a}C(═O)R¹, or —C(═O)NR^{b}R^{c}; and L² is —OC(═O)R², —C(═O)OR², —NR^{d}C(═O)R², or —C(═O)NR^{e}R^{f}. In one embodiment, R¹, R², R^{c}, and R^{f} are each independently straight C₆-C₁₅ alkyl, straight C₆-C₁₈ alkenyl, or —R⁷—CH(R⁸)(R⁹), wherein R⁷ is C₀-C₅ alkylene, and R⁸ and R⁹ are independently C₂-C₁₀ alkyl or C₂-C₁₀ alkenyl. In one embodiment, R¹, R², R^{c}, and R^{f} are each independently straight C₇-C₁₅ alkyl, straight C₇-C₁₅ alkenyl, or —R⁷—CH(R⁸)(R⁹), wherein R⁷ is C₀-C₁ alkylene, and R⁸ and R⁹ are independently C₄-C₈ alkyl or C₆-C₁₀ alkenyl. In one embodiment, R^{a}, R^{b}, R^{d}, and R^{e} are each independently H.

In one embodiment, the compound is a compound in Table 3, or a pharmaceutically acceptable salt, prodrug or stereoisomer thereof.

TABLE 3

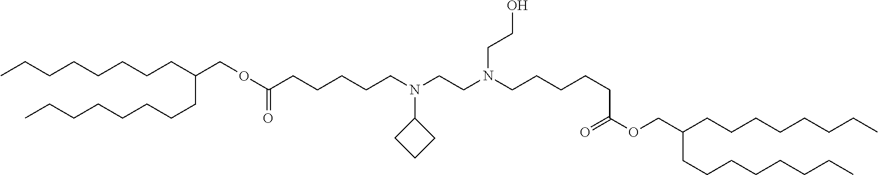 [SMILES: CCCCCCCCC(CCCCCCCC)COC(=O)CCCCCN(CCO)CCN(CCCCCC(=O)OCC(CCCCCCCC)CCCCCCCC)C1CCC1]

Compound 03-10

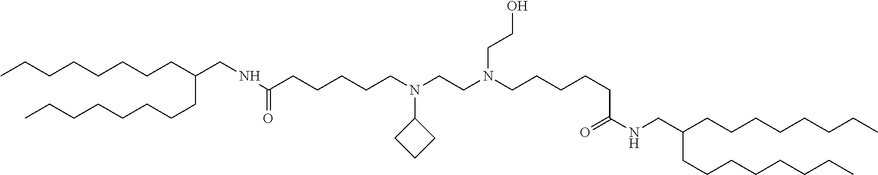 [SMILES: CCCCCCCCC(CCCCCCCC)CNC(=O)CCCCCN(CCO)CCN(CCCCCC(=O)NCC(CCCCCCCC)CCCCCCCC)C1CCC1]

Compound 03-11

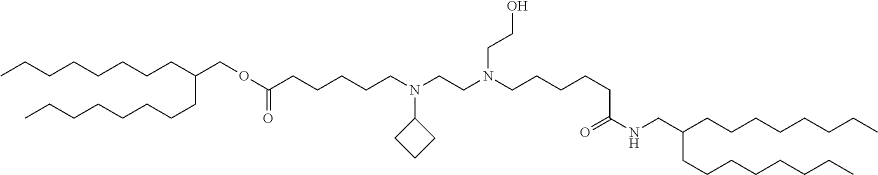 [SMILES: CCCCCCCCC(CCCCCCCC)CNC(=O)CCCCCN(CCO)CCN(CCCCCC(=O)OCC(CCCCCCCC)CCCCCCCC)C1CCC1]

Compound 03-15

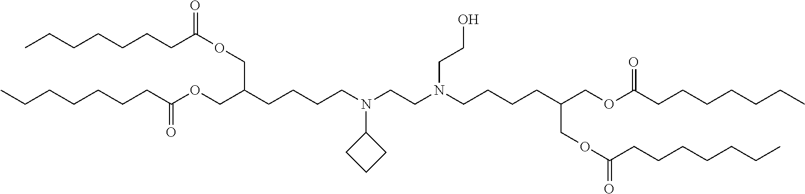 [SMILES: CCCCCCCC(=O)OCC(CCCCN(CCO)CCN(CCCCC(COC(=O)CCCCCCC)COC(=O)CCCCCCC)C1CCC1)COC(=O)CCCCCCC]

Compound 03-71

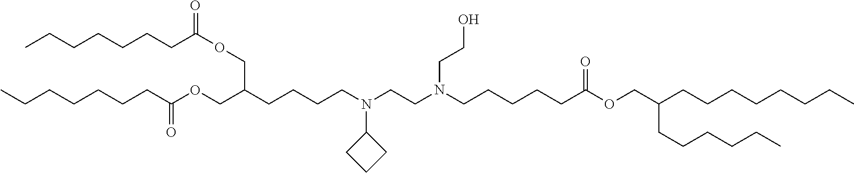 [SMILES: CCCCCCCCC(CCCCCC)COC(=O)CCCCCN(CCO)CCN(CCCCC(COC(=O)CCCCCCC)COC(=O)CCCCCCC)C1CCC1]

Compound 03-135

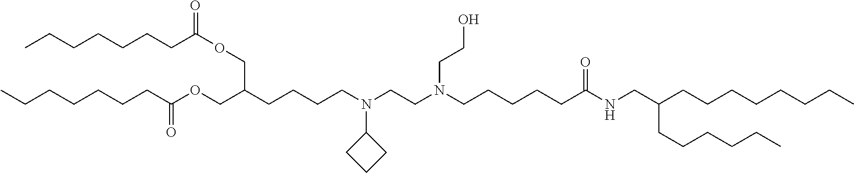 [SMILES: CCCCCCCCC(CCCCCC)CNC(=O)CCCCCN(CCO)CCN(CCCCC(COC(=O)CCCCCCC)COC(=O)CCCCCCC)C1CCC1]

Compound 03-136

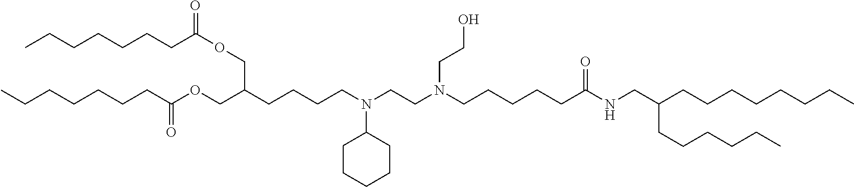 [SMILES: CCCCCCCCC(CCCCCC)CNC(=O)CCCCCN(CCO)CCN(CCCCC(COC(=O)CCCCCCC)COC(=O)CCCCCCC)C1CCCCC1]

Compound 03-140

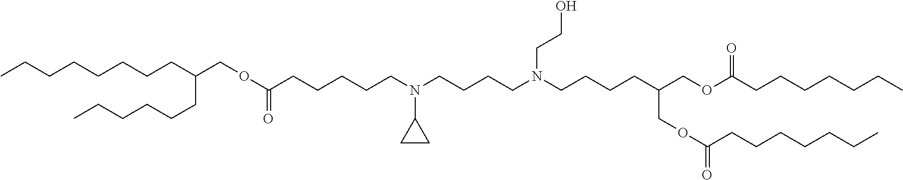 [SMILES: CCCCCCCCC(CCCCCC)COC(=O)CCCCCN(CCCCN(CCO)CCCCC(COC(=O)CCCCCCC)COC(=O)CCCCCCC)C1CC1]

Compound 03-190

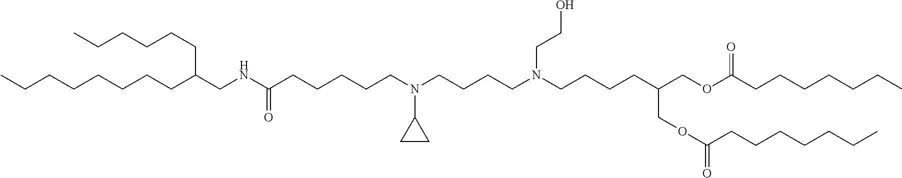 [SMILES: CCCCCCCCC(CCCCCC)CNC(=O)CCCCCN(CCCCN(CCO)CCCCC(COC(=O)CCCCCCC)COC(=O)CCCCCCC)C1CC1]

Compound 03-195

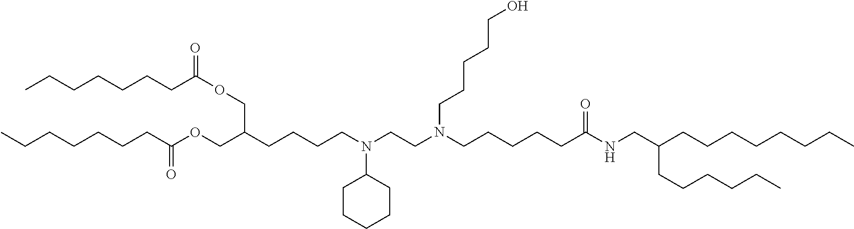 [SMILES: CCCCCCCCC(CCCCCC)CNC(=O)CCCCCN(CCCCCO)CCN(CCCCC(COC(=O)CCCCCCC)COC(=O)CCCCCCC)C1CCCCC1]

Compound 03-209

In one embodiment, the cationic lipid contained in the particles or compositions provided herein is a cationic lipid described in International Patent Application No. PCT/CN2022/094227, the entirety of which is incorporated herein by reference.

In one embodiment, the cationic lipid is a compound of Formula (04-1):

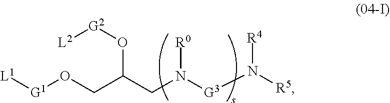

or a pharmaceutically acceptable salt, prodrug or stereoisomer thereof, wherein:
G¹ and G² are each independently a bond, C₂-C₁₂ alkylene, or C₂-C₁₂ alkenylene;
L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —(C₆-C₁₀ arylene)-R², -(6- to 10-membered heteroarylene)-R², or R²;
R¹ and R² are each independently C₅-C₃₂ alkyl or C₅-C₃₂ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₃₂ alkyl or C₂-C₃₂ alkenyl;
R⁰ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
G³ is C₂-C₁₂ alkylene or C₂-C₁₂ alkenylene;
R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
R⁵ is C₁-C₁₂ alkyl, C₃-C₈ cycloalkyl, C₃-C₈ cycloalkenyl, C₆-C₁₀ aryl, or 4- to 8-membered heterocycloalkyl;
x is 0, 1, or 2;
s is 0 or 1; and
wherein each alkyl, alkenyl, cycloalkyl, cycloalkenyl, heterocycloalkyl, aryl, alkylene, alkenylene, arylene, and heteroarylene, is independently optionally substituted.

In one embodiment, the cationic lipid is a compound of Formula (04-III):

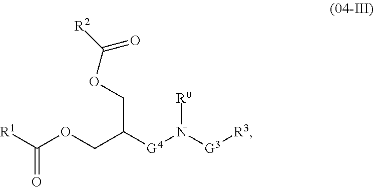

or a pharmaceutically acceptable salt, prodrug or stereoisomer thereof, wherein:
R¹ and R² are each independently C₅-C₃₂ alkyl or C₅-C₃₂ alkenyl;
R⁰ is C₁-C₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
G³ is C₂-C₁₂ alkylene or C₂-C₁₂ alkenylene;
G⁴ is C₂-C₁₂ alkylene or C₂-C₁₂ alkenylene;
R³ is —N(R⁴)R⁵ or —OR⁶;
R⁴ is C₁-C₁₂ alkyl, C₂-C₁₂ alkenyl, C₃-C₈ cycloalkyl, C₃-C₈ cycloalkenyl, C₆-C₁₀ aryl, or 4- to 8-membered heterocycloalkyl;
R⁵ is C₁-C₁₂ alkyl, C₃-C₈ cycloalkyl, C₃-C₈ cycloalkenyl, C₆-C₁₀ aryl, or 4- to 8-membered heterocycloalkyl; or R⁴, R⁵, together with the nitrogen to which they are attached form a cyclic moiety;
R⁶ is hydrogen, C₁-C₁₂ alkyl, C₃-C₈ cycloalkyl, C₃-C₈ cycloalkenyl, or C₆-C₁₀ aryl; and
wherein each alkyl, alkenyl, cycloalkyl, cycloalkenyl, heterocycloalkyl, aryl, alkylene, alkenylene, and cyclic moiety is independently optionally substituted.

In one embodiment, the compound is a compound of Formula (04-IV):

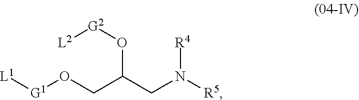

or a pharmaceutically acceptable salt, prodrug or stereoisomer thereof.

In one embodiment, G³ is C₂-C₄ alkylene. In one embodiment, G⁴ is C₂-C₄ alkylene.

In one embodiment, R⁰ is C₁-C₆ alkyl. In one embodiment, R³ is —OH. In one embodiment, R³ is —N(R⁴)R⁵. In one embodiment, R⁴ is C₃-C₈ cycloalkyl. In one embodiment, R⁴ is unsubstituted. In one embodiment, R⁵ is —CH₂CH₂OH.

In one embodiment, L¹ is —OC(═O)R¹, —C(═O)OR¹, —C(═O)R¹, —C(═O)NR^{b}R^{c}, or R¹; and L² is —OC(═O)R², —C(═O)OR², —C(═O)R², —C(═O)NR^{e}R^{f}, or R². In one embodiment, R¹ and R² are each independently branched C₆-C₂₄ alkyl or branched C₆-C₂₄ alkenyl. In one embodiment, R¹ and R² are each independently —R⁷—CH(R⁸)(R⁹), wherein R⁷ is C₁-C₅ alkylene, and R⁸ and R⁹ are independently C₂-C₁₀ alkyl or C₂-C₁₀ alkenyl. In one embodiment, R¹ is straight C₆-C₂₄ alkyl and R² is branched C₆-C₂₄ alkyl. In one embodiment, R¹ is straight C₆-C₂₄ alkyl and R² is —R⁷—CH(R⁸)(R⁹), wherein R⁷ is C₁-C₅ alkylene, and R⁸ and R⁹ are independently C₂-C₁₀ alkyl.

In one embodiment, the compound is a compound in Table 4, or a pharmaceutically acceptable salt, prodrug or stereoisomer thereof.

TABLE 4

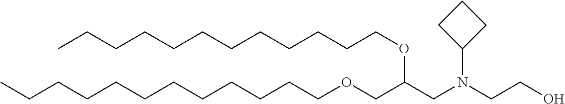 [SMILES: CCCCCCCCCCCCOCC(CN(CCO)C1CCC1)OCCCCCCCCCCCC]

Compound 04-1

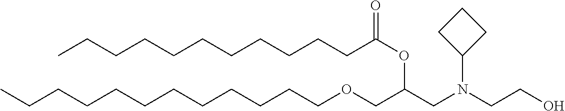 [SMILES: CCCCCCCCCCCCOCC(CN(CCO)C1CCC1)OC(=O)CCCCCCCCCCC]

Compound 04-2

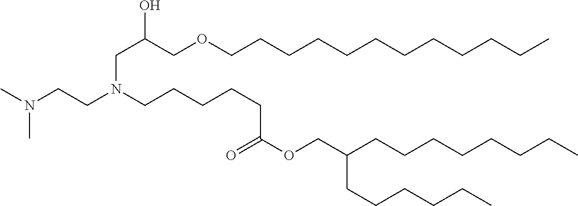 [SMILES: CCCCCCCCCCCCOCC(O)CN(CCCCCC(=O)OCC(CCCCCC)CCCCCCCC)CCN(C)C]

Compound 04-7

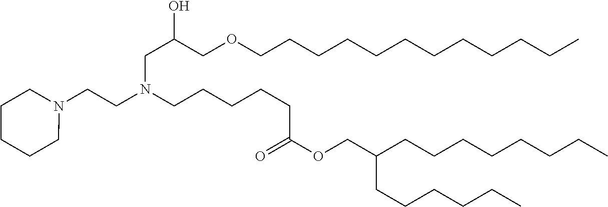 [SMILES: CCCCCCCCCCCCOCC(O)CN(CCCCCC(=O)OCC(CCCCCC)CCCCCCCC)CCN1CCCCC1]

Compound 04-8

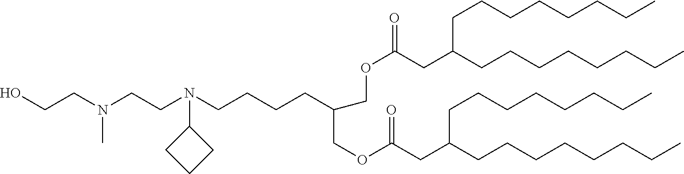 [SMILES: CCCCCCCCC(CCCCCCCC)CC(=O)OCC(CCCCN(CCN(C)CCO)C1CCC1)COC(=O)CC(CCCCCCCC)CCCCCCCC]

Compound 04-65

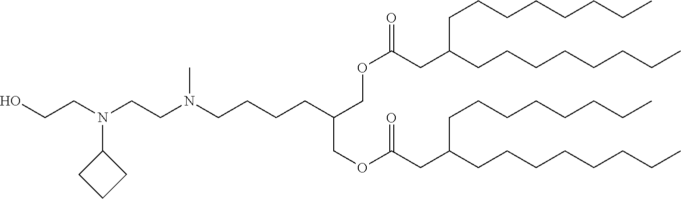 [SMILES: CCCCCCCCC(CCCCCCCC)CC(=O)OCC(CCCCN(C)CCN(CCO)C1CCC1)COC(=O)CC(CCCCCCCC)CCCCCCCC]

Compound 04-66

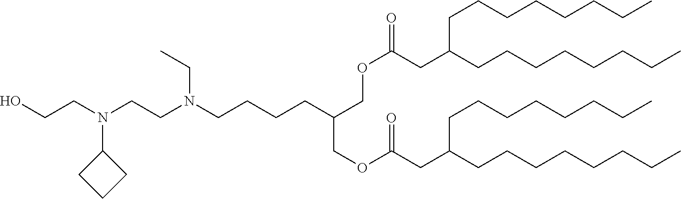 [SMILES: CCCCCCCCC(CCCCCCCC)CC(=O)OCC(CCCCN(CC)CCN(CCO)C1CCC1)COC(=O)CC(CCCCCCCC)CCCCCCCC]

Compound 04-67

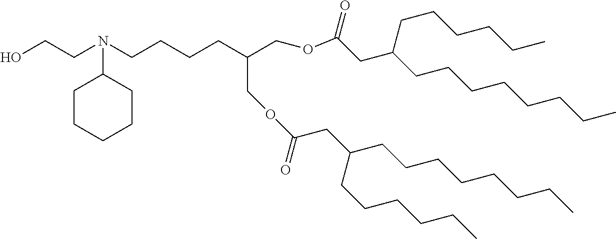 [SMILES: CCCCCCCCC(CCCCCC)CC(=O)OCC(CCCCN(CCO)C1CCCCC1)COC(=O)CC(CCCCCC)CCCCCCCC]

Compound 04-68

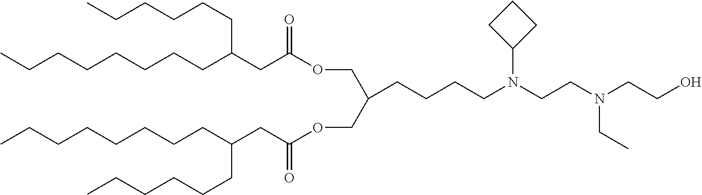 [SMILES: CCCCCCCCC(CCCCCC)CC(=O)OCC(CCCCN(CCN(CC)CCO)C1CCC1)COC(=O)CC(CCCCCC)CCCCCCCC]

Compound 04-69

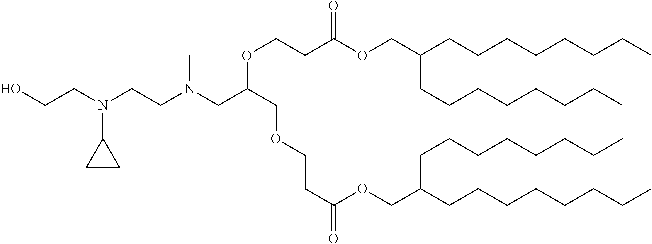 [SMILES: CCCCCCCCC(CCCCCCCC)COC(=O)CCOCC(CN(C)CCN(CCO)C1CC1)OCCC(=O)OCC(CCCCCCCC)CCCCCCCC]

Compound 04-70

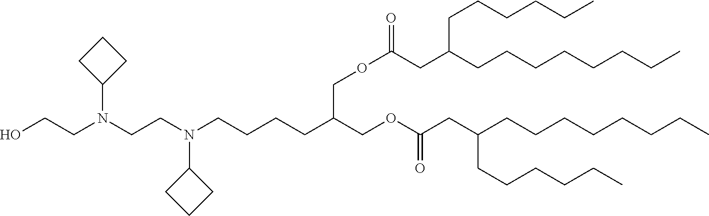 [SMILES: CCCCCCCCC(CCCCCC)CC(=O)OCC(CCCCN(CCN(CCO)C1CCC1)C1CCC1)COC(=O)CC(CCCCCC)CCCCCCCC]

Compound 04-79

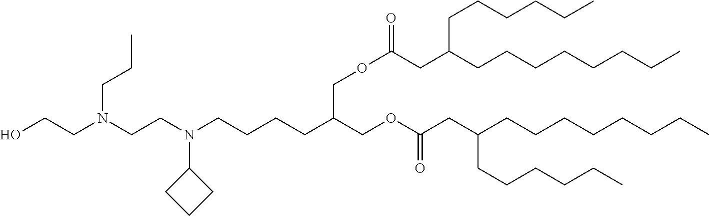 [SMILES: CCCCCCCCC(CCCCCC)CC(=O)OCC(CCCCN(CCN(CCC)CCO)C1CCC1)COC(=O)CC(CCCCCC)CCCCCCCC]

Compound 04-80

In one embodiment, the cationic lipid contained in the particles or compositions provided herein is a cationic lipid described in U.S. Pat. Nos. 10,442,756B2, 9,868,691B2, and 9,8686,921B2, the entire teachings of which are incorporated herein by reference.

In one embodiment, the cationic lipid is a compound Formula (05-I):

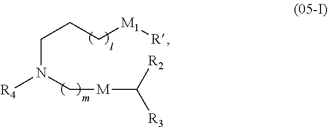

or a salt or isomer thereof, wherein
l is selected from 1, 2, 3, 4, and 5;
m is selected from 5, 6, 7, 8, and 9;
M₁ is a bond or M′;
R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, or heteroaryl, and each n is selected from 1, 2, 3, 4, or 5;
M and M′ are independently selected from —C(O)O—, —OC(O)—, —C(O)N(R′)—, —P(O)(OR′)O—, —S—S—, an aryl group, and a heteroaryl group; and
R₂ and R₃ are both C₁₋₁₄ alkyl, or C₂₋₁₄ alkenyl,
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H; and
R′ is a linear alkyl.

In one embodiment, the compound is SM102 or Lipid 5:

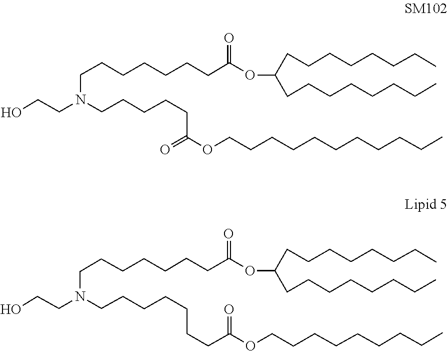 [SMILES: CCCCCCCCCCCOC(=O)CCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCCCC)CCCCCCCC]

In one embodiment, the cationic lipid contained in the particles or compositions provided herein is a cationic lipid described in U.S. Pat. No. 10,166,298B2, the entire teachings of which are incorporated herein by reference.

In one embodiment, the cationic lipid is a compound of Formula (06-I):

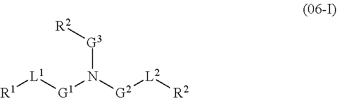

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In one embodiment, the compound is a compound in Table 5, or a pharmaceutically acceptable salt, prodrug or stereoisomer thereof.

TABLE 5

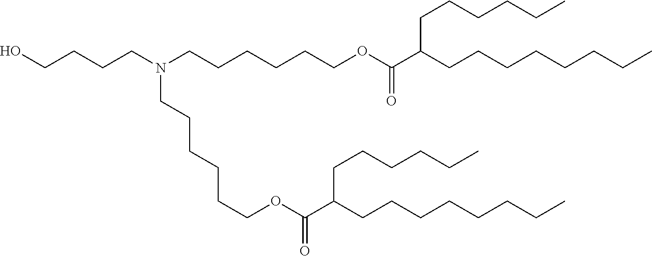 [SMILES: CCCCCCCCC(CCCCCC)C(=O)OCCCCCCN(CCCCO)CCCCCCOC(=O)C(CCCCCC)CCCCCCCC]

Compound 06-1

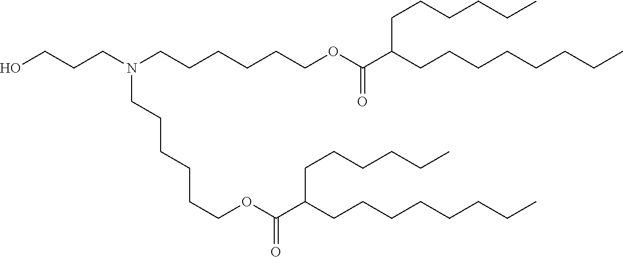 [SMILES: CCCCCCCCC(CCCCCC)C(=O)OCCCCCCN(CCCO)CCCCCCOC(=O)C(CCCCCC)CCCCCCCC]

Compound 06-2

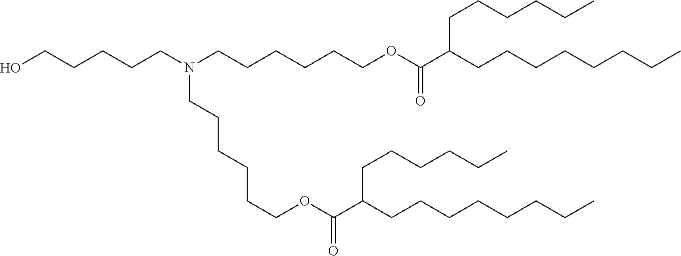 [SMILES: CCCCCCCCC(CCCCCC)C(=O)OCCCCCCN(CCCCCO)CCCCCCOC(=O)C(CCCCCC)CCCCCCCC]

Compound 06-3

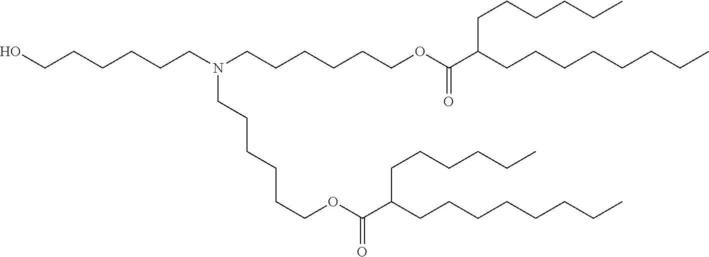 [SMILES: CCCCCCCCC(CCCCCC)C(=O)OCCCCCCN(CCCCCCO)CCCCCCOC(=O)C(CCCCCC)CCCCCCCC]

Compound 06-4

It is understood that any embodiment of the compounds provided herein, as set ort above, and any specific substituent and/or variable in the compound provided herein, as set forth above, may be independently combined with other embodiments and/or substituents and/or variables of the compounds to form embodiments not specifically set forth above. In addition, in the event that a list of substituents and/or variables is listed for any particular group or variable, it is understood that each individual substituent and/or variable may be deleted from the particular embodiment and/or claim and that the remaining list of substituents and/or variables will be considered to be within the scope of embodiments provided herein.

It is understood that in the present description, combinations of substituents and/or variables of the depicted formulae are permissible only if such contributions result in stable compounds.

### 5.3.3 Other Ionizable Lipids

In one embodiment, the sphingomyelin-containing composition, nanoparticle compositions, or nanoparticles provided herein comprises one or more charged or ionizable lipids. These charged or ionizable lipids can either replace the cationic lipids described herein or be included in addition to the cationic lipids described herein. Without being bound by the theory, it is contemplated that certain charged or zwitterionic lipid components of a nanoparticle composition resembles the lipid component in the cell membrane, thereby can improve cellular uptake of the nanoparticle. Exemplary charged or ionizable lipids that can form part of the present nanoparticle composition include but are not limited to 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLinDMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3 [(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)- -octadeca-9,12-dien-1-yloxy]propan-1-amine (Octyl-CLinDMA (2R)), (2S)-2-({8-[(3β)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), (12Z,15Z)-N,N-dimethyl-2-nonylhenicosa-12,15-den-1-amine, N,N-dimethyl-1-{(1S,2R)-2-octylcyclopropyl}heptadecan-8-amine. Additional exemplary charged or ionizable lipids that can form part of the present nanoparticle composition include the lipids (e.g., lipid 5) described in Sabnis et al. “A Novel Amino Lipid Series for mRNA Delivery: Improved Endosomal Escape and Sustained Pharmacology and Safety in Non-human Primates”, Molecular Therapy Vol. 26 No 6, 2018, the entirety of which is incorporated herein by reference. Additional exemplary charged or ionizable lipids that can form part of the present nanoparticle composition include lipids described in any of WO2010053572A9, WO2013016058A1, WO2013086373A, WO2013149140A1, WO2015184256A2, WO2015199952A1, WO2017180917A2, WO2017049245, WO2018107026A1, WO2019036008A1, WO2020061367A1, WO2020146805A1, WO2020072324A1, WO2020002525A1, U.S. Pat. No. 8,722,082B2, U.S. Pat. Nos. 9,687,550, 10,077,232B2, 10,059,655, 10,639,279B2, US20160317458A1, US20160376224A1, US20160151284A1, US20160244761A1, US20180169268A1, US2019151461A1, US20200308111A1, US20200308111A1, and US20200331841A1, the content of each of which is enclosed herein by reference in its entirety.

In some embodiments, suitable cationic lipids include N-[1-(2,3-dioleyloxy)propyl]-N,N,N-trimethylammonium chloride (DOTMA); N-[1-(2,3-dioleoyloxy)propyl]-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1); N1-[2-((1S)-1-[(3-aminopropyl)amino]-4-[di(3-amino-propyl)amino]butylcarboxamido)ethyl]-3,4-di[oleyloxy]-benzamide (MVL5); dioctadecylamido-glycylspermine (DOGS); 3b-[N—(N′,N′-dimethylaminoethyl)carbamoyl]cholesterol (DC-Chol); dioctadecyldimethylammonium bromide (DDAB); SAINT-2, N-methyl-4-(dioleyl)methylpyridinium; 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); di-alkylated amino acid (DILA²) (e.g., C18:1-norArg-C16); dioleyldimethylammonium chloride (DODAC); 1-palmitoyl-2-oleoyl-sn-glycero-3-ethylphosphocholine (POEPC); 1,2-dimyristoleoyl-sn-glycero-3-ethylphosphocholine (MOEPC); (R)-5-(dimethylamino)pentane-1,2-diyl dioleate hydrochloride (DODAPen-Cl); (R)-5-guanidinopentane-1,2-diyl dioleate hydrochloride (DOPen-G); and (R)-N,N,N-trimethyl-4,5-bis(oleoyloxy)pentan-1-aminium chloride (DOTAPen). Also suitable are cationic lipids with headgroups that are charged at physiological pH, such as primary amines (e.g., DODAG N′,N′-dioctadecyl-N-4,8-diaza-10-aminodecanoylglycine amide) and guanidinium head groups (e.g., bis-guanidinium-spermidine-cholesterol (BGSC), bis-guanidiniumtren-cholesterol (BGTC), PONA, and (R)-5-guanidinopentane-1,2-diyl dioleate hydrochloride (DOPen-G)). Yet another suitable cationic lipid is (R)-5-(dimethylamino)pentane-1,2-diyl dioleate hydrochloride (DODAPen-Cl). In certain embodiments, the cationic lipid is a particular enantiomer or the racemic form, and includes the various salt forms of a cationic lipid as above (e.g., chloride or sulfate). For example, in some embodiments, the cationic lipid is N-[1-(2,3-dioleoyloxy)propyl]-N,N,N-trimethylammonium chloride (DOTAP-Cl) or N-[1-(2,3-dioleoyloxy)propyl]-N,N,N-trimethylammonium sulfate (DOTAP-Sulfate). In some embodiments, the cationic lipid is an ionizable cationic lipid such as, e.g., dioctadecyldimethylammonium bromide (DDAB); 1,2-dilinoleyloxy-3-dimethylaminopropane (DLinDMA); 2,2-dilinoleyl-4-(2dimethylaminoethyl)-[1,3]-dioxolane (DLin-KC2-DMA); heptatriaconta-6,9,28,31-tetraen-19-yl 4-(dimethylamino)butanoate (DLin-MC3-DMA); 1,2-dioleoyloxy-3-dimethylaminopropane (DODAP); 1,2-dioleyloxy-3-dimethylaminopropane (DODMA); and morpholinocholesterol (Mo-CHOL). In certain embodiments, a lipid nanoparticle includes a combination or two or more cationic lipids (e.g., two or more cationic lipids as above).

Additionally, in some embodiments, the charged or ionizable lipid that can form part of the present nanoparticle composition is a lipid including a cyclic amine group. Additional cationic lipids that are suitable for the formulations and methods disclosed herein include those described in WO2015199952, WO2016176330, and WO2015011633, the entire contents of each of which are hereby incorporated by reference in their entireties. Additionally, in some embodiments, the charged or ionizable lipid that can form part of the present nanoparticle composition is a lipid including a cyclic amine group. Additional cationic lipids that are suitable for the formulations and methods disclosed herein include those described in WO2015199952, WO2016176330, and WO2015011633, the entire contents of each of which are hereby incorporated by reference in their entireties.

### 5.3.4 Polymer Conjugated Lipids

In some embodiments, the lipid component of a sphingomyelin-containing composition, nanoparticle compositions, or nanoparticles provided herein can include one or more polymer conjugated lipids, such as PEGylated lipids (PEG lipids). Without being bound by the theory, it is contemplated that a polymer conjugated lipid component in a nanoparticle composition can improve of colloidal stability and/or reduce protein absorption of the nanoparticles. Exemplary cationic lipids that can be used in connection with the present disclosure include but are not limited to PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DSPE, Ceramide-PEG2000, or Chol-PEG2000.

In one embodiment, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O—(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In one embodiment, the polymer conjugated lipid is present in a concentration ranging from 1.0 to 2.5 molar percent. In one embodiment, the polymer conjugated lipid is present in a concentration of about 1.7 molar percent. In one embodiment, the polymer conjugated lipid is present in a concentration of about 1.5 molar percent.

In one embodiment, the molar ratio of cationic lipid to the polymer conjugated lipid ranges from about 35:1 to about 25:1. In one embodiment, the molar ratio of cationic lipid to polymer conjugated lipid ranges from about 100:1 to about 20:1.

In one embodiment, the molar ratio of cationic lipid to the polymer conjugated lipid ranges from about 35:1 to about 25:1. In one embodiment, the molar ratio of cationic lipid to polymer conjugated lipid ranges from about 100:1 to about 20:1.

In one embodiment, the pegylated lipid has the following Formula:

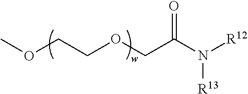

or a pharmaceutically acceptable salt, tautomer or stereoisomer thereof, wherein:
R¹² and R³ are each independently a straight or branched, saturated or unsaturated alkyl chain containing from 10 to 30 carbon atoms, wherein the alkyl chain is optionally interrupted by one or more ester bonds; and
w has a mean value ranging from 30 to 60.

In one embodiment, R¹² and R¹³ are each independently straight, saturated alkyl chains containing from 12 to 16 carbon atoms. In other embodiments, the average w ranges from 42 to 55, for example, the average w is 42, 43, 44, 45, 46, 47, 48, 49, 50, 51, 52, 53, 54 or 55. In some specific embodiments, the average w is about 49.

In one embodiment, the pegylated lipid has the following Formula:

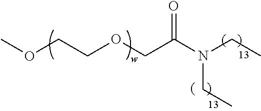

wherein the average w is about 49.

### 5.3.5 Structural Lipids

In some embodiments, the lipid component of a sphingomyelin-containing composition, nanoparticle composition, or nanoparticles provided herein can include one or more structural lipids. Without being bound by the theory, it is contemplated that structural lipids can stabilize the amphiphilic structure of a nanoparticle, such as but not limited to the lipid bilayer structure of a nanoparticle. Exemplary structural lipids that can be used in connection with the present disclosure include but are not limited to cholesterol, fecosterol, sitosterol, ergosterol, campesterol, stigmasterol, brassicasterol, tomatidine, tomatine, ursolic acid, alpha-tocopherol, and mixtures thereof. In certain embodiments, the structural lipid is cholesterol. In some embodiments, the structural lipid includes cholesterol and a corticosteroid (such as prednisolone, dexamethasone, prednisone, and hydrocortisone), or a combination thereof.

In one embodiment, the lipid nanoparticles provided herein comprise a steroid or steroid analogue. In one embodiment, the steroid or steroid analogue is cholesterol. In one embodiment, the steroid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent.

In one embodiment, the molar ratio of cationic lipid to the steroid ranges from 1.0:0.9 to 1.0:1.2, or from 1.0:1.0 to 1.0:1.2. In one embodiment, the molar ratio of cationic lipid to cholesterol ranges from about 5:1 to 1:1. In one embodiment, the steroid is present in a concentration ranging from 32 to 40 mol percent of the steroid.

In one embodiment, the molar ratio of cationic lipid to the steroid ranges from 1.0:0.9 to 1.0:1.2, or from 1.0:1.0 to 1.0:1.2. In one embodiment, the molar ratio of cationic lipid to cholesterol ranges from about 5:1 to 1:1. In one embodiment, the steroid is present in a concentration ranging from 32 to 40 mol percent of the steroid.

### 5.3.6 Phospholipids

In some embodiments, the lipid component of a sphingomyelin-containing composition, nanoparticle composition, or nanoparticles provided herein can include one or more phospholipids, such as one or more (poly)unsaturated lipids. Without being bound by the theory, it is contemplated that phospholipids may assemble into one or more lipid bilayers structures. Exemplary phospholipids that can form part of the present nanoparticle composition include but are not limited to 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In certain embodiments, a nanoparticle composition includes DSPC. In certain embodiments, a nanoparticle composition includes DOPE. In some embodiments, a nanoparticle composition includes both DSPC and DOPE.

Additional exemplary neutral lipids include, for example, dipalmitoylphosphatidylglycerol (DPPG), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC). In one embodiment, the neutral lipid is selected from DSPC, DPPC, DMPC, DOPC, POPC, DOPE and SM.

In one embodiment, the neutral lipid is phosphatidylcholine (PC), phosphatidylethanolamine (PE) phosphatidylserine (PS), phosphatidic acid (PA), or phosphatidylglycerol (PG).

Additionally phospholipids that can form part of the present nanoparticle composition also include those described in WO2017/112865, the entire content of which is hereby incorporated by reference in its entirety.

### 5.3.7 Therapeutic Payload

According to the present disclosure, a sphingomyelin-containing composition, nanoparticle composition, or nanoparticles provided herein can further comprise one or more therapeutic and/or prophylactic agents. These therapeutic and/or prophylactic agents are sometimes referred to as a “therapeutic payload” or “payload” in the present disclosure. In some embodiments, the therapeutic payload can be administered in vivo or in vitro using the nanoparticles as a delivery vehicle.

In some embodiments, the nanoparticle composition comprises, as the therapeutic payload, a small molecule compound (e.g., a small molecule drug) such as antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cytosine arabinoside, anthracyclines, alkylating agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetalol), antihypertensive agents (e.g., clonidine and hydralazine), anti-depressants (e.g., imipramine, amitriptyline, and doxepin), anti-convulsants (e.g., phenytoin), antihistamines (e.g., diphenhydramine, chlorpheniramine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some embodiments, the therapeutic payload comprises a cytotoxin, a radioactive ion, a chemotherapeutic, a vaccine, a compound that elicits an immune response, and/or another therapeutic and/or prophylactic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol, rachelmycin (CC-1065), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium.

In other embodiments, the therapeutic payload of the present nanoparticle composition can include, but is not limited to, therapeutic and/or prophylactic agents such as antimetabolites (e.g., methotrexate, 6-mercaptopurine, 6-thioguanine, cytarabine, 5-fluorouracil, dacarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the nanoparticle composition comprises, as the therapeutic payload, a biological molecule such as peptides and polypeptides. The biological molecules forming part of the present nanoparticle composition can be either of a natural source or synthetic. For example, in some embodiments, the therapeutic payload of the present nanoparticle composition can include, but is not limited to gentamycin, amikacin, insulin, erythropoietin (EPO), granulocyte-colony stimulating factor (G-CSF), granulocyte-macrophage colony stimulating factor (GM-CSF), Factor VIR, luteinizing hormone-releasing hormone (LHRH) analogs, interferons, heparin, Hepatitis B surface antigen, typhoid vaccine, cholera vaccine, and peptides and polypeptides.

### 5.3.7.1 Nucleic Acids

In some embodiments, the present nanoparticle composition comprises one or more nucleic acid molecules (e.g., DNA or RNA molecules) as the therapeutic payload. Exemplary forms of nucleic acid molecules that can be included in the present nanoparticle composition as therapeutic payload include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, the therapeutic payload comprises an RNA. RNA molecules that can be included in the present nanoparticle composition as the therapeutic payload include, but are not limited to, shortmers, agomirs, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and other forms of RNA molecules known in the art. In particular embodiments, the RNA is an mRNA.

In other embodiments, the nanoparticle composition comprises a siRNA molecule as the therapeutic payload. Particularly, in some embodiments, the siRNA molecule is capable of selectively interfering with and downregulate the expression of a gene of interest. For example, in some embodiments, the siRNA payload selectively silence a gene associated with a particular disease, disorder, or condition upon administration to a subject in need thereof of a nanoparticle composition including the siRNA. In some embodiments, the siRNA molecule comprises a sequence that is complementary to an mRNA sequence encoding a protein product of interest. In some embodiments, the siRNA molecule is an immunomodulatory siRNA.

In some embodiments, the nanoparticle composition comprises a shRNA molecule or a vector encoding the shRNA molecule as the therapeutic payload. Particularly, in some embodiments, the therapeutic payload, upon administering to a target cell, produces shRNA inside the target cell. Constructs and mechanisms relating to shRNA are well known in the relevant arts.

In some embodiments, the nanoparticle composition comprises an mRNA molecule as the therapeutic payload. Particularly, in some embodiments, the mRNA molecule encodes a polypeptide of interest, including any naturally or non-naturally occurring or otherwise modified polypeptide. A polypeptide encoded by an mRNA may be of any size and may have any secondary structure or activity. In some embodiments, the polypeptide encoded by an mRNA payload can have a therapeutic effect when expressed in a cell.

In some embodiment, a nucleic acid molecule of the present disclosure comprises an mRNA molecule. In specific embodiments, the nucleic acid molecule comprises at least one coding region encoding a peptide or polypeptide of interest (e.g., an open reading frame (ORF)). In some embodiments, the nucleic acid molecule further comprises at least one untranslated region (UTR). In particular embodiments, the untranslated region (UTR) is located upstream (to the 5′-end) of the coding region, and is referred to herein as the 5′-UTR. In particular embodiments, the untranslated region (UTR) is located downstream (to the 3′-end) of the coding region, and is referred to herein as the 3′-UTR. In particular embodiments, the nucleic acid molecule comprises both a 5′-UTR and a 3′-UTR. In some embodiments, the 5′-UTR comprises a 5′-Cap structure. In some embodiments, the nucleic acid molecule comprises a Kozak sequence (e.g., in the 5′-UTR). In some embodiments, the nucleic acid molecule comprises a poly-A region (e.g., in the 3′-UTR). In some embodiments, the nucleic acid molecule comprises a polyadenylation signal (e.g., in the 3′-UTR). In some embodiments, the nucleic acid molecule comprises stabilizing region (e.g., in the 3′-UTR). In some embodiments, the nucleic acid molecule comprises a secondary structure. In some embodiments, the secondary structure is a stem-loop. In some embodiments, the nucleic acid molecule comprises a stem-loop sequence (e.g., in the 5′-UTR and/or the 3′-UTR). In some embodiments, the nucleic acid molecule comprises one or more intronic regions capable of being excised during splicing. In a specific embodiment, the nucleic acid molecule comprises one or more region selected from a 5′-UTR, and a coding region. In a specific embodiment, the nucleic acid molecule comprises one or more region selected from a coding region and a 3′-UTR. In a specific embodiment, the nucleic acid molecule comprises one or more region selected from a 5′-UTR, a coding region, and a 3′-UTR.

### Coding Region

In some embodiments, the nucleic acid molecule of the present disclosure comprises at least one coding region. In some embodiments, the coding region is an open reading frame (ORF) that encodes for a single peptide or protein. In some embodiments, the coding region comprises at least two ORFs, each encoding a peptide or protein. In those embodiments where the coding region comprises more than one ORFs, the encoded peptides and/or proteins can be the same as or different from each other. In some embodiments, the multiple ORFs in a coding region are separated by non-coding sequences. In specific embodiments, a non-coding sequence separating two ORFs comprises an internal ribosome entry sites (IRES).

Without being bound by the theory, it is contemplated that an internal ribosome entry sites (IRES) can act as the sole ribosome binding site, or serve as one of multiple ribosome binding sites of an mRNA. An mRNA molecule containing more than one functional ribosome binding site can encode several peptides or polypeptides that are translated independently by the ribosomes (e.g., multicistronic mRNA). Accordingly, in some embodiments, the nucleic acid molecule of the present disclosure (e.g., mRNA) comprises one or more internal ribosome entry sites (IRES). Examples of IRES sequences that can be used in connection with the present disclosure include, without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In various embodiments, the nucleic acid molecule of the present disclose encodes for at least 1, 2, 3, 4, 5, 6, 7, 8, 9, 10 or more than 10 peptides or proteins. Peptides and proteins encoded by a nucleic acid molecule can be the same or different. In some embodiments, the nucleic acid molecule of the present disclosure encodes a dipeptide (e.g., camosine and anserine). In some embodiments, the nucleic acid molecule encodes a tripeptide. In some embodiments, the nucleic acid molecule encodes a tetrapeptide. In some embodiments, the nucleic acid molecule encodes a pentapeptide. In some embodiments, the nucleic acid molecule encodes a hexapeptide. In some embodiments, the nucleic acid molecule encodes a heptapeptide. In some embodiments, the nucleic acid molecule encodes an octapeptide. In some embodiments, the nucleic acid molecule encodes a nonapeptide. In some embodiments, the nucleic acid molecule encodes a decapeptide. In some embodiments, the nucleic acid molecule encodes a peptide or polypeptide that has at least about 15 amino acids. In some embodiments, the nucleic acid molecule encodes a peptide or polypeptide that has at least about 50 amino acids. In some embodiments, the nucleic acid molecule encodes a peptide or polypeptide that has at least about 100 amino acids. In some embodiments, the nucleic acid molecule encodes a peptide or polypeptide that has at least about 150 amino acids. In some embodiments, the nucleic acid molecule encodes a peptide or polypeptide that has at least about 300 amino acids. In some embodiments, the nucleic acid molecule encodes a peptide or polypeptide that has at least about 500 amino acids. In some embodiments, the nucleic acid molecule encodes a peptide or polypeptide that has at least about 1000 amino acids.

In some embodiments, the nucleic acid molecule of the present disclosure is at least about 30 nucleotides (nt) in length. In some embodiments, the nucleic acid molecule is at least about 35 nt in length. In some embodiments, the nucleic acid molecule is at least about 40 nt in length. In some embodiments, the nucleic acid molecule is at least about 45 nt in length. In some embodiments the nucleic acid molecule is at least about 50 nt in length. In some embodiments, the nucleic acid molecule is at least about 55 nt in length. In some embodiments, the nucleic acid molecule is at least about 60 nt in length. In some embodiments, the nucleic acid molecule is at least about 65 nt in length. In some embodiments, the nucleic acid molecule is at least about 70 nt in length. In some embodiments, the nucleic acid molecule is at least about 75 nt in length. In some embodiments, the nucleic acid molecule is at least about 80 nt in length. In some embodiments the nucleic acid molecule is at least about 85 nt in length. In some embodiments, the nucleic acid molecule is at least about 90 nt in length. In some embodiments, the nucleic acid molecule is at least about 95 nt in length. In some embodiments, the nucleic acid molecule is at least about 100 nt in length. In some embodiments, the nucleic acid molecule is at least about 120 nt in length. In some embodiments, the nucleic acid molecule is at least about 140 nt in length. In some embodiments, the nucleic acid molecule is at least about 160 nt in length. In some embodiments, the nucleic acid molecule is at least about 180 nt in length. In some embodiments, the nucleic acid molecule is at least about 200 nt in length. In some embodiments, the nucleic acid molecule is at least about 250 nt in length. In some embodiments, the nucleic acid molecule is at least about 300 nt in length. In some embodiments, the nucleic acid molecule is at least about 400 nt in length. In some embodiments, the nucleic acid molecule is at least about 500 nt in length. In some embodiments, the nucleic acid molecule is at least about 600 nt in length. In some embodiments, the nucleic acid molecule is at least about 700 nt in length. In some embodiments, the nucleic acid molecule is at least about 800 nt in length. In some embodiments, the nucleic acid molecule is at least about 900 nt in length. In some embodiments, the nucleic acid molecule is at least about 1000 nt in length. In some embodiments, the nucleic acid molecule is at least about 1100 nt in length. In some embodiments, the nucleic acid molecule is at least about 1200 nt in length. In some embodiments, the nucleic acid molecule is at least about 1300 nt in length. In some embodiments, the nucleic acid molecule is at least about 1400 nt in length. In some embodiments, the nucleic acid molecule is at least about 1500 nt in length. In some embodiments, the nucleic acid molecule is at least about 1600 nt in length. In some embodiments, the nucleic acid molecule is at least about 1700 nt in length. In some embodiments, the nucleic acid molecule is at least about 1800 nt in length. In some embodiments, the nucleic acid molecule is at least about 1900 nt in length. In some embodiments, the nucleic acid molecule is at least about 2000 nt in length. In some embodiments, the nucleic acid molecule is at least about 2500 nt in length. In some embodiments, the nucleic acid molecule is at least about 3000 nt in length. In some embodiments, the nucleic acid molecule is at least about 3500 nt in length. In some embodiments, the nucleic acid molecule is at least about 4000 nt in length. In some embodiments, the nucleic acid molecule is at least about 4500 nt in length. In some embodiments, the nucleic acid molecule is at least about 5000 nt in length.

In specific embodiments, the therapeutic payload comprises a vaccine composition (e.g., a genetic vaccine) as described herein. In some embodiments, the therapeutic payload comprises a compound capable of eliciting immunity against one or more target conditions or disease. In some embodiments, the target condition is related to or caused by infection by a pathogen, such as a coronavirus (e.g. 2019-nCoV), influenza, measles, human papillomavirus (HPV), rabies, meningitis, whooping cough, tetanus, plague, hepatitis, and tuberculosis. In some embodiments, the therapeutic payload comprises a nucleic acid sequence (e.g., mRNA) encoding a pathogenic protein characteristic for the pathogen, or an antigenic fragment or epitope thereof. The vaccine, upon administration to a vaccinated subject, allows for expression of the encoded pathogenic protein (or the antigenic fragment or epitope thereof), thereby eliciting immunity in the subject against the pathogen.

In some embodiments, the target condition is related to or caused by neoplastic growth of cells, such as a cancer. In some embodiments, the therapeutic payload comprises a nucleic acid sequence (e.g., mRNA) encoding a tumor associated antigen (TAA) characteristic for the cancer, or an antigenic fragment or epitope thereof. The vaccine, upon administration to a vaccinated subject, allows for expression of the encoded TAA (or the antigenic fragment or epitope thereof), thereby eliciting immunity in the subject against the neoplastic cells expressing the TAA.

### 5′-Cap Structure

Without being bound by the theory, it is contemplated that, a 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The 5′-cap structure further assists the removal of 5′-proximal introns removal during mRNA splicing. Accordingly, in some embodiments, the nucleic acid molecules of the present disclosure comprise a 5′-cap structure.

Nucleic acid molecules may be 5′-end capped by the endogenous transcription machinery of a cell to generate a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a nucleic acid molecule, such as an mRNA molecule, for degradation.

In some embodiments, the nucleic acid molecules of the present disclosure comprise one or more alterations to the natural 5′-cap structure generated by the endogenous process. Without being bound by the theory, a modification on the 5′-cap may increase the stability of polynucleotide, increase the half-life of the polynucleotide, and could increase the polynucleotide translational efficiency.

Exemplary alterations to the natural 5′-Cap structure include generation of a non-hydrolyzable cap structure preventing decapping and thus increasing polynucleotide half-life. In some embodiments, because cap structure hydrolysis requires cleavage of 5′-ppp-5′ phosphorodiester linkages, in some embodiments, modified nucleotides may be used during the capping reaction. For example, in some embodiments, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used, such as α-methyl-phosphonate and seleno-phosphate nucleotides.

Additional exemplary alterations to the natural 5′-Cap structure also include modification at the 2′- and/or 3′-position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

Additional exemplary alterations to the natural 5′-cap structure include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule. Additional exemplary 5′-Cap structures that can be used in connection with the present disclosure further include those described in International Patent Publication Nos. WO2008127688, WO 2008016473, and WO 2011015347, the entire contents of each of which are incorporated herein by reference.

In various embodiments, 5′-terminal caps can include cap analogs. Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,3′-O-dimethyl-guanosine-5′-triphosphate-5′-guanosine, m⁷G-3′mppp-G, which may equivalently be designated 3′ 0-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methlyated guanosine provides the terminal moiety of the capped polynucleotide (e.g., mRNA). Another exemplary cap structure is mCAP, which is similar to ARCA but has a 2′-O-methyl group on guanosine (i.e., N7,2′-O-dimethyl-guanosine-5′-triphosphate-5′-guanosine, m₇Gm-ppp-G).

In some embodiments, a cap analog can be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the entire content of which is herein incorporated by reference in its entirety.

In some embodiments, a cap analog can be a N7-(4-chlorophenoxyethyl) substituted dinucleotide cap analog known in the art and/or described herein. Non-limiting examples of N7-(4-chlorophenoxyethyl) substituted dinucleotide cap analogs include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574: the entire content of which is herein incorporated by reference). In other embodiments, a cap analog useful in connection with the nucleic acid molecules of the present disclosure is a 4-chloro/bromophenoxyethyl analog.

In various embodiments, a cap analog can include a guanosine analog. Useful guanosine analogs include but are not limited to inosine, N1-methyl-guanosine, 2′-fluoro-guanosine, 7-deaza-guanosine, 8-oxo-guanosine, 2-amino-guanosine, LNA-guanosine, and 2-azido-guanosine.

Without being bound by the theory, it is contemplated that while cap analogs allow for the concomitant capping of a polynucleotide in an in vitro transcription reaction, up to 20% of transcripts remain uncapped. This, as well as the structural differences of a cap analog from the natural 5′-cap structures of polynucleotides produced by the endogenous transcription machinery of a cell, may lead to reduced translational competency and reduced cellular stability.

Accordingly, in some embodiments, a nucleic acid molecule of the present disclosure can also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in connection with the nucleic acid molecules of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, in some embodiments, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap1 structure. This cap results in a higher translational-competency, cellular stability, and a reduced activation of cellular pro-inflammatory cytokines, as compared, e.g., to other 5′ cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NmpN2mp (Cap 2), and m(7)Gpppm(3)(6,6,2′)Apm(2′)Apm(2′)Cpm(2)(3,2′)Up (Cap 4).

Without being bound by the theory, it is contemplated that the nucleic acid molecules of the present disclosure can be capped post-transcriptionally, and because this process is more efficient, nearly 100% of the nucleic acid molecules may be capped.

### Untranslated Regions (UTRs)

In some embodiments, the nucleic acid molecules of the present disclosure comprise one or more untranslated regions (UTRs). In some embodiments, an UTR is positioned upstream to a coding region in the nucleic acid molecule, and is termed 5′-UTR. In some embodiments, an UTR is positioned downstream to a coding region in the nucleic acid molecule, and is termed 3′-UTR. The sequence of an UTR can be homologous or heterologous to the sequence of the coding region found in a nucleic acid molecule. Multiple UTRs can be included in a nucleic acid molecule and can be of the same or different sequences, and/or genetic origin. According to the present disclosure, any portion of UTRs in a nucleic acid molecule (including none) can be codon optimized and any may independently contain one or more different structural or chemical modification, before and/or after codon optimization.

In some embodiments, a nucleic acid molecule of the present disclosure (e.g., mRNA) comprises UTRs and coding regions that are homologous with respect to each other. In other embodiments, a nucleic acid molecule of the present disclosure (e.g., mRNA) comprises UTRs and coding regions that are heterogeneous with respect to each other. In some embodiments, to monitor the activity of a UTR sequence, a nucleic acid molecule comprising the UTR and a coding sequence of a detectable probe can be administered in vitro (e.g., cell or tissue culture) or in vivo (e.g., to a subject), and an effect of the UTR sequence (e.g., modulation on the expression level, cellular localization of the encoded product, or half-life of the encoded product) can be measured using methods known in the art.

In some embodiments, the UTR of a nucleic acid molecule of the present disclosure (e.g., mRNA) comprises at least one translation enhancer element (TEE) that functions to increase the amount of polypeptide or protein produced from the nucleic acid molecule. In some embodiments, the TEE is located in the 5′-UTR of the nucleic acid molecule. In other embodiments, the TEE is located at the 3′-UTR of the nucleic acid molecule. In yet other embodiments, at least two TEE are located at the 5′-UTR and 3′-UTR of the nucleic acid molecule respectively. In some embodiments, a nucleic acid molecule of the present disclosure (e.g., mRNA) can comprise one or more copies of a TEE sequence or comprise more than one different TEE sequences. In some embodiments, different TEE sequences that are present in a nucleic acid molecule of the present disclosure can be homologues or heterologous with respect to one another.

Various TEE sequences that are known in the art and can be used in connection with the present disclosure. For example, in some embodiments, the TEE can be an internal ribosome entry site (IRES), HCV-IRES or an IRES element. Chappell et al. Proc. Natl. Acad. Sci. USA 101:9590-9594, 2004; Zhou et al. Proc. Natl. Acad. Sci. 102:6273-6278, 2005. Additional internal ribosome entry site (IRES) that can be used in connection with the present disclosure include but are not limited to those described in U.S. Pat. No. 7,468,275, U.S. Patent Publication No. 2007/0048776 and U.S. Patent Publication No. 2011/0124100 and International Patent Publication No. WO2007/025008 and International Patent Publication No. WO2001/055369, the content of each of which is enclosed herein by reference in its entirety. In some embodiments, the TEE can be those described in Supplemental Table 1 and in Supplemental Table 2 of Wellensiek et al Genome-wide profiling of human cap-independent translation-enhancing elements, Nature Methods, 2013 August; 10(8): 747-750: the content of which is incorporated by reference in its entirety.

Additional exemplary TEEs that can be used in connection with the present disclosure include but are not limited to the TEE sequences disclosed in U.S. Pat. Nos. 6,310,197, 6,849,405, 7,456,273, 7,183,395, U.S. Patent Publication No. 2009/0226470, U.S. Patent Publication No. 2013/0177581, U.S. Patent Publication No. 2007/0048776, U.S. Patent Publication No. 2011/0124100, U.S. Patent Publication No. 2009/0093049, International Patent Publication No. WO2009/075886, International Patent Publication No. WO2012/009644, and International Patent Publication No. WO1999/024595, International Patent Publication No. WO2007/025008, International Patent Publication No. WO2001/055371, European Patent No. 2610341, European Patent No. 2610340, the content of each of which is enclosed herein by reference in its entirety.

In various embodiments, a nucleic acid molecule of the present disclosure (e.g., mRNA) comprises at least one UTR that comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some embodiments, the TEE sequences in the UTR of a nucleic acid molecule are copies of the same TEE sequence. In other embodiments, at least two TEE sequences in the UTR of a nucleic acid molecule are of different TEE sequences. In some embodiments, multiple different TEE sequences are arranged in one or more repeating patterns in the UTR region of a nucleic acid molecule. For illustrating purpose only, a repeating pattern can be, for example, ABABAB, AABBAABBAABB, ABCABCABC, or the like, where in these exemplary patterns, each capitalized letter (A, B, or C) represents a different TEE sequence. In some embodiments, at least two TEE sequences are consecutive with one another (i.e., no spacer sequence in between) in a UTR of a nucleic acid molecule. In other embodiments, at least two TEE sequences are separated by a spacer sequence. In some embodiments, a UTR can comprise a TEE sequence-spacer sequence module that is repeated at least once, at least twice, at least 3 times, at least 4 times, at least 5 times, at least 6 times, at least 7 times, at least 8 times, at least 9 times, or more than 9 times in the UTR. In any of the embodiments described in this paragraph, the UTR can be a 5′-UTR, a 3′-UTR or both 5′-UTR and 3′-UTR of a nucleic acid molecule.

In some embodiments, the UTR of a nucleic acid molecule of the present disclosure (e.g., mRNA) comprises at least one translation suppressing element that functions to decrease the amount of polypeptide or protein produced from the nucleic acid molecule. In some embodiments, the UTR of the nucleic acid molecule comprises one or more miR sequences or fragment thereof (e.g., miR seed sequences) that are recognized by one or more microRNA. In some embodiments, the UTR of the nucleic acid molecule comprises one or more stem-loop structure that downregulates translational activity of the nucleic acid molecule. Other mechanisms for suppressing translational activities associated with a nucleic acid molecules are known in the art. In any of the embodiments described in this paragraph, the UTR can be a 5′-UTR, a 3′-UTR or both 5′-UTR and 3′-UTR of a nucleic acid molecule.

### The Polyadenylation (Poly-A) Regions

During natural RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Without being bound by the theory, it is contemplated that a poly-A region can confer various advantages to the nucleic acid molecule of the present disclosure.

Accordingly, in some embodiments, a nucleic acid molecule of the present disclosure (e.g., an mRNA) comprises a polyadenylation signal. In some embodiments, a nucleic acid molecule of the present disclosure (e.g., an mRNA) comprises one or more polyadenylation (poly-A) regions. In some embodiments, a poly-A region is composed entirely of adenine nucleotides or functional analogs thereof. In some embodiments, the nucleic acid molecule comprises at least one poly-A region at its 3′-end. In some embodiments, the nucleic acid molecule comprises at least one poly-A region at its 5′-end. In some embodiments, the nucleic acid molecule comprises at least one poly-A region at its 5′-end and at least one poly-A region at its 3′-end.

According to the present disclosure, the poly-A region can have varied lengths in different embodiments. Particularly, in some embodiments, the poly-A region of a nucleic acid molecule of the present disclosure is at least 30 nucleotides in length. In some embodiments, the poly-A region of a nucleic acid molecule of the present disclosure is at least 35 nucleotides in length. In some embodiments, the poly-A region of a nucleic acid molecule of the present disclosure is at least 40 nucleotides in length. In some embodiments, the poly-A region of a nucleic acid molecule of the present disclosure is at least 45 nucleotides in length. In some embodiments, the poly-A region of a nucleic acid molecule of the present disclosure is at least 50 nucleotides in length. In some embodiments, the poly-A region of a nucleic acid molecule of the present disclosure is at least 55 nucleotides in length. In some embodiments, the poly-A region of a nucleic acid molecule of the present disclosure is at least 60 nucleotides in length. In some embodiments, the poly-A region of a nucleic acid molecule of the present disclosure is at least 65 nucleotides in length. In some embodiments, the poly-A region of a nucleic acid molecule of the present disclosure is at least 70 nucleotides in length. In some embodiments, the poly-A region of a nucleic acid molecule of the present disclosure is at least 75 nucleotides in length. In some embodiments, the poly-A region of a nucleic acid molecule of the present disclosure is at least 80 nucleotides in length. In some embodiments, the poly-A region of a nucleic acid molecule of the present disclosure is at least 85 nucleotides in length. In some embodiments, the poly-A region of a nucleic acid molecule of the present disclosure is at least 90 nucleotides in length. In some embodiments, the poly-A region of a nucleic acid molecule of the present disclosure is at least 95 nucleotides in length. In some embodiments, the poly-A region of a nucleic acid molecule of the present disclosure is at least 100 nucleotides in length. In some embodiments, the poly-A region of a nucleic acid molecule of the present disclosure is at least 110 nucleotides in length. In some embodiments, the poly-A region of a nucleic acid molecule of the present disclosure is at least 120 nucleotides in length. In some embodiments, the poly-A region of a nucleic acid molecule of the present disclosure is at least 130 nucleotides in length. In some embodiments, the poly-A region of a nucleic acid molecule of the present disclosure is at least 140 nucleotides in length. In some embodiments, the poly-A region of a nucleic acid molecule of the present disclosure is at least 150 nucleotides in length. In some embodiments, the poly-A region of a nucleic acid molecule of the present disclosure is at least 160 nucleotides in length. In some embodiments, the poly-A region of a nucleic acid molecule of the present disclosure is at least 170 nucleotides in length. In some embodiments, the poly-A region of a nucleic acid molecule of the present disclosure is at least 180 nucleotides in length. In some embodiments, the poly-A region of a nucleic acid molecule of the present disclosure is at least 190 nucleotides in length. In some embodiments, the poly-A region of a nucleic acid molecule of the present disclosure is at least 200 nucleotides in length. In some embodiments, the poly-A region of a nucleic acid molecule of the present disclosure is at least 225 nucleotides in length. In some embodiments, the poly-A region of a nucleic acid molecule of the present disclosure is at least 250 nucleotides in length. In some embodiments, the poly-A region of a nucleic acid molecule of the present disclosure is at least 275 nucleotides in length. In some embodiments, the poly-A region of a nucleic acid molecule of the present disclosure is at least 300 nucleotides in length. In some embodiments, the poly-A region of a nucleic acid molecule of the present disclosure is at least 350 nucleotides in length. In some embodiments, the poly-A region of a nucleic acid molecule of the present disclosure is at least 400 nucleotides in length. In some embodiments, the poly-A region of a nucleic acid molecule of the present disclosure is at least 450 nucleotides in length. In some embodiments, the poly-A region of a nucleic acid molecule of the present disclosure is at least 500 nucleotides in length. In some embodiments, the poly-A region of a nucleic acid molecule of the present disclosure is at least 600 nucleotides in length. In some embodiments, the poly-A region of a nucleic acid molecule of the present disclosure is at least 700 nucleotides in length. In some embodiments, the poly-A region of a nucleic acid molecule of the present disclosure is at least 800 nucleotides in length. In some embodiments, the poly-A region of a nucleic acid molecule of the present disclosure is at least 900 nucleotides in length. In some embodiments, the poly-A region of a nucleic acid molecule of the present disclosure is at least 1000 nucleotides in length. In some embodiments, the poly-A region of a nucleic acid molecule of the present disclosure is at least 1100 nucleotides in length. In some embodiments, the poly-A region of a nucleic acid molecule of the present disclosure is at least 1200 nucleotides in length. In some embodiments, the poly-A region of a nucleic acid molecule of the present disclosure is at least 1300 nucleotides in length. In some embodiments, the poly-A region of a nucleic acid molecule of the present disclosure is at least 1400 nucleotides in length. In some embodiments, the poly-A region of a nucleic acid molecule of the present disclosure is at least 1500 nucleotides in length. In some embodiments, the poly-A region of a nucleic acid molecule of the present disclosure is at least 1600 nucleotides in length. In some embodiments, the poly-A region of a nucleic acid molecule of the present disclosure is at least 1700 nucleotides in length. In some embodiments, the poly-A region of a nucleic acid molecule of the present disclosure is at least 1800 nucleotides in length. In some embodiments, the poly-A region of a nucleic acid molecule of the present disclosure is at least 1900 nucleotides in length. In some embodiments, the poly-A region of a nucleic acid molecule of the present disclosure is at least 2000 nucleotides in length. In some embodiments, the poly-A region of a nucleic acid molecule of the present disclosure is at least 2250 nucleotides in length. In some embodiments, the poly-A region of a nucleic acid molecule of the present disclosure is at least 2500 nucleotides in length. In some embodiments, the poly-A region of a nucleic acid molecule of the present disclosure is at least 2750 nucleotides in length. In some embodiments, the poly-A region of a nucleic acid molecule of the present disclosure is at least 3000 nucleotides in length.

In some embodiments, length of a poly-A region in a nucleic acid molecule can be selected based on the overall length of the nucleic acid molecule, or a portion thereof (such as the length of the coding region or the length of an open reading frame of the nucleic acid molecule, etc.). For example, in some embodiments, the poly-A region accounts for about 5%, 10%, 15%, 20%, 25%, 30%, 35%, 40%, 45%, 50%, 55%, 60%, 65%, 70%, 75%, 80%, 85%, 90%, 95% or more of the total length of nucleic acid molecule containing the poly-A region.

Without being bound by the theory, it is contemplated that certain RNA-binding proteins can bind to the poly-A region located at the 3′-end of an mRNA molecule. These poly-A binding proteins (PABP) can modulate mRNA expression, such as interacting with translation initiation machinery in a cell and/or protecting the 3′-poly-A tails from degradation. Accordingly, in some embodiments, in some embodiments, the nucleic acid molecule of the present disclosure (e.g., mRNA) comprises at least one binding site for poly-A binding protein (PABP). In other embodiments, the nucleic acid molecule is conjugated or complex with a PABP before loaded into a delivery vehicle (e.g., lipid nanoparticles).

In some embodiments, the nucleic acid molecule of the present disclosure (e.g., mRNA) comprises a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet structure results in protein production equivalent to at least 75% of that seen using a poly-A region of 120 nucleotides alone.

In some embodiments, the nucleic acid molecule of the present disclosure (e.g., mRNA) may include a poly-A region and may be stabilized by the addition of a 3′-stabilizing region. In some embodiments, the 3′-stabilizing region which may be used to stabilize a nucleic acid molecule (e.g., mRNA) including the poly-A or poly-A-G Quartet structures as described in International Patent Publication No. WO2013/103659, the content of which is incorporated herein by reference in its entirety.

In other embodiments, the 3′-stabilizing region which may be used in connection with the nucleic acid molecules of the present disclosure include a chain termination nucleoside such as but is not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

### Secondary Structure

Without being bound by the theory, it is contemplated that a stem-loop structure can direct RNA folding, protect structural stability of a nucleic acid molecule (e.g., mRNA), provide recognition sites for RNA binding proteins, and serve as a substrate for enzymatic reactions. For example, the incorporation of a miR sequence and/or a TEE sequence changes the shape of the stem loop region which may increase and/or decrease translation (Kedde et al. A Pumilio-induced RNA structure switch in p27-3′UTR controls miR-221 and miR-222 accessibility. Nat Cell Biol., 2010 October; 12(10):1014-20, the content of which is herein incorporated by reference in its entirety).

Accordingly, in some embodiments, the nucleic acid molecules as described herein (e.g., mRNA) or a portion thereof may assume a stem-loop structure, such as but is not limited to a histone stem loop. In some embodiments, the stem-loop structure is formed from a stem-loop sequence that is about 25 or about 26 nucleotides in length such as, but not limited to, those as described in International Patent Publication No. WO2013/103659, the content of which is incorporated herein by reference in its entirety. Additional examples of stem-loop sequences include those described in International Patent Publication No. WO2012/019780 and International Patent Publication No. WO201502667, the contents of which are incorporated herein by reference. In some embodiments, the step-loop sequence comprises a TEE as described herein. In some embodiments, the step-loop sequence comprises a miR sequence as described herein. In specific embodiments, the stem loop sequence may include a miR-122 seed sequence. In specific embodiments, the nucleic acid molecule comprises the two stem-loop sequence described in International Patent Publication No. WO2021204175, the entirety of which is incorporated herein by reference.

In some embodiments, the nucleic acid molecule of the present disclosure (e.g., mRNA) comprises a stem-loop sequence located upstream (to the 5′-end) of the coding region in a nucleic acid molecule. In some embodiments, the stem-loop sequence is located within the 5′-UTR of the nucleic acid molecule. In some embodiments, the nucleic acid molecule of the present disclosure (e.g., mRNA) comprises a stem-loop sequence located downstream (to the 3′-end) of the coding region in a nucleic acid molecule. In some embodiments, the stem-loop sequence is located within the 3′-UTR of the nucleic acid molecule. In some cases, a nucleic acid molecule can contain more than one stem-loop sequences. In some embodiment, the nucleic acid molecule comprises at least one stem-loop sequence in the 5′-UTR, and at least one stem-loop sequence in the 3′-UTR.

In some embodiments, a nucleic acid molecule comprising a stem-loop structure further comprises a stabilization region. In some embodiment, the stabilization region comprises at least one chain terminating nucleoside that functions to slow down degradation and thus increases the half-life of the nucleic acid molecule. Exemplary chain terminating nucleoside that can be used in connection with the present disclosure include but are not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein. In other embodiments, a stem-loop structure may be stabilized by an alteration to the 3′-region of the polynucleotide that can prevent and/or inhibit the addition of oligio(U) (International Patent Publication No. WO2013/103659, incorporated herein by reference in its entirety).

In some embodiments, a nucleic acid molecule of the present disclosure comprises at least one stem-loop sequence and a poly-A region or polyadenylation signal. Non-limiting examples of polynucleotide sequences comprising at least one stem-loop sequence and a poly-A region or a polyadenylation signal include those described in International Patent Publication No. WO2013/120497, International Patent Publication No. WO2013/120629, International Patent Publication No. WO2013/120500, International Patent Publication No. WO2013/120627, International Patent Publication No. WO2013/120498, International Patent Publication No. WO2013/120626, International Patent Publication No. WO2013/120499 and International Patent Publication No. WO2013/120628, the content of each of which is incorporated herein by reference in its entirety.

In some embodiments, the nucleic acid molecule comprising a stem-loop sequence and a poly-A region or a polyadenylation signal can encode for a pathogen antigen or fragment thereof such as the polynucleotide sequences described in International Patent Publication No. WO2013/120499 and International Patent Publication No. WO2013/120628, the content of each of which is incorporated herein by reference in its entirety.

In some embodiments, the nucleic acid molecule comprising a stem-loop sequence and a poly-A region or a polyadenylation signal can encode for a therapeutic protein such as the polynucleotide sequences described in International Patent Publication No. WO2013/120497 and International Patent Publication No. WO2013/120629, the content of each of which is incorporated herein by reference in its entirety.

In some embodiments, the nucleic acid molecule comprising a stem-loop sequence and a poly-A region or a polyadenylation signal can encode for a tumor antigen or fragment thereof such as the polynucleotide sequences described in International Patent Publication No. WO2013/120500 and International Patent Publication No. WO2013/120627, the content of each of which is incorporated herein by reference in its entirety.

In some embodiments, the nucleic acid molecule comprising a stem-loop sequence and a poly-A region or a polyadenylation signal can code for an allergenic antigen or an autoimmune self-antigen such as the polynucleotide sequences described in International Patent Publication No. WO2013/120498 and International Patent Publication No. WO2013/120626, the content of each of which is incorporated herein by reference in its entirety.

### Functional Nucleotide Analogs

In some embodiments, a payload nucleic acid molecule described herein contains only canonical nucleotides selected from A (adenosine), G (guanosine), C (cytosine), U (uridine), and T (thymidine). Without being bound by the theory, it is contemplated that certain functional nucleotide analogs can confer useful properties to a nucleic acid molecule. Examples of such as useful properties in the context of the present disclosure include but are not limited to increased stability of the nucleic acid molecule, reduced immunogenicity of the nucleic acid molecule in inducing innate immune responses, enhanced production of protein encoded by the nucleic acid molecule, increased intracellular delivery and/or retention of the nucleic acid molecule, and/or reduced cellular toxicity of the nucleic acid molecule, etc.

Accordingly, in some embodiments, a payload nucleic acid molecule comprises at least one functional nucleotide analog as described herein. In some embodiments, the functional nucleotide analog contains at least one chemical modification to the nucleobase, the sugar group and/or the phosphate group. Accordingly, a payload nucleic acid molecule comprising at least one functional nucleotide analog contains at least one chemical modification to the nucleobases, the sugar groups, and/or the internucleoside linkage. Exemplary chemical modifications to the nucleobases, sugar groups, or internucleoside linkages of a nucleic acid molecule are provided herein.

As described herein, ranging from 0% to 100% of all nucleotides in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of all nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

As described herein, ranging from 0% to 100% of all nucleotides of a kind (e.g., all purine-containing nucleotides as a kind, or all pyrimidine-containing nucleotides as a kind, or all A, G, C, T or U as a kind) in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of a kind of nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

### Modification to Nucleobases

In some embodiments, a functional nucleotide analog contains a non-canonical nucleobase. In some embodiments, canonical nucleobases (e.g., adenine, guanine, uracil, thymine, and cytosine) in a nucleotide can be modified or replaced to provide one or more functional analogs of the nucleotide. Exemplary modification to nucleobases include but are not limited to one or more substitutions or modifications including but not limited to alkyl, aryl, halo, oxo, hydroxyl, alkyloxy, and/or thio substitutions; one or more fused or open rings, oxidation, and/or reduction.

In some embodiments, the non-canonical nucleobase is a modified uracil. Exemplary nucleobases and nucleosides having an modified uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(τm⁵5s²U), 1-taurinomethyl-4-thio-pseudouridine, 5-methyl-uracil (m⁵U, i.e., having the nucleobase deoxythymine), 1-methyl-pseudouridine (m¹ψ), 1-ethyl-pseudouridine (Et¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (m⁵U), 5-(isopentenylaminomethyl)-2-thio-uracil (m⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the non-canonical nucleobase is a modified cytosine. Exemplary nucleobases and nucleosides having a modified cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (fMCm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the non-canonical nucleobase is a modified adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-azaadenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the non-canonical nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

In some embodiments, the non-canonical nucleobase of a functional nucleotide analog can be independently a purine, a pyrimidine, a purine or pyrimidine analog. For example, in some embodiments, the non-canonical nucleobase can be modified adenine, cytosine, guanine, uracil, or hypoxanthine. In other embodiments, the non-canonical nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

### Modification to the Sugar

In some embodiments, a functional nucleotide analog contains a non-canonical sugar group. In various embodiments, the non-canonical sugar group can be a 5-carbon or 6-carbon sugar (such as pentose, ribose, arabinose, xylose, glucose, galactose, or a deoxy derivative thereof) with one or more substitutions, such as a halo group, a hydroxy group, a thiol group, an alkyl group, an alkoxy group, an alkenyloxy group, an alkynyloxy group, an cycloalkyl group, an aminoalkoxy group, an alkoxyalkoxy group, an hydroxyalkoxy group, an amino group, an azido group, an aryl group, an aminoalkyl group, an aminoalkenyl group, an aminoalkynyl group, etc.

Generally, RNA molecules contains the ribose sugar group, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′+2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a nucleic acid molecule can include nucleotides containing, e.g., arabinose or L-ribose, as the sugar. In some embodiments, the nucleic acid molecule includes at least one nucleoside wherein the sugar is L-ribose, 2′-O-methyl-ribose, 2′-fluoro-ribose, arabinose, hexitol, an LNA, or a PNA.

### Modifications to the Internucleoside Linkage

In some embodiments, the payload nucleic acid molecule of the present disclosure can contain one or more modified internucleoside linkage (e.g., phosphate backbone). Backbone phosphate groups can be altered by replacing one or more of the oxygen atoms with a different substituent.

In some embodiments, the functional nucleotide analogs can include the replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy. The replacement of one or more of the oxygen atoms at the position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present disclosure, including internucleoside linkages which do not contain a phosphorous atom, are described herein.

Additional examples of nucleic acid molecules (e.g., mRNA), compositions, formulations and/or methods associated therewith that can be used in connection with the present disclosure further include those described in WO2002/098443, WO2003/051401, WO2008/052770, WO2009127230, WO2006122828, WO2008/083949, WO2010088927, WO2010/037539, WO2004/004743, WO2005/016376, WO2006/024518, WO2007/095976, WO2008/014979, WO2008/077592, WO2009/030481, WO2009/095226, WO2011069586, WO2011026641, WO2011/144358, WO2012019780, WO2012013326, WO2012089338, WO2012113513, WO2012116811, WO2012116810, WO2013113502, WO2013113501, WO2013113736, WO2013143698, WO2013143699, WO2013143700, WO2013/120626, WO2013120627, WO2013120628, WO2013120629, WO2013174409, WO2014127917, WO2015/024669, WO2015/024668, WO2015/024667, WO2015/024665, WO2015/024666, WO2015/024664, WO2015101415, WO2015101414, WO2015024667, WO2015062738, WO2015101416, the content of each of which is incorporated herein in its entirety.

### 5.3.8 Pharmaceutical Compositions

According to the present disclosure, nanoparticle compositions can be formulated in whole or in part as pharmaceutical compositions. Pharmaceutical compositions can include one or more nanoparticle compositions. For example, a pharmaceutical composition can include one or more nanoparticle compositions including one or more different therapeutic and/or prophylactic agents. Pharmaceutical compositions can further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients can be used in any pharmaceutical composition, except insofar as any conventional excipient or accessory ingredient can be incompatible with one or more components of a nanoparticle composition. An excipient or accessory ingredient can be incompatible with a component of a nanoparticle composition if its combination with the component can result in any undesirable biological effect or otherwise deleterious effect.

In some embodiments, one or more excipients or accessory ingredients can make up greater than 50% of the total mass or volume of a pharmaceutical composition including a nanoparticle composition. For example, the one or more excipients or accessory ingredients can make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100/o pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition can comprise between 0.1% and 100/6 (wt/wt) of one or more nanoparticle compositions.

In certain embodiments, the nanoparticle compositions and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising sphingomyelin and a compound of any of Formulae 01-I, 01-II, 02-I, 02-I, 03-I, 03-II-A, 03-II-B, 03-II-C, 03-II-D, 04-I, 04-III, 04-IV, 05-I and 06-I (and sub-formulas thereof) is a solution that is refrigerated for storage and/or shipment at, for example, about −20° C., −30° C., −40° C., −50° C., −60° C., −70° C., or −80° C. In certain embodiments, the disclosure also relates to a method of increasing stability of the nanoparticle compositions and/or pharmaceutical compositions comprising sphingomyelin and a compound of any of Formulae 01-I, 01-II, 02-I, 02-II, 03-I, 03-II-A, 03-II-B, 03-II-C, 03-II-D, 04-I, 04-II, 04-IV, 05-I, and 06-I (and sub-formulas thereof) by storing the nanoparticle compositions and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.). For example, the nanoparticle compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In one embodiment, the formulation is stabilized for at least 4 weeks at about 4° C. In certain embodiments, the pharmaceutical composition of the disclosure comprises a nanoparticle composition disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8, 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a nanoparticle composition disclosed herein, Tris, saline and sucrose, and has a pH of about 7.5-8, which is suitable for storage and/or shipment at, for example, about −20° C. For example, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

Nanoparticle compositions and/or pharmaceutical compositions including one or more nanoparticle compositions can be administered to any patient or subject, including those patients or subjects that can benefit from a therapeutic effect provided by the delivery of a therapeutic and/or prophylactic agent to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more nanoparticle compositions can be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions can be prepared in a variety of forms suitable for a variety of routes and methods of administration. For example, pharmaceutical compositions can be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactic agents, additional agents such as wetting agents, emulsifying and suspending agents, sweetening, flavoring, and/or perfuming agents. In certain embodiments for parenteral administration, compositions are mixed with solubilizing agents such as Cremophor™, alcohols, oils, modified oils, glycols, polysorbates, cyclodextrins, polymers, and/or combinations thereof.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

The disclosure features methods of delivering a therapeutic and/or prophylactic agent to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic agent.

### 6. EXAMPLES

The examples in this section (i.e., Section 6) are offered by way of illustration, and not by way of limitation.

### 6.1 Example 1: Preparation and Characterization of Lipid Nanoparticle Formulations

Briefly, the specified amount of the lipid components were solubilized in ethanol at the specified molar ratios (see Tables 6.2-1 to 6.2-3). The mRNA were diluted in 10 to 50 mM citrate buffer, pH=4. The LNPs were prepared at a total lipid to mRNA weight ratio of approximately 10:1 to 30:1 by mixing the ethanolic lipid solution with the aqueous mRNA solution at a volume ratio of 1:3 using a microfluidic apparatus, total flow rate ranging from 9-30 mL/min. Ethanol was thereby removed and replaced by DPBS using dialysis. Finally, the lipid nanoparticles were filtered through a 0.2 μm sterile filter.

Lipid nanoparticle size were determined by dynamic light scattering using a Malvern Zetasizer Nano ZS (Malvern UK) using a 1730 backscatter detection mode. The encapsulation efficiency of lipid nanoparticles was determined using a Quant-it Ribogreen RNA quantification assay kit (Thermo Fisher Scientific, UK) according to the manufacturer's instructions.

To measure the size and PDI of lipid nanoparticle, formulations were diluted 20-fold in PBS and transferred 1 mL in measurement cuvette. The LNP EE % was determined using a Quant-it RiboGreen RNA assay kit, LNP formulations were diluted to 0.4 μg/mL in Tris-EDTA and 0.1% Triton respectively. In order to determine free RNA and total RNA fluorescence intensity, ribogreen reagent were diluted 200-fold with Tris-EDTA buffer and mix at the same volume as diluted LNP formulation. Fluorescence intensity was measured at room temperature in a Molecular Devices Spectramax iD3 spectrometer using excitation and emission wavelengths of 488 nm and 525 nm. EE was calculated based on the ratio of encapsulated to total RNA fluorescence intensity.

### 6.2 Example 2: Physical Properties of Lipid Nanoparticle Formulations

Lipid nanoparticle formulations containing GFP gene and lipid compositions as listed in following Tables 6.2-1 to 6.2-3 were prepared, and physical characterization of the final LNP composition were performed according to the above methods.

TABLE 6.2-1
Physical characterization of LNP containing sphingomyelin.

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-1- | 45 (C1) | 0 | 10 | 43.5 | 1.5 | 71.4 | 0.059 | 90.5 |
| Control (lot 1) | | | | | | | | |
| Formulation-1- | 45 (C1) | 10 | 0 | 43.5 | 1.5 | 77.5 | 0.086 | 89.9 |
| SM (lot 2) | | | | | | | | |
| Formulation-1-BSM | 40 (C1) | 10 | 0 | 48.5 | 1.5 | 89.3 | 0.063 | 90.9 |
| Formulation-2-SM | 45 (C1) | 5 | 5 | 43.5 | 1.5 | 78.0 | 0.060 | 90.1 |
| Formulation-3-SM | 45 (C1) | 5 | 0 | 48.5 | 1.5 | 66.3 | 0.042 | 91.4 |

TABLE 6.2-2
Physical characterization of LNP containing
sphingomyelin at varied molar percentages.

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-1- | 45 (C1) | 0 | 10 | 43.5 | 1.5 | 70.2 | 0.038 | 93.1 |
| Control (lot 2) | | | | | | | | |
| Formulation-1- | 45 (C1) | 10 | 0 | 43.5 | 1.5 | 81.0 | 0.062 | 91.7 |
| SM (lot 2) | | | | | | | | |
| Formulation-4-SM | 45 (C1) | 15 | 0 | 38.5 | 1.5 | 94.7 | 0.002 | 89.8 |
| Formulation-5-SM | 35 (C1) | 30 | 0 | 33.5 | 1.5 | 90.1 | 0.069 | 90.3 |

TABLE 6.2-3
Physical characterization of LNP containing different cationic lipids.

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-1- | 45 (C1) | 0 | 10 | 43.5 | 1.5 | 70.2 | 0.038 | 93.1 |
| Control (lot 2) | | | | | | | | |
| Formulation-1- | 45 (C1) | 10 | 0 | 43.5 | 1.5 | 81.0 | 0.062 | 91.7 |
| SM (lot 2) | | | | | | | | |
| Formulation-7- | 50 (Lipid 5) | 0 | 10 | 38.5 | 1.5 | 73.1 | 0.062 | 93.7 |
| Control | | | | | | | | |
| Formulation-7-SM | 50 (Lipid 5) | 10 | 0 | 38.5 | 1.5 | 89.6 | 0.096 | 89.8 |

As shown in Tables 6.2-1 to 6.2-3, swapping DSPC with sphingomyelin at the same molar percentage in the LNP formulations did not significantly impact the encapsulation efficiency, but slightly increased the average particle size of the final LNP composition. As shown in Table 3, two different ionizable lipids (C₁ and Lipid 5, below) were used together with Sphingomyelin or DSPC to form the LNP formulations.

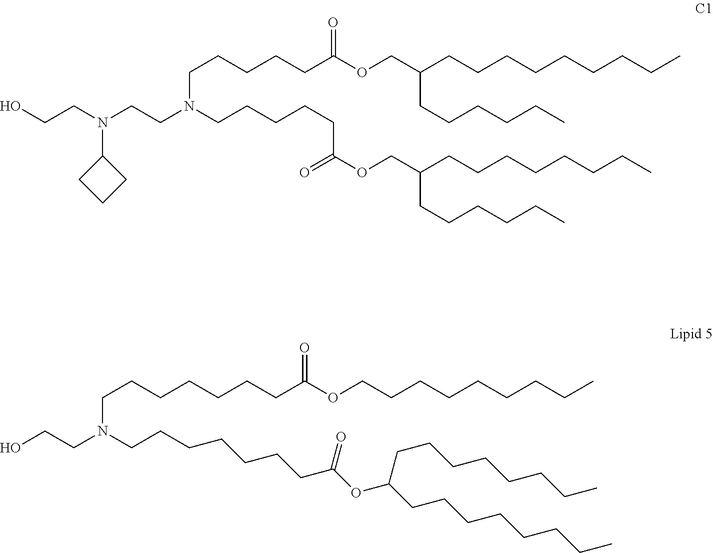 [SMILES: CCCCCCCCCC(CCCCCC)COC(=O)CCCCCN(CCCCCC(=O)OCC(CCCCCC)CCCCCCCC)CCN(CCO)C1CCC1 ; CCCCCCCCCOC(=O)CCCCCCCN(CCO)CCCCCCCC(=O)OC(CCCCCCCC)CCCCCCCC]

### 6.3 Example 3: Microscopic Structure of Lipid Nanoparticles

Five microliters of sample were applied onto a glow-discharged, 400 mesh Cu grid with thin carbon film supported by lacey carbon substrate (Ted Pella). Grids were blotted for 3s and then plunged into liquid nitrogen using a Vitribot Mark IV (FEI). Movie stacks were recorded with a K2 Summit camera (Gatan) in counting mode with a magnification of ×36,000 on a Talos Arctica microscope (FEI) operated at 200 kV.

FIGS. 2B and 2C are Cryo-EM images showing the morphology of the present LRNP (Formulation-1-SM) and a reference LNP (Formulation-1-Control) that were assessed and characterized using the Cryo-EM analysis described herein. FIG. 2B shows at least 56% particles in the present LRNP formulation assumed the semi-lamellar morphology. Particularly, Arrow A points to the lamellar structure and Arrow B points to the non-lamellar electron-dense structure. Scale bar 200 nm. In contrast, FIG. 2C shows that at least 99.9% particles in the reference formulation assumed electron-dense morphology having the solid dark appearance in the whole particles. The observed difference in particle morphology could be attributed to the formation of sphingomyelin-rich rafts in the present LRNP formulation.

FIG. 5 shows Cryo-EM images showing the morphology of LNP formulations containing sphingomyelin at a molar percentage of 10% (Formulation-1-SM), 15% (Formulation-4-SM) and 30% (Formulation-5-SM), respectively. As shown, for the formulations having 10% or 15% sphingomyelin, the majority of nanoparticles in the sampled field of view assumed the semi-lamellar morphology, consistent with FIG. 2B. In contrast, when the sphingomyelin content was increased to 30%, the majority of nanoparticles in the sampled field of view transitioned into a fully lamellar morphology, with a lamellar layer formed surrounding an electron-dense core (e.g., the arrow-pointed particles having the shell-and-core shapes).

### 6.4 Example 4: In Vitro Protein Expression

Lipid nanoparticles encapsulating green fluorescent protein (GFP) mRNA were prepared as described above. Hela cell line was seeded in a 96-well plate. LNP formulation was mixed with 10 μg/mL ApoE at 1:1 (v/v) ratio, then added to the cells at 200 ng/well or 400 ng/well concentrations, and incubated for 36 to 48 hours. GFP intensity was measured with Promega® CellTiter-Glo® luminescent cell viability assay following manufacturer's instructions, and fluorescence intensity (relative light units; RLU) of different groups were plotted in FIG. 3A to 3C, showing the mean value and standard deviation (SD) of at least five repeated experiments for each group.

As shown in FIGS. 3A to 3C, in vitro expression of mRNA contained in a LNP composition varied with the LNP formulation. Particularly, FIG. 3A shows that GFP expression in Hela cells was significantly increased (by 2-folds) when the mRNA was formulated in LNP composition containing sphingomyelin (Formulation-1-SM), as compared to a reference LNP composition that contained the same molar percentage of DSPC instead of sphingomyelin (Formulation-1-Control).

Furthermore, changing sphingomyelin content in a LNP composition significantly affects in vitro expression of mRNA contained in the LNP. Particularly, FIG. 3A shows that reducing sphingomyelin content from 10 mol % (Formulation-1-SM) to 5 mol % (Formulation-3-SM) reduced GFP expression by half FIG. 3B shows that increasing sphingomyelin content from 10 mol % (Formulation-1-SM) to 15 mol % (Formulation-4-SM) significantly enhanced GFP expression by folds.

FIG. 3C shows that enhanced in vitro GFP expression were similarly observed from LNP compositions formulated using sphingomyelin in combination with different types of cationic lipids, including C₁ and Lipid 5.

### 6.5 Example 5: In Vivo Protein Expression

Lipid nanoparticles encapsulating human erythropoietin (hEPO) mRNA were prepared as described above, and systemically administered to 6-8 week old female ICR mice (Xipuer-Bikai, Shanghai) at 0.5 mg/kg dose by tail vein injection. Mice were euthanized by CO₂ overdoses at 6 hours post administration, and blood samples were taken for hEPO measurement. Particularly, serum were separated from total blood by centrifugation at 5000 g for 10 minutes at 4° C., snap-frozen and stored at −80° C. for analysis. The serum hEPO level was measured using an ELSA assay carried out using a commercial kit (DEP00, R&D systems) according to manufacturer's instructions. The hEPO expression levels (μg/ml) measured from the tested group are plotted in FIG. 4, showing the mean value and standard deviation (SD) of at least five repeated experiments (test animals) for each group.

As shown, in vivo expression of mRNA contained in a LNP composition varied with the LNP formulation. Particularly, in vivo mRNA expression was significantly increased (by 1.5-fold) when the mRNA was formulated in LNP composition containing sphingomyelin (Formulation-1-SM), as compared to a reference LNP composition that contained the same molar percentage of DSPC instead of sphingomyelin (Formulation-1-Control).

### 6.6 Example 6: Characterization of LNP Formulations Having Varied Sphingomyelin Contents

The following studies were performed to examine possible impact of the sphingomyelin content in a LNP formulation on the expression level of the nucleic acid molecule in the LNP formulation.

Particularly, LNP formulations containing between 0 and 35% sphingomyelin and human erythropoietin (hEPO) mRNA were prepared as described in Example 1. Physical properties of the nanoparticles were measured for the final formulation to ensure quality of the LNP preparations.

TABLE 6.6
LNP formulations containing different sphingomyelin contents.

| | | | Size | | |
|---|---|---|---|---|---|
| Formulation ID | Composition | Mol % | (nm) | PDI | EE % |
| Formulation-3-SM | C1/SM/Chol/DMG-PEG | 45/5/48.5/1.5 | 82.37 | 0.085 | 91.2 |
| Formulation-1-SM | C1/SM/Chol/DMG-PEG | 45/10/43.5/1.5 | 84.54 | 0.08 | 90.3 |
| Formulation-4-SM | C1/SM/Chol/DMG-PEG | 45/15/38.5/1.5 | 90.41 | 0.01 | 88.1 |
| Formulation-17-SM | C1/SM/Chol/DMG-PEG | 45/20/33.5/1.5 | 91.87 | 0.01 | 84.8 |
| Formulation-19-SM | C1/SM/Chol/DMG-PEG | 45/30/23.5/1.5 | 81.12 | 0.016 | 87.2 |
| Formulation-20-SM | C1/SM/Chol/DMG-PEG | 45/35/18.5/1.5 | 80.51 | 0.074 | 86.9 |
| Formulation-21-SM | C1/SM/Chol/DMG-PEG | 45/40/13.5/1.5 | 74.51 | 0.079 | 85.7 |
| Formulation-22-SM | C1/SM/Chol/DMG-PEG | 45/45/8.5/1.5 | 61.28 | 0.048 | 89 |
| Formulation-23-SM | C1/SM/Chol/DMG-PEG | 40/15/43.5/1.5 | 94.45 | 0.054 | 92.5 |
| Formulation-24-SM | C1/SM/Chol/DMG-PEG | 40/20/38.5/1.5 | 94.39 | 0.056 | 90.9 |
| Formulation-1-Control | C1/DSPC/Chol/DMG-PEG | 45/10/43.5/1.5 | 78.32 | 0.052 | 92.3 |

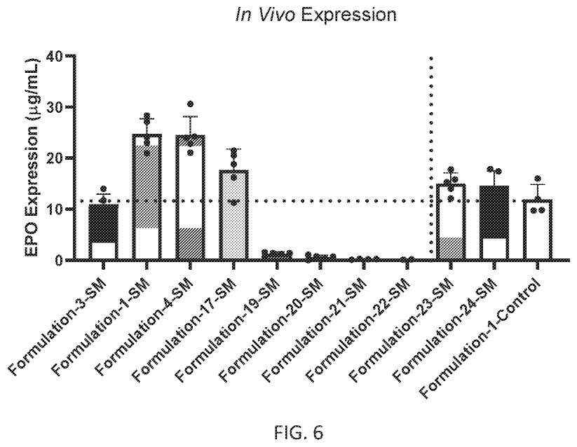

Each LNP formulation was systemically administered to 6-8 week old female ICR mice (Xipuer-Bikai, Shanghai) at 0.5 mg/kg dose by tail vein injection, and blood samples were taken for hEPO measurement as described herein. The hEPO expression levels (μg/ml) measured from the tested group are plotted in FIG. 6, showing the mean value and standard deviation (SD) of at least five repeated experiments (test animals) for each group.

As shown in FIG. 6, in vivo expression of mRNA contained in a LNP composition varied with the molar percentage of sphingomyelin. LNPs comprising 10% sphingomyelin (Formulation-1-SM) or 15% sphingomyelin (Formulation-4-SM) produced the highest protein expression levels in mice and also outperformed the DSPC control. Reducing the molar percentage of sphingomyelin from 10% to 5% significantly reduced the EPO expression level. Increasing the molar percentage of sphingomyelin from 20% to 25% or above also significantly reduced the EPO expression level. Further increasing the sphingomyelin molar percentage to above 30% resulted in minimal EPO expression in vivo. FIG. 6 also shows that the molar percentage of cationic lipid (C₁) affected the protein expression level, with LNP comprising 45% of C₁ (Formulation-4-SM) produced higher protein expression level than LNP comprising 40% of C₁ (Formulation-23-SM).

### 6.7 Example 7: Characterization of LNP Formulations Having Varied Sphingomyelin Molecules

To further investigate possible impacts of the sphingomyelin tail length on the LNP formulations as described herein, sphingomyelin molecules having the amide-linked acyl chains of different lengths (e.g., from 12 to 24 carbons) (Table X) were used to formulate LNP formulations, and the LNP formulations were further characterized as described below.

TABLE X
Exemplary Sphingomyelin Compounds.

| Abbreviation | Sphingomyelin | Molecule Structure |
|---|---|---|
| SM-01 | 12:0(d18:1/12:0) | 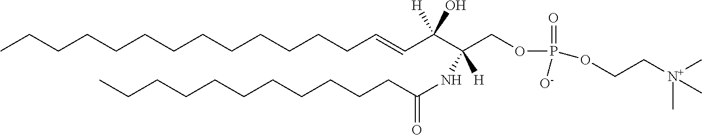 [SMILES: CCCCCCCCCCCCC/C=C/[C@@H](O)[C@H](COP(=O)([O-])OCC[N+](C)(C)C)NC(=O)CCCCCCCCCCC] |
| SM-02 | 14:0(d18:1/14:0) | 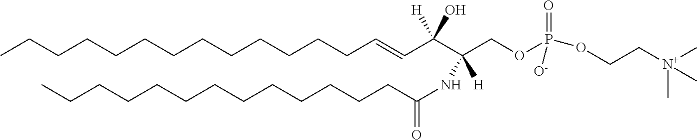 [SMILES: CCCCCCCCCCCCC/C=C/[C@@H](O)[C@H](COP(=O)([O-])OCC[N+](C)(C)C)NC(=O)CCCCCCCCCCCCC] |
| SM-03 (or SM) | 16:0(d18:1/16:0) | 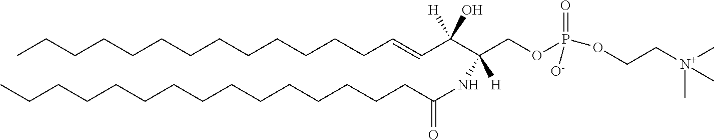 [SMILES: CCCCCCCCCCCCC/C=C/[C@@H](O)[C@H](COP(=O)([O-])OCC[N+](C)(C)C)NC(=O)CCCCCCCCCCCCCCC] |
| SM-04 | 16:1(d18:1/16:1(9Z)) | 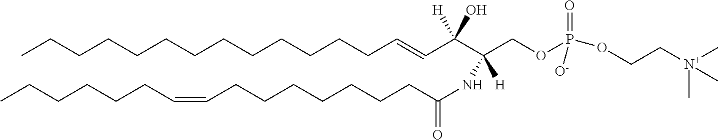 [SMILES: CCCCCC/C=C\CCCCCCCC(=O)N[C@@H](COP(=O)([O-])OCC[N+](C)(C)C)[C@H](O)/C=C/CCCCCCCCCCCCC] |
| SM-05 | 18:1(d18:1/18:1(9Z)) | 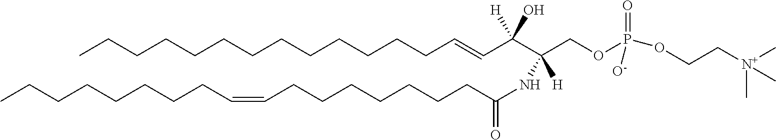 [SMILES: CCCCCCCC/C=C\CCCCCCCC(=O)N[C@@H](COP(=O)([O-])OCC[N+](C)(C)C)[C@H](O)/C=C/CCCCCCCCCCCCC] |
| SM-06 | 24:0(d18:1/24:0) | 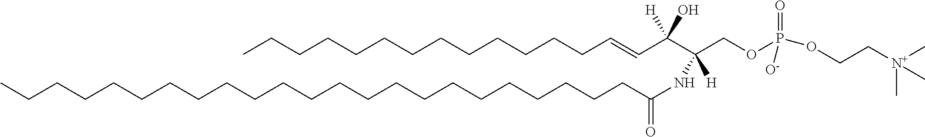 [SMILES: CCCCCCCCCCCCC/C=C/[C@@H](O)[C@H](COP(=O)([O-])OCC[N+](C)(C)C)NC(=O)CCCCCCCCCCCCCCCCCCCCCCC] |
| SM-07 | 18:0 (d18:1/18:0) | 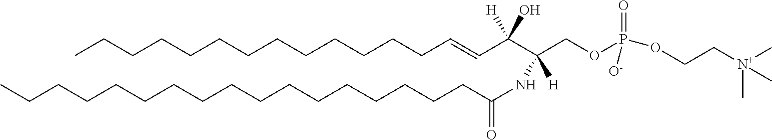 [SMILES: CCCCCCCCCCCCC/C=C/[C@@H](O)[C@H](COP(=O)([O-])OCC[N+](C)(C)C)NC(=O)CCCCCCCCCCCCCCCCC] |

Particularly, lipid nanoparticles containing human erythropoietin (hEPO) mRNA were prepared as described in Example 1. Physical properties of the nanoparticles (size, PDI, EE %) were measured for the final formulation to ensure quality of the LNP preparations, and summarized in Table 6.7.

TABLE 6.7
LNP Formulations having Sphingomyelin of different tail
lengths and exemplary (lot-specific) physical properties.

| | Mol % | | | | Final LNP | | |
|---|---|---|---|---|---|---|---|
| | Cationic | Sphingo- | Choles- | DMG- | Size | | |
| Formulation ID | Lipid | myelin | terol | PEG | (nm) | PDI | EE % |
| Formulation-1-SM01 | 45 (C1) | 10 (SM-01) | 43.5 | 1.5 | 81.57 | 0.059 | 92.0% |
| Formulation-1-SM02 | 45 (C1) | 10 (SM-02) | 43.5 | 1.5 | 83.88 | 0.068 | 87.8% |
| Formulation-1-SM | 45 (C1) | 10 (SM-03) | 43.5 | 1.5 | 95.25 | 0.043 | 91.2% |
| Formulation-1-SM04 | 45 (C1) | 10 (SM-04) | 43.5 | 1.5 | 84.02 | 0.099 | 90.8% |
| Formulation-1-SM05 | 45 (C1) | 10 (SM-05) | 43.5 | 1.5 | 80.56 | 0.068 | 91.9% |

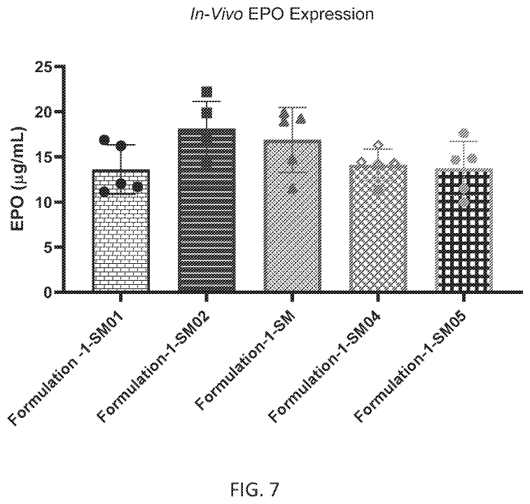

Each LNP formulation was systemically administered to 6-8 week old female ICR mice (Xipuer-Bikai, Shanghai) at 0.5 mg/kg dose by tail vein injection, and blood samples were taken for hEPO measurement as described herein. The hEPO expression levels (μg/ml) measured from the tested group are plotted in FIG. 7, showing the mean value and standard deviation (SD) of at least five repeated experiments (test animals) for each group. As shown, the sphingomyelins tail length did not impact the in vivo mRNA expression level in a statistically significant manner. Particularly, all five LNP formulations tested in this study produced high protein expression levels, with the LNP formulations comprising SM-03 and SM-02 produced the highest protein expression level.

### 6.8 Example 8: Characterization of LNP Formulations Having Varied Cationic Lipids

To investigate possible impacts of cationic lipids on sphingomyelin-containing LNP formulations described herein, cationic lipids having diversified structures as listed in Table Y below were each used to formulate LNP formulations either with sphingomyelin (SM-03) or with equivalent amounts of DSPC (as control), and the formulations were further characterized as described below.

TABLE Y
Cationic Lipids.

| | |
|---|---|
| C1 | 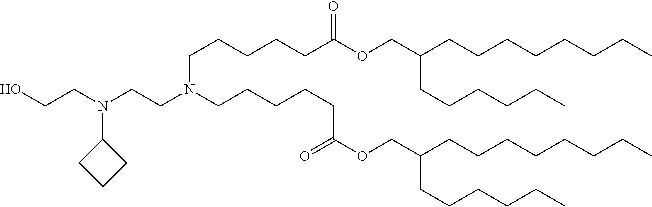 [SMILES: CCCCCCCCC(CCCCCC)COC(=O)CCCCCN(CCCCCC(=O)OCC(CCCCCC)CCCCCCCC)CCN(CCO)C1CCC1] |
| C2 | 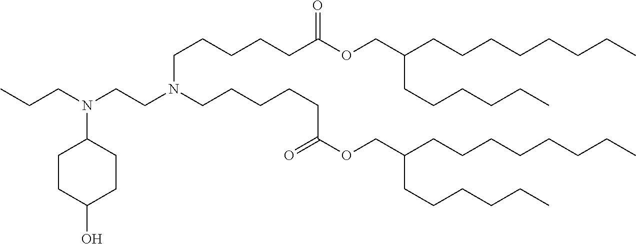 [SMILES: CCCCCCCCC(CCCCCC)COC(=O)CCCCCN(CCCCCC(=O)OCC(CCCCCC)CCCCCCCC)CCN(CCC)C1CCC(O)CC1] |
| C3 | 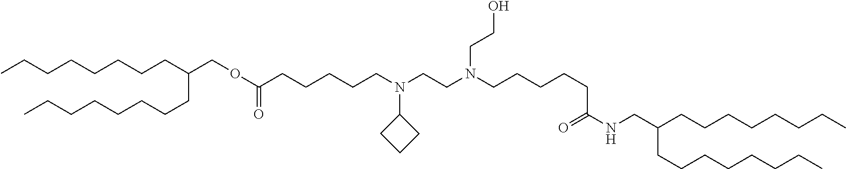 [SMILES: CCCCCCCCC(CCCCCCCC)CNC(=O)CCCCCN(CCO)CCN(CCCCCC(=O)OCC(CCCCCCCC)CCCCCCCC)C1CCC1] |
| C4 | 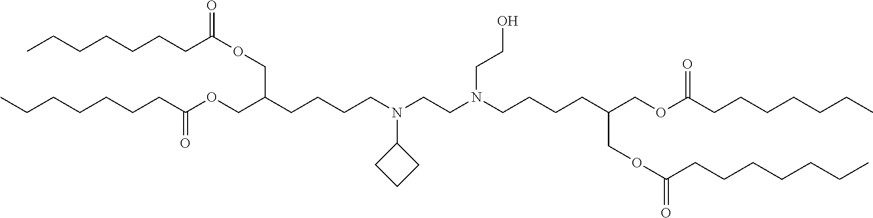 [SMILES: CCCCCCCC(=O)OCC(CCCCN(CCO)CCN(CCCCC(COC(=O)CCCCCCC)COC(=O)CCCCCCC)C1CCC1)COC(=O)CCCCCCC] |
| C5 | 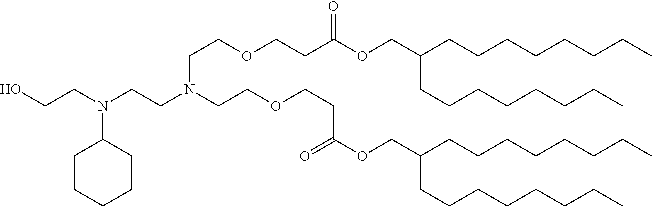 [SMILES: CCCCCCCCC(CCCCCCCC)COC(=O)CCOCCN(CCOCCC(=O)OCC(CCCCCCCC)CCCCCCCC)CCN(CCO)C1CCCCC1] |
| C6 | 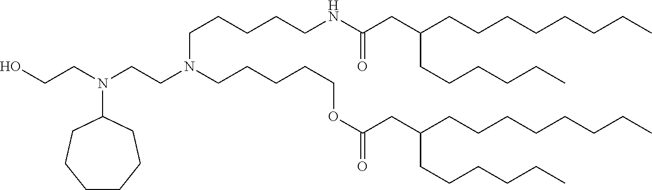 [SMILES: CCCCCCCCC(CCCCCC)CC(=O)NCCCCCN(CCCCCOC(=O)CC(CCCCCC)CCCCCCCC)CCN(CCO)C1CCCCCC1] |
| C7 | 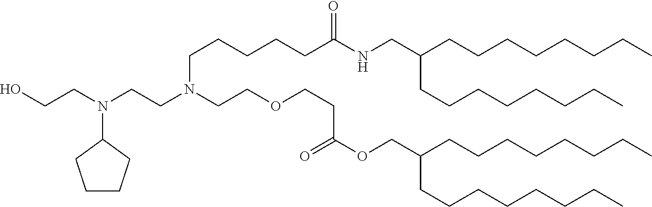 [SMILES: CCCCCCCCC(CCCCCCCC)CNC(=O)CCCCCN(CCOCCC(=O)OCC(CCCCCCCC)CCCCCCCC)CCN(CCO)C1CCCC1] |
| C8 | 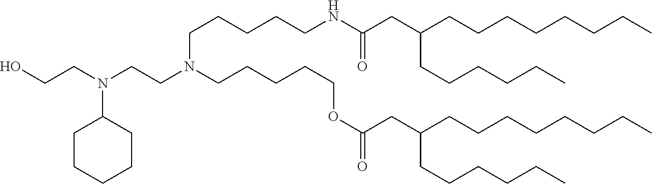 [SMILES: CCCCCCCCC(CCCCCC)CC(=O)NCCCCCN(CCCCCOC(=O)CC(CCCCCC)CCCCCCCC)CCN(CCO)C1CCCCC1] |
| C9 | 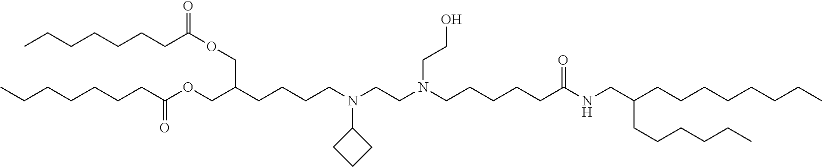 [SMILES: CCCCCCCCC(CCCCCC)CNC(=O)CCCCCN(CCO)CCN(CCCCC(COC(=O)CCCCCCC)COC(=O)CCCCCCC)C1CCC1] |
| C10 | 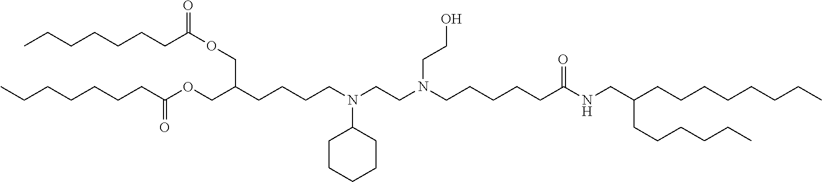 [SMILES: CCCCCCCCC(CCCCCC)CNC(=O)CCCCCN(CCO)CCN(CCCCC(COC(=O)CCCCCCC)COC(=O)CCCCCCC)C1CCCCC1] |
| C11 | 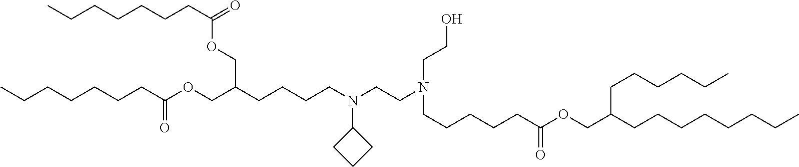 [SMILES: CCCCCCCCC(CCCCCC)COC(=O)CCCCCN(CCO)CCN(CCCCC(COC(=O)CCCCCCC)COC(=O)CCCCCCC)C1CCC1] |
| C12 | 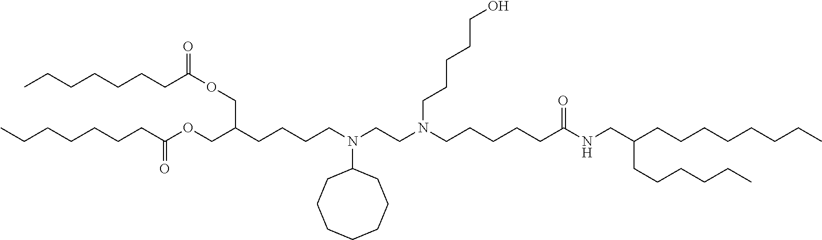 [SMILES: CCCCCCCCC(CCCCCC)CNC(=O)CCCCCN(CCCCCO)CCN(CCCCC(COC(=O)CCCCCCC)COC(=O)CCCCCCC)C1CCCCCCC1] |
| C13 | 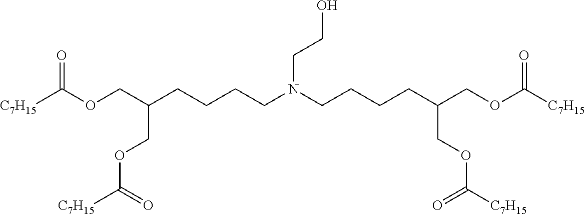 |
| C14 | 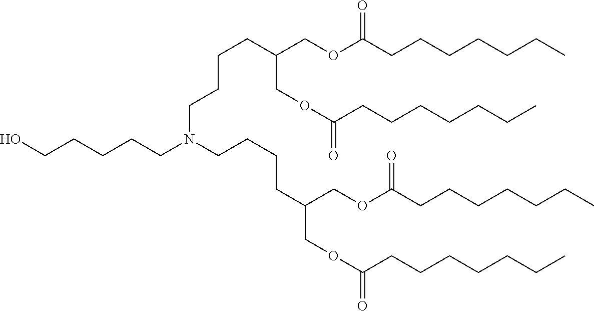 [SMILES: CCCCCCCC(=O)OCC(CCCCN(CCCCCO)CCCCC(COC(=O)CCCCCCC)COC(=O)CCCCCCC)COC(=O)CCCCCCC] |
| C15 | 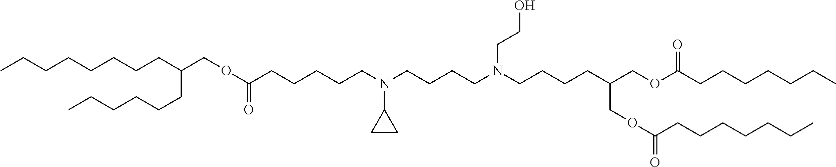 [SMILES: CCCCCCCCC(CCCCCC)COC(=O)CCCCCN(CCCCN(CCO)CCCCC(COC(=O)CCCCCCC)COC(=O)CCCCCCC)C1CC1] |
| C16 | 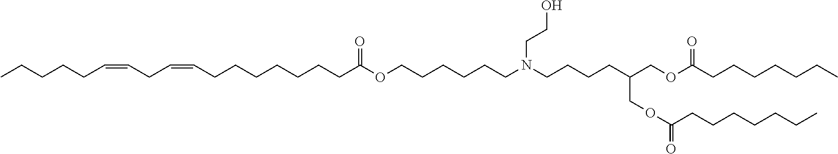 [SMILES: CCCCC/C=C\C/C=C\CCCCCCCC(=O)OCCCCCCN(CCO)CCCCC(COC(=O)CCCCCCC)COC(=O)CCCCCCC] |
| C17 | 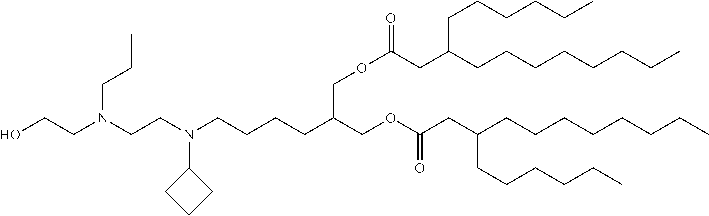 [SMILES: CCCCCCCCC(CCCCCC)CC(=O)OCC(CCCCN(CCN(CCC)CCO)C1CCC1)COC(=O)CC(CCCCCC)CCCCCCCC] |
| SM- 102 | 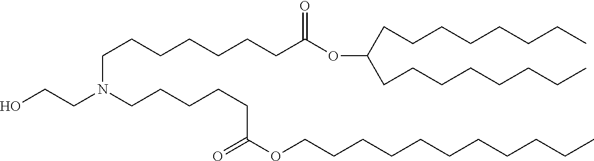 [SMILES: CCCCCCCCCCCOC(=O)CCCCCN(CCO)CCCCCCCC(=O)OC(CCCCCCCC)CCCCCCCC] |
| ALC- 0315 | 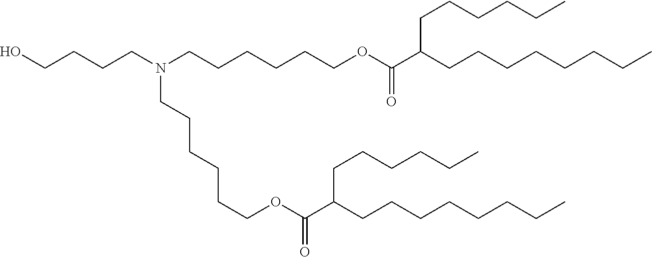 [SMILES: CCCCCCCCC(CCCCCC)C(=O)OCCCCCCN(CCCCO)CCCCCCOC(=O)C(CCCCCC)CCCCCCCC] |
| Lipid- 5 | 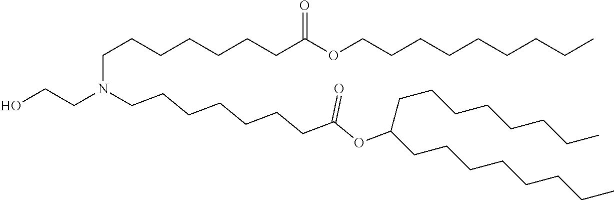 [SMILES: CCCCCCCCCOC(=O)CCCCCCCN(CCO)CCCCCCCC(=O)OC(CCCCCCCC)CCCCCCCC] |

### 6.8.1 Study No: 1—In Vitro GFP Expression

In this study, lipid nanoparticles containing green fluorescent protein (GFP) encoding mRNA were prepared as described in Example 1, and physical properties of the nanoparticles in the final formulation were assessed to ensure quality of the LNP formulations, as summarized in Table 6.8.1.

TABLE 6.8.1
LNP Formulations having different cationic lipids
and exemplary (lot-specific) physical properties

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-8-Control | 50 (C2) | 0 | 10 | 38.5 | 1.5 | 90.4 | 0.032 | 94.9% |
| Formulation-8-SM | 50 (C2) | 10 | 0 | 38.5 | 1.5 | 105.3 | 0.025 | 92.9% |
| Formulation-10-Control | 50 (C3) | 0 | 10 | 38.5 | 1.5 | 68.0 | 0.012 | 93.2% |
| Formulation-10-SM | 50 (C3) | 10 | 0 | 38.5 | 1.5 | 89.5 | 0.024 | 91.8% |

Hela cell line was seeded in a 96-well plate. LNP formulation was mixed with 10 μg/mL ApoE at 1:1 (v/v) ratio at 37° C. for 15 min, then added to the cells at 400 ng/well concentrations, and incubated for 36 to 48 hours. GFP intensity was measured with Promega® CellTiter-Glo® luminescent cell viability assay following manufacturer's instructions, and fluorescence intensity (relative light units; RLU) of different groups were plotted in FIG. 8, showing the mean value and standard deviation (SD) of at least three repeated experiments for each group.

As shown in FIG. 8, for both tested cationic lipids, the average in vitro expression level of mRNA was higher from the sphingomyelin containing composition as compared to the corresponding control formulation containing an equivalent amount of DSPC instead of sphingomyelin. Particularly, for LNP formulations containing cationic lipid C₃, the mRNA expression from sphingomyelin-containing LNP was significantly higher than mRNA expression from the corresponding DSPC control formulation.

### 6.8.2 Study No.: 2—In Vivo EPO Expression

In this study, lipid nanoparticles containing human erythropoietin (hEPO) mRNA were prepared as described in Example 1. Physical properties of the nanoparticles in the final formulation were assessed to ensure qualify of the LNP preparations, as summarized in Table 6.8.2.

TABLE 6.8.2
LNP Formulations having different cationic lipids and exemplary (lot-specific) physical properties.

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-1B-Control | 50 (C1) | 0 | 10 | 38.5 | 1.5 | 61.2 | 0.05 | 90.7% |
| Formulation-1B-SM | 50 (C1) | 10 (SM) | 0 | 38.5 | 1.5 | 85.2 | 0.056 | 90.3% |
| Formulation-11-Control | 50 (C4) | 0 | 10 | 38.5 | 1.5 | 84.6 | 0.09 | 82.7% |
| Formulation-11-SM | 50 (C4) | 10 (SM) | 0 | 38.5 | 1.5 | 112.2 | 0.08 | 73.4% |
| Formulation-12-Control | 50 (C5) | 0 | 10 | 38.5 | 1.5 | 81.3 | 0.04 | 95.4% |
| Formulation-12-SM | 50 (C5) | 10 (SM) | 0 | 38.5 | 1.5 | 108.8 | 0.03 | 93.7% |
| Formulation-13-Control | 50 (C6) | 0 | 10 | 38.5 | 1.5 | 80.7 | 0.07 | 82.2% |
| Formulation-13-SM | 50 (C6) | 10 (SM) | 0 | 38.5 | 1.5 | 82.8 | 0.05 | 84.8% |
| Formulation-15-Control | 50 (C7) | 0 | 10 | 38.5 | 1.5 | 67.7 | 0.04 | 91.6% |
| Formulation-15-SM | 50 (C7) | 10 (SM) | 0 | 38.5 | 1.5 | 95.4 | 0.03 | 85.3% |
| Formulation-16-Control | 50 (C8) | 0 | 10 | 38.5 | 1.5 | 70.5 | 0.06 | 88.7% |
| Formulation-16-SM | 50 (C8) | 10 (SM) | 0 | 38.5 | 1.5 | 93.0 | 0.04 | 81.3% |
| Formulation-27-Control | 50 (C9) | 0 | 10 | 38.5 | 1.5 | 81.0 | 0.076 | 89.7% |
| Formulation-27-SM | 50 (C9) | 10 (SM) | 0 | 38.5 | 1.5 | 116.8 | 0.01 | 82.0% |
| Formulation-28-Control | 50 (C10) | 0 | 10 | 38.5 | 1.5 | 66.9 | 0.054 | 89.4% |
| Formulation-28-SM | 50 (C10) | 10 (SM) | 0 | 38.5 | 1.5 | 97.4 | 0.01 | 83.2% |
| Formulation-29-Control | 50 (C11) | 0 | 10 | 38.5 | 1.5 | 68.6 | 0.021 | 88.4% |
| Formulation-29-SM | 50 (C11) | 10 (SM) | 0 | 38.5 | 1.5 | 96.3 | 0.02 | 84.0% |
| Formulation-30-Control | 50 (C12) | 0 | 10 | 38.5 | 1.5 | 69.5 | 0.017 | 87.2% |
| Formulation-30-SM | 50 (C12) | 10 (SM) | 0 | 38.5 | 1.5 | 68.2 | 0.074 | 89.1% |
| Formulation-31-Control | 50 (C13) | 0 | 10 | 38.5 | 1.5 | 61.3 | 0.09 | 91.5% |
| Formulation-31-SM | 50 (C13) | 10 (SM) | 0 | 38.5 | 1.5 | 78.0 | 0.05 | 86.9% |
| Formulation-32-Control | 50 (C14) | 0 | 10 | 38.5 | 1.5 | 81.5 | 0.07 | 86.7% |
| Formulation-32-SM | 50 (C14) | 10 (SM) | 0 | 38.5 | 1.5 | 102.9 | 0.11 | 76.9% |
| Formulation-33-Control | 50 (C15) | 0 | 10 | 38.5 | 1.5 | 64.8 | 0.09 | 92.6% |
| Formulation-33-SM | 50 (C15) | 10 (SM) | 0 | 38.5 | 1.5 | 87.8 | 0.12 | 87.4% |
| Formulation-34-Control | 50 (C16) | 0 | 10 | 38.5 | 1.5 | 69.8 | 0.11 | 91.0% |
| Formulation-34-SM | 50 (C16) | 10 (SM) | 0 | 38.5 | 1.5 | 91.1 | 0.14 | 87.4% |
| Formulation-35-Control | 50 (C17) | 0 | 10 | 38.5 | 1.5 | 73.4 | 0.03 | 90.8% |
| Formulation-35-SM | 50 (C17) | 10 (SM) | 0 | 38.5 | 1.5 | 90.4 | 0.05 | 92.2% |
| Formulation-36-Control | 50 (SM-102) | 0 | 10 | 38.5 | 1.5 | 60.7 | 0.05 | 92.5% |
| Formulation-36-SM | 50 (SM-102) | 10 (SM) | 0 | 38.5 | 1.5 | 72.8 | 0.01 | 91.8% |
| Formulation-37-Control | 50 (ALC-0315) | 0 | 10 | 38.5 | 1.5 | 61.5 | 0.08 | 86.2% |
| Formulation-37-SM | 50 (ALC-0315) | 10 (SM) | 0 | 38.5 | 1.5 | 82.2 | 0.05 | 83.8% |

As shown in Table 6.8.2, all lipid nanoparticles containing different cationic lipids in combination with either sphingomyelin or an equivalent amount of DSPC (as a corresponding control) had particle sizes, PDI and encapsulation efficiencies within the expected ranges.

Each LNP formulation was systemically administered to 6-8 week old female ICR mice (Xipuer-Bikai, Shanghai) at 0.5 mg/kg dose by tail vein injection, and blood samples were taken for hEPO measurement as described herein. The hEPO expression levels (μg/ml) measured from the tested group are plotted in FIG. 9, showing the mean value and standard deviation (SD) of at least five repeated experiments (test animals) for each group. As shown, for each of the tested cationic lipids, the average in vivo expression level of mRNA was higher for the sphingomyelin containing composition as compared to the corresponding control formulation containing an equivalent amount of DSPC instead of sphingomyelin. Particularly, for LNP formulations containing cationic lipids C1, C5 to C9, C14, C16, and ALC-0315, mRNA expression from sphingomyelin-containing LNP was significantly higher than mRNA expression from the corresponding DSPC control formulation.

### 6.9 Example 9: Tissue-Specific Expression of Nucleic Acid Molecules Delivered in LNP Formulations

To study the tissue biodistribution of LNPs in mice, LNP formulations listed in Table 6.9 containing mRNA encoding luciferase were prepared as described in Example 1. Physical properties of the nanoparticles in the final formulations were assessed to ensure quality of the LNP preparations, as summarized in Table 6.9.

TABLE 6.9
LNP formulations used for Luciferase mRNA biodistribution
study and exemplary (lot-specific) physical properties.

| | Mol % | | | | | Final LNP | | |
|---|---|---|---|---|---|---|---|---|
| | Cationic | Sphingo- | | Choles- | DMG- | Size | | |
| Formulation ID | Lipid | myelin | DSPC | terol | PEG | (nm) | PDI | EE % |
| Formulation-1B-Control | 50 (C1) | 0 | 10 | 38.5 | 1.5 | 61.2 | 0.05 | 90.7% |
| Formulation-1B-SM | 50 (C1) | 10 (SM) | 0 | 38.5 | 1.5 | 85.2 | 0.056 | 90.3% |
| Formulation-5-SM | 35 (C1) | 30 (SM) | 0 | 33.5 | 1.5 | 90.90 | 0.028 | 94.08% |

As shown in Table 6.9, all lipid nanoparticles containing different cationic lipids in combination with either sphingomyelin or an equivalent amount of DSPC (as a corresponding control) had particle sizes, PDI and encapsulation efficiencies within the expected ranges.

Each formulation was systemically administered to 6-8 week old female ICR mice (Xipuer-Bikai, Shanghai) at 0.5 mg/kg dose by tail vein injection. After 5.75 hours, the mice were subcutaneously administrated with XenoLight D-luciferrin (potassium salt), a substrate of luciferases that catalyze the production of luminescence. The mice were subsequently euthanized by CO₂ overdoses 15 min thereafter. Mice tissues were harvested and placed in a luminescence imaging scanner to measure the expression level of luciferase in each tissue. The fluorescent levels measured from harvest tissues were plotted in FIG. 10, showing the mean value and standard deviation (SD) of at least three repeated experiments (test animals) for each group.

As shown in FIG. 10, in all organs examined, the average luciferase expression level was higher for sphingomyelin containing LNPs (Formulation-1B-SM) as compared to corresponding control formulation containing an equivalent amount of DSPC instead of sphingomyelin (Formulation-1B-control). The observed differences between sphingomyelin containing formulation and the corresponding DSPC control was statistically significant in heart, kidney, liver and lung. The luciferase expression level was highest in liver comparing to other organs examined.

The LNP containing 10% sphingomyelin (Formulation-1B-SM) showed significantly higher luciferase expression level than the LNP containing 30% of Sphingomyelin (Formulation-5-SM) in heart, kidney, liver and lung, indicating sphingomyelin content of 10% molar ratio is more beneficial comparing to 30%.

### 6.10 Example 10: Exemplary Synthesis

General preparative HPLC method: HPLC purification is carried out on a Waters 2767 equipped with a diode array detector (DAD) on an Inertsil Pre-C8 OBD column, generally with water containing 0.1% TFA as solvent A and acetonitrile as solvent B.

General LCMS method: LCMS analysis is conducted on a Shimadzu (LC-MS2020) System. Chromatography is performed on a SunFire C₁₈, generally with water containing 0.1% formic acid as solvent A and acetonitrile containing 0.1% formic acid as solvent B.

6.10.1 Preparation of Compound 02-1 (i.e. Compound 1 in the Following Scheme).

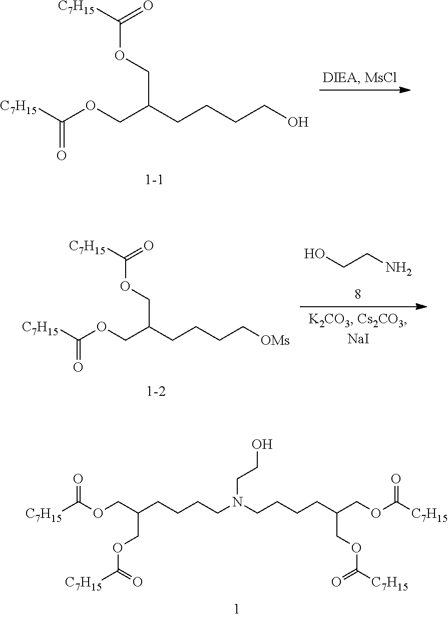

Compound 02-1: ¹H NMR (400 MHz, CDCl₃) δ: 0.86-0.90 (m, 12H), 1.27-1.63 (m, 53H), 1.97-2.01 (m, 2H), 2.28-2.64 (m, 14H), 3.52-3.58 (m, 2H), 4.00-4.10 (m, 8H). LCMS: Rt: 1.080 min: MS m/z (ESI): 826.0 [M+H]⁺.

The following compounds were prepared in analogous fashion as Compound 02-1, using corresponding starting material.

| Compound | Characterization |
|---|---|
| 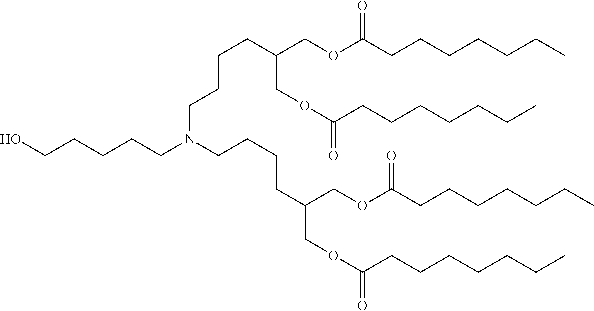 [SMILES: CCCCCCCC(=O)OCC(CCCCN(CCCCCO)CCCCC(COC(=O)CCCCCCC)COC(=O)CCCCCCC)COC(=O)CCCCCCC] | ¹H NMR (400 MHz, CDCl₃) δ: 0.86-0.90 (m, 12H), 1.28-1.32 (m, 30H), 1.35-1.44 (m, 12H), 1.57-1.66 (m, 17H), 1.95-2.01 (m, 2H), 2.28-2.40 (m, 14H), 3.63-3.66 (m, 2H), 4.00-4.11 (m, 8H). LCMS: Rt: 1.140 min; MS m/z (ESI): 868.1 [M + H]⁺. |
| Compound 02-3 | |

6.10.2 Preparation of Compound 02-2 (i.e. Compound 2 in the Following Scheme).

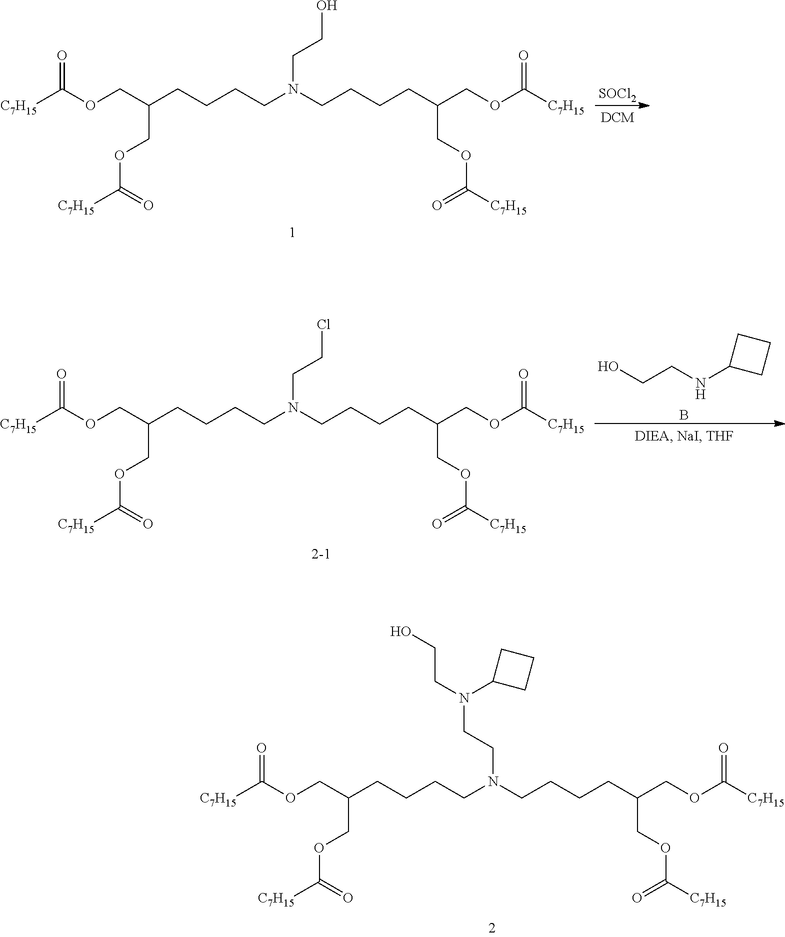

Compound 02-2: ¹H NMR (400 MHz, CDCl₃) δ: 0.86-0.90 (m, 12H), 1.28-1.67 (m, 54H), 1.88-2.01 (m, 7H), 2.28-2.56 (m, 18H), 3.16-3.20 (m, 1H), 3.52-3.54 (m, 2H), 4.00-4.10 (m, 8H). LCMS: Rt: 1.060 min; MS m/z (ESI): 923.0 [M+H]⁺.

6.10.3 Preparation of Compound 02-4 (i.e. Compound 4 in the Following Scheme).

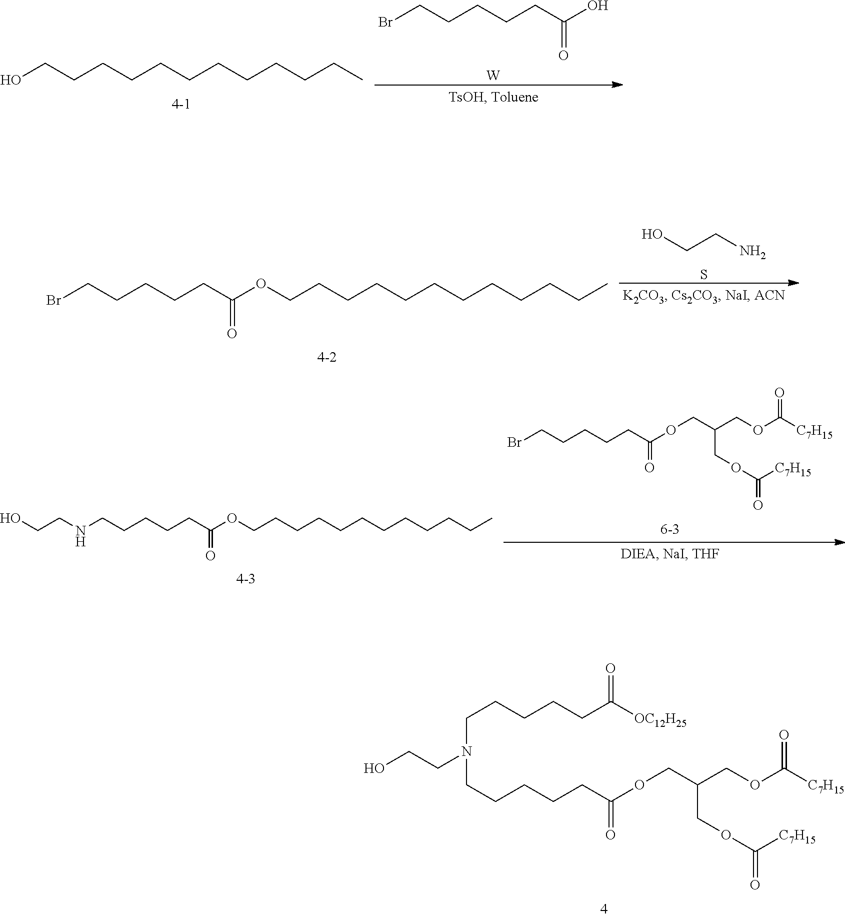

Compound 02-4: ¹H NMR (400 MHz, CDCl₃) δ: 0.86-0.90 (m, 9H), 1.26-1.32 (m, 34H), 1.41-1.49 (m, 4H), 1.61-1.66 (m, 15H), 2.00-2.03 (m, 1H), 2.21-2.38 (m, 8H), 2.43-2.47 (m, 4H), 2.56-2.60 (m, 2H), 3.50-3.54 (m, 2H), 4.03-4.14 (m, 8H). LCMS: Rt: 1.030 min; MS m/z (ESI): 798.0 [M+H]⁺.

6.10.4 Preparation of Compound 02-9 (i.e. Compound 9 in the Following Scheme).

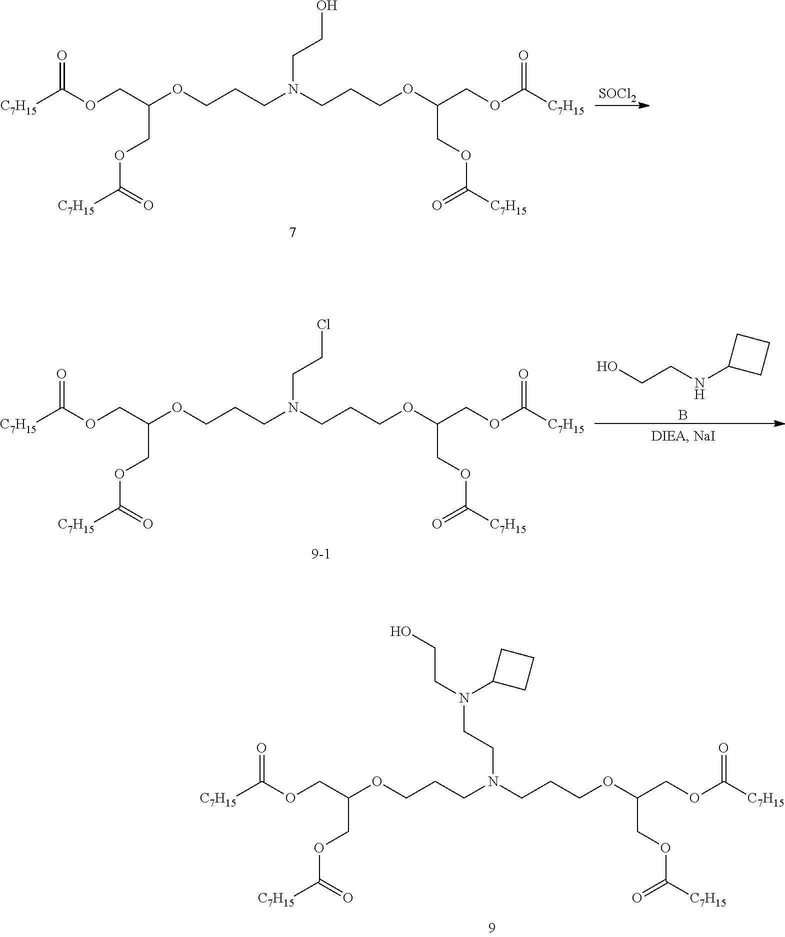

Compound 02-9: ¹H NMR (400 MHz, CDCl₃) δ: 0.86-0.90 (m, 12H), 1.28-1.30 (m, 33H), 1.58-2.01 (m, 18H), 2.30-2.54 (m, 18H), 3.10-3.19 (m, 1H), 3.52-3.68 (m, 8H), 4.09-4.20 (m, 8H). LCMS: Rt: 1.677 min; MS m/z (ESI): 927.7 [M+H]+.

The following compounds were prepared in analogous fashion as Compound 02-9, Compound Characterization

| Compound | Characterization |
|---|---|
| 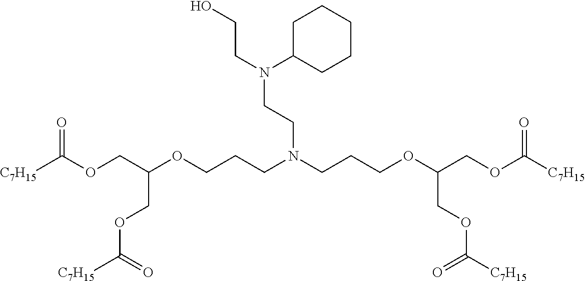 | ¹H NMR (400 MHz, CDCl₃) δ: 0.86-0.90 (m, 12H), 1.28-1.30 (m, 40H), 1.58-1.80 (m, 16H), 2.30-2.60 (m, 18H), 3.58-3.68 (m, 8H), 4.08-4.20 (m, 8H). LCMS: Rt: 1.000 min; MS m/z (ESI): 955.7 [M + H]⁺. |
| Compound 02-14 | |

6.10.5 Preparation of Compound 02-10 (i.e. Compound 10 in the Following Scheme).

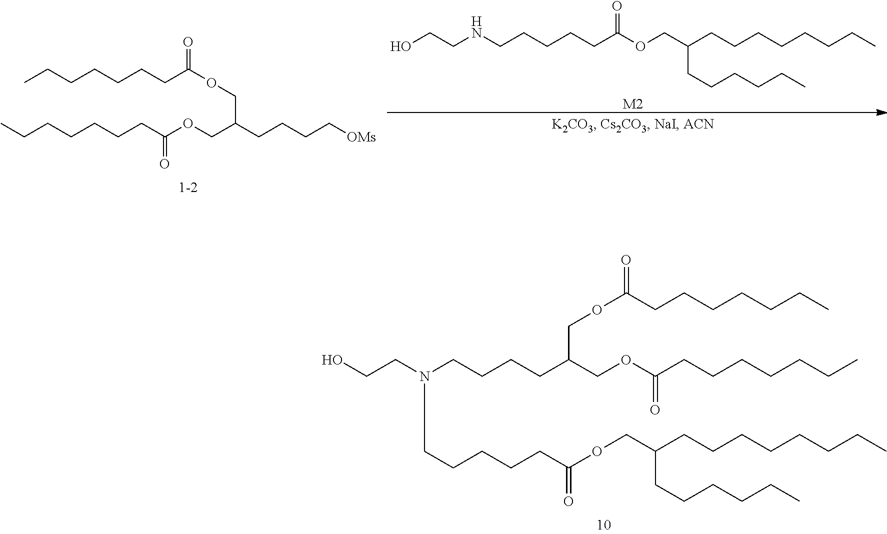 [SMILES: CCCCCCCC(=O)OCC(CCCCOS(C)(=O)=O)COC(=O)CCCCCCC ; CCCCCCCCC(CCCCCC)COC(=O)CCCCCNCCO ; "M2" ; CCCCCCCCC(CCCCCC)COC(=O)CCCCCN(CCO)CCCCC(COC(=O)CCCCCCC)COC(=O)CCCCCCC]

Compound 02-10: ¹H NMR (400 MHz, CDCl₃) δ: 0.86-0.90 (m, 12H), 1.26-1.41 (m, 48H), 1.51-1.72 (m, 11H), 1.94-2.03 (m, 1H), 2.29-2.32 (m, 6H), 2.41-2.91 (m, 5H), 3.51-3.76 (m, 2H), 3.96-4.10 (m, 6H). LCMS: Rt: 1.327 min; MS m/z (ESI): 782.6 [M+H]⁺.

The following compounds were prepared in analogous fashion as Compound 02-10, using corresponding starting material.

| Compound | Characterization |
|---|---|
| 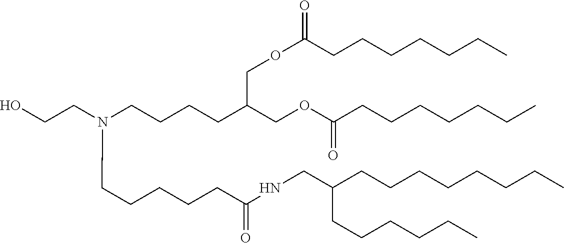 [SMILES: CCCCCCCCC(CCCCCC)CNC(=O)CCCCCN(CCO)CCCCC(COC(=O)CCCCCCC)COC(=O)CCCCCCC] | ¹H NMR (400 MHz, CDCl₃) δ: 0.86-0.90 (m, 12H), 1.26-1.30 (m, 46H), 1.43-1.50 (m, 8H), 1.69-1.73 (m, 3H), 1.83-1.90 (m, 3H), 1.98-2.02 (m, 1H), 2.20-2.33 (m, 6H), 3.05-3.19 (m, 7H), 3.98-4.10 (m, 6H). LCMS: Rt: 1.205 min; MS m/z (ESI): 781.7 [M + H]⁺. |
| Compound 02-11 | |

6.10.6 Preparation of Compound 02-12 (i.e. Compound 12 in the Following Scheme).

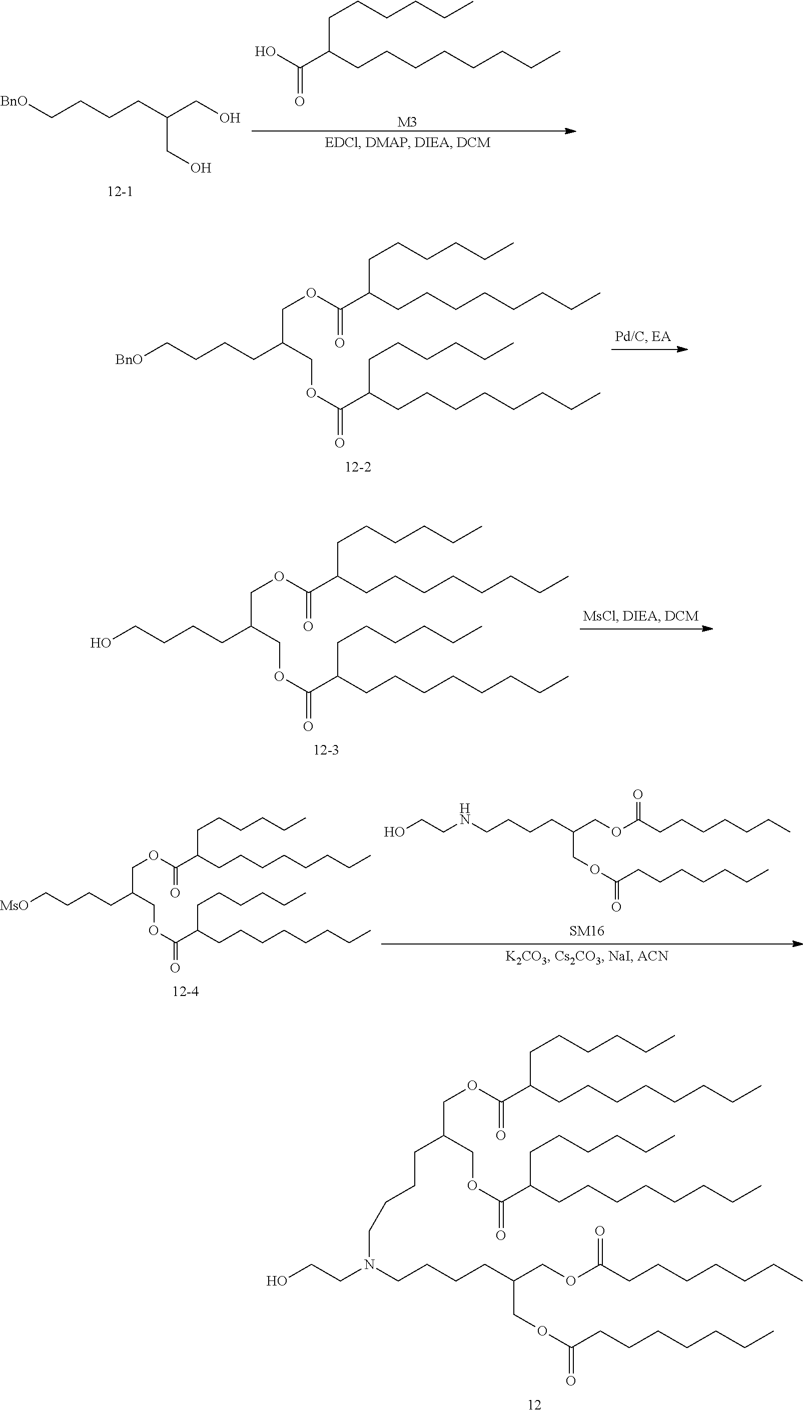

004281 Compound 02-12: ¹H NMR (400 MHz, CDCl₃) δ: 0.86-0.89 (i, 18H), 1.25-1.35 (m, 53H), 1.41-1.48 (m, 8H), 1.56-1.61 (m, 20H), 1.95-2.01 (m, 2H), 2.28-2.35 (m, 6H), 2.43-2.46 (m, 4H), 2.56-2.58 (m, 2H), 3.51-3.54 (m, 2H), 4.00-4.10 (m, 8H). LCMS: Rt: 0.080 min; MS m/z (ESI): 1050.8 [M+H]⁺.

6.10.7 Preparation of Compound 02-20 (i.e. Compound 20 in the Following Scheme).

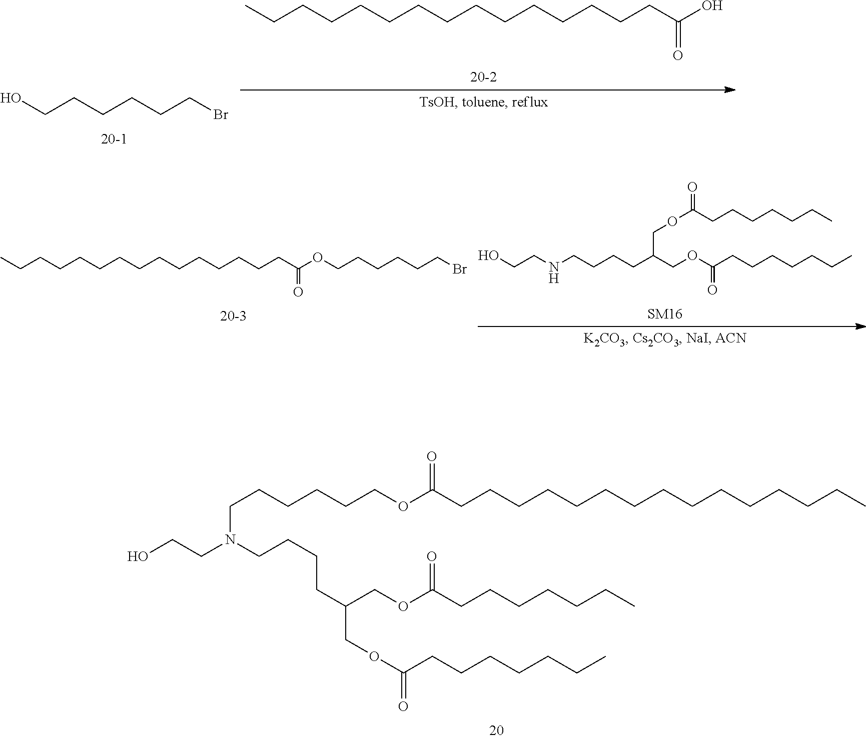

Compound 02-20: ¹H NMR (400 MHz, CDCl3) δ: 0.86-0.90 (m, 9H), 1.25-1.36 (m, 48H), 1.41-1.48 (m, 5H), 1.60-1.62 (m, 8H), 1.97-2.00 (m, 1H), 2.27-2.32 (m, 6H), 2.43-2.46 (m, 4H), 2.56-2.59 (m, 2H), 3.52-3.54 (m, 2H), 4.01-4.10 (m, 6H). LCMS: Rt: 0.093 min; MS m/z (ESI): 782.6 [M+H]⁺.

1. A nanoparticle composition comprising a plurality of lipid nanoparticles, wherein the lipid nanoparticles comprise:
(a) a sphingomyelin of about 5 to 40 mol percent of the total lipid present in the nanoparticle composition;
(b) a cationic lipid;
(c) a steroid;
(d) a polymer conjugated lipid; and
(e) a nucleic acid; optionally wherein the nucleic acid is mRNA.

2. The nanoparticle composition of claim 1, wherein
(a) the sphingomyelin is about 10 to 40 mol percent of the total lipid present in the nanoparticle composition,
(b) the sphingomvelin is about 10 to 30 mol percent of the total lipid present in the nanoparticle composition;
(c) the sphingomvelin is about 10 to 25 mol percent of the total lipid present in the nanoparticle composition;
(d) the sphingomvelin is about 10 to 20 mol percent of the total lipid present in the nanoparticle composition;
(e) the sphingomvelin is about 10 to 15 mol percent of the total lipid present in the nanoparticle composition;
(f) the sphingomvelin is about 10 mol percent of the total lipid present in the nanoparticle composition;
(g) the sphingomvelin is about 15 mol percent of the total lipid present in the nanoparticle composition;
or
(h) the sphingomyelin is about 20 mol percent of the total lipid present in the nanoparticle composition.

3. (canceled)

4. The nanoparticle composition of claim 1, wherein
(a) the cationic lipid is about 30 to 55 mol percent or about of the total lipid present in the nanoparticle composition;
(b) wherein the cationic lipid is about 35 to 50 mol percent of the total lipid present in the nanoparticle composition;
(c) wherein the cationic lipid is about 40 to 50 mol percent of the total lipid present in the nanoparticle composition;
(d) wherein the cationic lipid is about 45 to 50 mol percent of the total lipid present in the nanoparticle composition;
(e) wherein the cationic lipid is about 40 mol percent of the total lipid present in the nanoparticle composition;
(f) wherein the cationic lipid is about 45 mol percent of the total lipid present in the nanoparticle composition;
or
(g) wherein the cationic lipid is about 50 mol percent of the total lipid present in the nanoparticle composition.

5. (canceled)

6. The nanoparticle composition of claim 1, wherein
(a) the sphingomvelin is of about 10 to 40 mol percent of the total lipid present in the nanoparticle composition, and wherein the cationic lipid is about 30 to 55 mol percent of the total lipid present in the nanoparticle composition; or
(b) the sphingomyelin is of about 10 to 20 mol percent of the total lipid present in the nanoparticle composition, and wherein the cationic lipid is about 40 to 50 mol percent of the total lipid present in the nanoparticle composition,
(c) wherein the sphingomvelin of about 10 to 15 mol percent of the total lipid present in the nanoparticle composition, and wherein the cationic lipid is about 45 mol percent of the total lipid present in the nanoparticle composition;
(d) wherein the sphingomvelin of about 10 to 15 mol percent of the total lipid present in the nanoparticle composition, and wherein the cationic lipid is about 40 mol percent of the total lipid present in the nanoparticle composition;
(e) wherein the sphingomvelin of about 10 mol percent of the total lipid present in the nanoparticle composition, and wherein the cationic lipid is about 50 mol percent of the total lipid present in the nanoparticle composition;
(f) wherein the sphingomvelin of about 10 mol percent of the total lipid present in the nanoparticle composition, and wherein the cationic lipid is about 45 mol percent of the total lipid present in the nanoparticle composition;
or
(g) wherein the sphingomvelin of about 15 mol percent of the total lipid present in the nanoparticle composition, and wherein the cationic lipid is about 45 mol percent of the total lipid present in the nanoparticle composition.

7. (canceled)

8. The nanoparticle composition of claim 1, wherein
(a) the steroid is about 20 to 50 mol percent of the total lipid present in the nanoparticle composition;
(b) the steroid is about 30 to 50 mol percent of the total lipid present in the nanoparticle composition;
(c) the steroid is about 35 to 45 mol percent of the total lipid present in the nanoparticle composition;
(d) the steroid is about 33.5 to 43.5 mol percent of the total lipid present in the nanoparticle composition;
(e) the steroid is about 33.5 mol percent of the total lipid present in the nanoparticle composition;
(f) the steroid is about 38.5 mol percent of the total lipid present in the nanoparticle composition;
or
(g) the steroid is about 43.5 mol percent of the total lipid present in the nanoparticle composition.

9. (canceled)

10. The nanoparticle composition of claim 1, wherein
(a) the sphingomvelin is about 10 to 40 mol percent of the total lipid present in the nanoparticle composition, wherein the cationic lipid is about 30 to 55 mol percent of the total lipid present in the nanoparticle composition, and wherein the steroid is about 20 to 50 mol percent of the total lipid present in the nanoparticle composition;
(b) the sphingomyelin is about 10 to 20 mol percent of the total lipid present in the nanoparticle composition, wherein the cationic lipid is about 40 to 50 mol percent of the total lipid present in the nanoparticle composition, and wherein the steroid is about 30 to 50 mol percent of the total lipid present in the nanoparticle composition;
(c) wherein the sphingomvelin of about 10 to 20 mol percent of the total lipid present in the nanoparticle composition, wherein the cationic lipid is about 45 mol percent of the total lipid present in the nanoparticle composition; and wherein the steroid is about 33.5 to 43.5 mol percent of the total lipid present in the nanoparticle composition;
(d) wherein the sphingomvelin of about 10 to 20 mol percent of the total lipid present in the nanoparticle composition, wherein the cationic lipid is about 40 mol percent of the total lipid present in the nanoparticle composition; and wherein the steroid is about 38.5 to 48.5 mol percent of the total lipid present in the nanoparticle composition;
(e) wherein the sphingomvelin of about 10 mol percent of the total lipid present in the nanoparticle composition, wherein the cationic lipid is about 50 mol percent of the total lipid present in the nanoparticle composition, and wherein the steroid is about 38.5 mol percent of the total lipid present in the nanoparticle composition;
(f) wherein the sphingomvelin of about 10 mol percent of the total lipid present in the nanoparticle composition, wherein the cationic lipid is about 45 mol percent of the total lipid present in the nanoparticle composition; and wherein the steroid is about 43.5 mol percent of the total lipid present in the nanoparticle composition;
(g) wherein the sphingomvelin of about 15 mol percent of the total lipid present in the nanoparticle composition, wherein the cationic lipid is about 45 mol percent of the total lipid present in the nanoparticle composition, and wherein the steroid is about 38.5 mol percent of the total lipid present in the nanoparticle composition;
(h) wherein the sphingomvelin of about 20 mol percent of the total lipid present in the nanoparticle composition, wherein the cationic lipid is about 45 mol percent of the total lipid present in the nanoparticle composition, and wherein the steroid is about 33.5 mol percent of the total lipid present in the nanoparticle composition;
(i) wherein the sphingomvelin of about 10 mol percent of the total lipid present in the nanoparticle composition, wherein the cationic lipid is about 40 mol percent of the total lipid present in the nanoparticle composition, and wherein the steroid is about 48.5 mol percent of the total lipid present in the nanoparticle composition;
(i) wherein the sphingomvelin of about 15 mol percent of the total lipid present in the nanoparticle composition, wherein the cationic lipid is about 40 mol percent of the total lipid present in the nanoparticle composition, and wherein the steroid is about 43.5 mol percent of the total lipid present in the nanoparticle composition;
or
(k) wherein the sphingomvelin of about 20 mol percent of the total lipid present in the nanoparticle composition, wherein the cationic lipid is about 40 mol percent of the total lipid present in the nanoparticle composition, and wherein the steroid is about 38.5 mol percent of the total lipid present in the nanoparticle composition.

11. (canceled)

12. The nanoparticle composition of claim 1, wherein the polymer conjugated lipid is about 0.5 to 3 mol percent of the total lipid present in the nanoparticle composition or wherein the polymer conjugated lipid is about 1.5 mol percent of the total lipid present in the nanoparticle composition.

13. (canceled)

14. The nanoparticle composition of claim 1, wherein
(a) the sphingomvelin is about 10 to 40 mol percent of the total lipid present in the nanoparticle composition, wherein the cationic lipid is about 30 to 55 mol percent of the total lipid present in the nanoparticle composition, wherein the steroid is about 20 to 50 mol percent of the total lipid present in the nanoparticle composition, and wherein the polymer conjugated lipid is about 0.5 to 3 mol percent of the total lipid present in the nanoparticle composition;
(b) the sphingomyelin is about 10 to 20 mol percent of the total lipid present in the nanoparticle composition, wherein the cationic lipid is about 40 to 50 mol percent of the total lipid present in the nanoparticle composition, wherein the steroid is about 30 to 50 mol percent of the total lipid present in the nanoparticle composition, and wherein the polymer conjugated lipid is about 0.5 to 3 mol percent of the total lipid present in the nanoparticle composition;
(c) wherein the sphingomvelin is about 10 mol percent of the total lipid present in the nanoparticle composition; wherein the cationic lipid is about 50 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition;
(d) wherein the sphingomvelin is about 10 mol percent of the total lipid present in the nanoparticle composition; wherein the cationic lipid is about 45 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition;
(e) wherein the sphingomvelin is about 10 mol percent of the total lipid present in the nanoparticle composition; wherein the cationic lipid is about 40 mol percent of the total lipid present in the nanoparticle composition; wherein the steroid is about 48.5 mol percent of the total lipid present in the nanoparticle composition; and wherein the polymer conjugated lipid is about 1.5 mol percent of the total lipid present in the nanoparticle composition;
(f) wherein the sphingomvelin is about 15 mol percent of the total lipid present in the nanoparticle composition; wherein the cationic lipid is about 45 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition;
(g) wherein the sphingomyelin is about 15 mol percent of the total lipid present in the nanoparticle composition; wherein the cationic lipid is about 40 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition;
(h) wherein the sphingomyelin is about 20 mol percent of the total lipid present in the nanoparticle composition; wherein the cationic lipid is about 45 mol percent of the total lipid present in the nanoparticle composition; wherein the steroid is about 33.5 mol percent of the total lipid present in the nanoparticle composition; and wherein the polymer conjugated lipid is about 1.5 mol percent of the total lipid present in the nanoparticle composition;
(i) wherein the sphingomyelin is about 20 mol percent of the total lipid present in the nanoparticle composition; wherein the cationic lipid is about 40 mol percent of the total lipid present in the nanoparticle composition; wherein the steroid is about 38.5 mol percent of the total lipid present in the nanoparticle composition; and wherein the polymer conjugated lipid is about 1.5 mol percent of the total lipid present in the nanoparticle composition;
(j) wherein the sphingomvelin is about 5 mol percent of the total lipid present in the nanoparticle composition; wherein the cationic lipid is about 45 mol percent of the total lipid present in the nanoparticle composition; wherein the steroid is about 48.5 mol percent of the total lipid present in the nanoparticle composition; and wherein the polymer conjugated lipid is about 1.5 mol percent of the total lipid present in the nanoparticle composition;
or
(k) wherein the sphingomvelin is about 5 mol percent of the total lipid present in the nanoparticle composition; wherein the cationic lipid is about 45 mol percent of the total lipid present in the nanoparticle composition; wherein the steroid is about 43.5 mol percent of the total lipid present in the nanoparticle composition; and wherein the polymer conjugated lipid is about 1.5 mol percent of the total lipid present in the nanoparticle composition; and wherein the nanoparticle composition further comprises a second phospholipid of about 5 mol percent of the total lipid present in the nanoparticle composition; optionally wherein the second phospholipid is 1,2-distearoyl-sn-glycero-3-phosphocholine (DSPC).

15. (canceled)

16. The nanoparticle composition of claim 14, wherein
(a) the sphingomyelin is a sphingomyelin compound; optionally the sphingomyelin is selected from SM-01, SM-02, SM-03, SM-04, SM-05, SM-06 and SM-07 in Table X;
(b) the steroid is cholesterol or a cholesterol derivative;
(c) the cationic lipid is a compound according to any one of the formula selected from 01-I, 01-II, 02-I, 02-II, 03-I, 03-II-A, 03-II-B, 03-II-C, 03-II-D, 04-I, 04-III, 04-IV, 05-I, 06-I, and sub-formulas thereof, or wherein the cationic lipid is a compound selected from the compounds listed in any one of Tables 1 to 5;
and/or
(d) the polymer conjugated lipid is DMG-PEG2000 or DMPE-PEG2000.

17-19. (canceled)

20. The nanoparticle composition of claim 1, wherein the nucleic acid encodes a RNA or protein; and wherein the amount of RNA or protein expressed from the nucleic acid in a mammalian cell or tissue of a mammal is more than the amount of RNA or protein expressed from the nucleic acid formulated in a reference nanoparticle composition that does not comprise sphingomyelin of about 10 to 40 mol percent of the total lipid present in the reference nanoparticle composition; optionally wherein the reference nanoparticle composition contains 1,2-distearoyl-sn-glycero-3-phosphocholine (DSPC) instead of sphingomyelin.

21. (canceled)

22. The nanoparticle composition of claim 20, wherein the molar percentage of sphingomyelin in the total lipid present in the nanoparticle composition is the same as the molar percentage of DSPC in the total lipid present in the reference nanoparticle composition; optionally wherein remaining contents are the same between the nanoparticle composition and the reference nanoparticle composition.

23-25. (canceled)

26. The nanoparticle composition of claim 1, wherein at least about 50% the lipid nanoparticles in the plurality of lipid nanoparticles has a semi-lamellar morphology; optionally wherein at least about 55% of the lipid nanoparticles in the plurality of lipid nanoparticles have a semi-lamellar morphology.

27. The nanoparticle composition of claim 1,
(a) wherein the mean size of the plurality of lipid nanoparticles is from about 40 nm to about 150 nm; optionally wherein the mean size of the plurality of lipid nanoparticles is from about 50 nm to about 100 nm; optionally wherein the mean size of the plurality of particles is about 95 nm;
(b) wherein the encapsulation efficiency of said nucleic acid is at least about 50%; optionally wherein the encapsulation efficiency of said nucleic acid is at least about 80%; optionally wherein the encapsulation efficiency of said nucleic acid is at least about 90%;
and/or
(c) wherein the polydispersity index (PDI) of said lipid nanoparticles is from about 0 to about 0.25; optionally wherein the PDI of said lipid nanoparticles is less than 0.1; optionally wherein the PDI of said composition is less than 0.05.

28. A method of expressing an mRNA in a mammalian cell or tissue of a mammal comprising
(a) formulating the mRNA within a plurality of lipid nanoparticles in a nanoparticle composition comprising a molar ratio of about 5 to 40% sphingomyelin, about 30 to 55% cationic lipid; about 20 to 50% steroid; and about 0.5 to 3% polymer conjugated lipid;
(b) delivering the nanoparticle composition to the mammalian cells or the mammal;
and
wherein the delivered mRNA is expressed in the mammalian cell or in the mammal.

29. The method of claim 28, wherein the nanoparticle composition comprises
(a) a molar ratio of about 10 to 40% sphingomyelin, about 35 to 50% cationic lipid; about 30 to 50% steroid; and about 0.5-2 polymer conjugated lipid;
(b) a molar ratio of about 10-30% sphingomyelin, about 35 to 45% cationic lipid; about 35 to 45% steroid; and about 1.5 polymer conjugated lipid;
(c) a molar ratio of about 10% sphingomyelin, about 50% cationic lipid; about 38.5% steroid; and about 1.5% polymer conjugated lipid;
(d) a molar ratio of about 10% sphingomyelin, about 45% cationic lipid; about 43.5% steroid; and about 1.5% polymer conjugated lipid;
(e) a molar ratio of about 10% sphingomyelin, about 40% cationic lipid; about 48.5% steroid; and about 1.5% polymer conjugated lipid;
(f) a molar ratio of about 15% sphingomyelin, about 45% cationic lipid; about 38.5% steroid; and about 1.5% polymer conjugated lipid;
(g) a molar ratio of about 15% sphingomyelin, about 40% cationic lipid; about 43.5% steroid; and about 1.5% polymer conjugated lipid;
(h) a molar ratio of about 20% sphingomyelin, about 45% cationic lipid; about 33.5% steroid; and about 1.5% polymer conjugated lipid;
(i) a molar ratio of about 20% sphingomyelin, about 40% cationic lipid; about 38.5% steroid; and about 1.5% polymer conjugated lipid;
(j) a molar ratio of about 5% sphingomyelin, about 45% cationic lipid; about 48.5% steroid; and about 1.5% polymer conjugated lipid;
or
(k) a molar ratio of about 5% sphingomyelin, about 45% cationic lipid; about 43.5% steroid; about 1.5% polymer conjugated lipid, and about 5% of a second phospholipid lipid that is not sphingomyelin; wherein optionally the second phospholipid 1,2-distearoyl-sn-glycero-3-phosphocholine (DSPC).

30-33. (canceled)

34. A lipid raft nanoparticle (LRNP) comprising
(a) a sphingomyelin; and
(b) a steroid; and
at least one first lipid component that is not sphingomyelin or steroid;
wherein the LRNP has a heterogeneous structure comprising at least one liquid-ordered (Lo) domain comprising the sphingomyelin and the steroid, and at least one liquid-disordered (Ld) region comprising the first lipid component.

35. The LRNP of claim 34,
(a) wherein the Lo domain comprises a higher sphingomyelin concentration as compared to the Ld region; and/or
(b) wherein the Lo domain comprises a higher steroid concentration as compared to the Ld region.

36. The LRNP of claim 34, wherein under electron microscopy,
(a) the Ld region is electron dense;
(b) the Lo domain is not electron dense;
(c) the Lo domain assumes a uni-lamellar or multi-lamellar structure;
and/or
(d) the LRNP assumes a semi-lamellar morphorlogy.

37-40. (canceled)

41. The LRNP of claim 34, wherein
(a) the sphingomyelin is of about 5-40 mol percent of the total lipid present in the LRNP; and/or
(b) the steroid is about 20 to 50 mol percent of the total lipid present in the particle.

42. (canceled)

43. The LRNP of claim 34, wherein the first lipid component comprises
(c) a cationic lipid; optionally wherein the cationic lipid is about 30 to 55 mol percent of the total lipid present in the particle; and
(d) a polymer conjugated lipid; optionally wherein the polymer conjugated lipid is about 0.5 to 3 mol percent of the total lipid present in the particle.

44-54. (canceled)


SEQUENCE LISTING (2 sequences)

SEQ ID NO: 1 - DNA, 24 bp, synthetic construct
  source 1..24: mol_type = other DNA; note = stem-loop sequence
caaaggctcttttcagagccacca

SEQ ID NO: 2 - RNA, 24 nt, synthetic construct
  source 1..24: mol_type = other RNA; note = stem-loop sequence
caaaggctcttttcagagccacca